SEQUENCE LISTING (273 sequences)

SEQ ID NO: 1 - RNA, 25 nt, Artificial sequence
  Synthetic polynucleotide
  misc_feature (24)..(25): deoxyribose sugar
auaccuucccagguaacaaaccaac

SEQ ID NO: 2 - RNA, 25 nt, Artificial sequence
  Synthetic polynucleotide
  modified_base (2)..(2): um
  modified_base (3)..(3): 2′-O-methyladenine
  modified_base (5)..(5): cm
  modified_base (6)..(6): um
  modified_base (10)..(10): cm
  modified_base (11)..(11): 2′-O-methyladenine
  modified_base (16)..(16): 2′-O-methyladenine
  modified_base (17)..(17): cm
  misc_feature (18)..(19): phosphorothioate linkage
  misc_feature (19)..(20): phosphorothioate linkage
  misc_feature (24)..(25): deoxyribose sugar
auaccuucccagguaacaaaccaac

SEQ ID NO: 3 - RNA, 27 nt, Artificial sequence
  Synthetic polynucleotide
guugguuuguuaccugggaagguauaa

SEQ ID NO: 4 - RNA, 27 nt, Artificial sequence
  Synthetic polynucleotide
  misc_feature (1)..(2): phosphorothioate linkage
  modified_base (11)..(11): um
  modified_base (12)..(12): 2′-O-Methyladenine
  modified_base (13)..(13): cm
  modified_base (23)..(23): um
  modified_base (25)..(25): um
  modified_base (26)..(26): 2′-O-Methyladenine
guugguuuguuaccugggaagguauaa

SEQ ID NO: 5 - RNA, 25 nt, Artificial sequence
  Synthetic polynucleotide
  misc_feature (24)..(25): deoxyribose
gucccugguuucaacgagaaaacac

SEQ ID NO: 6 - RNA, 25 nt, Artificial sequence
  Synthetic polynucleotide
  modified_base (2)..(2): um
  modified_base (3)..(3): cm
  modified_base (5)..(5): cm
  modified_base (6)..(6): um
  modified_base (11)..(11): um
  modified_base (12)..(12): cm
  modified_base (14)..(14): 2′-O-Methyladenine
  modified_base (15)..(15): cm
  misc_feature (18)..(19): phosphorothioate linkage
  misc_feature (19)..(20): phosphorothioate linkage
  modified_base (23)..(23): cm
  misc_feature (24)..(25): deoxyribose sugar
gucccugguuucaacgagaaaacac

SEQ ID NO: 7 - RNA, 27 nt, Artificial sequence
  Synthetic polynucleotide
guguuuucucguugaaaccagggacaa

SEQ ID NO: 8 - RNA, 27 nt, Artificial sequence
  Synthetic polynucleotide
  modified_base (2)..(2): 2′-O-Methyladenine
  modified_base (3)..(3): cm
  modified_base (4)..(4): 2′-O-Methyladenine
  modified_base (8)..(8): 2′-O-Methyladenine
  modified_base (9)..(9): cm
  modified_base (18)..(18): cm
  modified_base (19)..(19): um
  misc_feature (26)..(27): phosphorothioate linkage
aacagggaccaaaguugcucuuuugug

SEQ ID NO: 9 - RNA, 25 nt, Artificial sequence
  Synthetic polynucleotide
cauauugcgcguauagucgcguuag

SEQ ID NO: 10 - RNA, 25 nt, Artificial sequence
  Synthetic polynucleotide
  modified_base (2)..(2): 2′-O-Methyladenine
  modified_base (3)..(3): um
  modified_base (4)..(4): 2′-O-Methyladenine
  modified_base (5)..(5): um
  modified_base (12)..(12): um
  modified_base (13)..(13): 2′-O-Methyladenine
  modified_base (14)..(14): um
  modified_base (15)..(15): 2′-O-Methyladenine
  misc_feature (18)..(19): phosphorothioate linkage
  misc_feature (19)..(20): phosphorothioate linkage
  modified_base (23)..(23): um
  misc_feature (24)..(25): deoxyribose sugar
cauauugcgcguauagucgcguuag

SEQ ID NO: 11 - RNA, 27 nt, Artificial sequence
  Synthetic polynucleotide
ugguauaacgcgcauaucagcgcaauc

SEQ ID NO: 12 - RNA, 27 nt, Artificial sequence
  Synthetic polynucleotide
  modified_base (4)..(4): um
  modified_base (6)..(6): um
  modified_base (7)..(7): 2′-O-Methyladenine
  modified_base (15)..(15): um
  modified_base (16)..(16): 2′-O-Methyladenine
  modified_base (17)..(17): um
  modified_base (18)..(18): cm
  misc_feature (26)..(27): phosphorothioate linkage
ugguauaacgcgcauaucagcgcaauc

SEQ ID NO: 13 - DNA, 25 bp, Artificial sequence
  Synthetic polynucleotide
gtccctggtttcaacgagaaaacac

SEQ ID NO: 14 - DNA, 25 bp, Artificial sequence
  Synthetic polynucleotide
gcttagtagaagttgaaaaaggcgt

SEQ ID NO: 15 - DNA, 25 bp, Artificial sequence
  Synthetic polynucleotide
tgtccctggtttcaacgagaaaaca

SEQ ID NO: 16 - DNA, 25 bp, Artificial sequence
  Synthetic polynucleotide
ataccttcccaggtaacaaaccaac

SEQ ID NO: 17 - DNA, 25 bp, Artificial sequence
  Synthetic polynucleotide
ccctggtttcaacgagaaaacacac

SEQ ID NO: 18 - DNA, 25 bp, Artificial sequence
  Synthetic polynucleotide
aaggtaagatggagagccttgtccc

SEQ ID NO: 19 - DNA, 25 bp, Artificial sequence
  Synthetic polynucleotide
aggtaagatggagagccttgtccct

SEQ ID NO: 20 - DNA, 25 bp, Artificial sequence
  Synthetic polynucleotide
ggtaagatggagagccttgtccctg

SEQ ID NO: 21 - DNA, 25 bp, Artificial sequence
  Synthetic polynucleotide
gtttacagaataaattggatcaccg

SEQ ID NO: 22 - DNA, 25 bp, Artificial sequence
  Synthetic polynucleotide
ctgtttacagaataaattggatcac

SEQ ID NO: 23 - DNA, 25 bp, Artificial sequence
  Synthetic polynucleotide
ctattaccgttgaagagcttaaaaa

SEQ ID NO: 24 - DNA, 25 bp, Artificial sequence
  Synthetic polynucleotide
gtactattaccgttgaagagcttaa

SEQ ID NO: 25 - DNA, 25 bp, Artificial sequence
  Synthetic polynucleotide
ggattggcaactataaattaaacac

SEQ ID NO: 26 - DNA, 25 bp, Artificial sequence
  Synthetic polynucleotide
ataccttcccaggtaacaaaccaac

SEQ ID NO: 27 - DNA, 28 bp, Artificial sequence
  Synthetic polynucleotide
gtccctggtttcaacgagaaaacacacg

SEQ ID NO: 28 - DNA, 40 bp, Artificial sequence
  Synthetic polynucleotide
aagatggcacttgtggcttagtagaagttgaaaaaggcgt

SEQ ID NO: 29 - DNA, 447 bp, Artificial sequence
  Synthetic polynucleotide
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgtttt

SEQ ID NO: 30 - DNA, 446 bp, Artificial sequence
  Synthetic polynucleotide
ttaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgtttt

SEQ ID NO: 31 - DNA, 446 bp, Artificial sequence; identical to SEQ ID NO: 30
  Synthetic polynucleotide

SEQ ID NO: 32 - DNA, 447 bp, Artificial sequence; identical to SEQ ID NO: 29
  Synthetic polynucleotide

SEQ ID NO: 33 - DNA, 427 bp, Artificial sequence
  Synthetic polynucleotide
caggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgtttt

SEQ ID NO: 34 - DNA, 447 bp, Artificial sequence; identical to SEQ ID NO: 29
  Synthetic polynucleotide

SEQ ID NO: 35 - DNA, 447 bp, Artificial sequence; identical to SEQ ID NO: 29
  Synthetic polynucleotide

SEQ ID NO: 36 - DNA, 447 bp, Artificial sequence; identical to SEQ ID NO: 29
  Synthetic polynucleotide

SEQ ID NO: 37 - DNA, 447 bp, Artificial sequence; identical to SEQ ID NO: 29
  Synthetic polynucleotide

SEQ ID NO: 38 - DNA, 447 bp, Artificial sequence; identical to SEQ ID NO: 29
  Synthetic polynucleotide

SEQ ID NO: 39 - DNA, 447 bp, Artificial sequence; identical to SEQ ID NO: 29
  Synthetic polynucleotide

SEQ ID NO: 40 - DNA, 393 bp, Artificial sequence
  Synthetic polynucleotide
agatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgtttt

SEQ ID NO: 41 - DNA, 300 bp, Artificial sequence
  Synthetic polynucleotide
caaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgtttt

SEQ ID NO: 42 - DNA, 446 bp, Artificial sequence
  Synthetic polynucleotide
attaaaggtttataccttcccaggtataaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgtttt

SEQ ID NO: 43 - DNA, 447 bp, Artificial sequence; identical to SEQ ID NO: 29
  Synthetic polynucleotide

SEQ ID NO: 44 - DNA, 447 bp, Artificial sequence; identical to SEQ ID NO: 29
  Synthetic polynucleotide

SEQ ID NO: 45 - DNA, 447 bp, Artificial sequence
  Synthetic polynucleotide
attaaaggtttataccttcccaggtaacaaaccaaccaactttcgatctcttgtagatctgttctctaaacgaaatttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgtttt

SEQ ID NO: 46 - DNA, 447 bp, Artificial sequence; identical to SEQ ID NO: 29
  Synthetic polynucleotide

SEQ ID NO: 47 - DNA, 447 bp, Artificial sequence; identical to SEQ ID NO: 29
  Synthetic polynucleotide

SEQ ID NO: 48 - DNA, 444 bp, Artificial sequence
  Synthetic polynucleotide
aaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgtttt

SEQ ID NO: 49 - DNA, 447 bp, Artificial sequence; identical to SEQ ID NO: 29
  Synthetic polynucleotide

SEQ ID NO: 50 - DNA, 446 bp, Artificial sequence
  Synthetic polynucleotide
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtact

SEQ ID NO: 51 - DNA, 23 bp, Artificial sequence
  Synthetic polynucleotide
ttacctacccaggaaaagccaac

SEQ ID NO: 52 - DNA, 28 bp, Artificial sequence
  Synthetic polynucleotide
tgttcttggtgtcaacgagaaaacacac

SEQ ID NO: 53 - DNA, 40 bp, Artificial sequence
  Synthetic polynucleotide
aaaatggcacttgtggtctagtagagctggaaaaaggcgt

SEQ ID NO: 54 - DNA, 30 bp, Artificial sequence
  Synthetic polynucleotide
acggtactattaccgttgaagagcttaaaa

SEQ ID NO: 55 - DNA, 27 bp, Artificial sequence
  Synthetic polynucleotide
ctgtttacagaataaattggatcaccg

SEQ ID NO: 56 - DNA, 25 bp, Artificial sequence
  Synthetic polynucleotide
ggattggcaactataaattaaacac

SEQ ID NO: 57 - DNA, 669 bp, Artificial sequence
  Synthetic polynucleotide
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 58 - DNA, 669 bp, Artificial sequence; identical to SEQ ID NO: 57
  Synthetic polynucleotide

SEQ ID NO: 59 - DNA, 669 bp, Artificial sequence; identical to SEQ ID NO: 57
  Synthetic polynucleotide

SEQ ID NO: 60 - DNA, 669 bp, Artificial sequence; identical to SEQ ID NO: 57
  Synthetic polynucleotide

SEQ ID NO: 61 - DNA, 669 bp, Artificial sequence; identical to SEQ ID NO: 57
  Synthetic polynucleotide

SEQ ID NO: 62 - DNA, 669 bp, Artificial sequence; identical to SEQ ID NO: 57
  Synthetic polynucleotide

SEQ ID NO: 63 - DNA, 669 bp, Artificial sequence; identical to SEQ ID NO: 57
  Synthetic polynucleotide

SEQ ID NO: 64 - DNA, 669 bp, Artificial sequence; identical to SEQ ID NO: 57
  Synthetic polynucleotide

SEQ ID NO: 65 - DNA, 669 bp, Artificial sequence; identical to SEQ ID NO: 57
  Synthetic polynucleotide

SEQ ID NO: 66 - DNA, 669 bp, Artificial sequence; identical to SEQ ID NO: 57
  Synthetic polynucleotide

SEQ ID NO: 67 - DNA, 669 bp, Artificial sequence; identical to SEQ ID NO: 57
  Synthetic polynucleotide

SEQ ID NO: 68 - DNA, 669 bp, Artificial sequence; identical to SEQ ID NO: 57
  Synthetic polynucleotide

SEQ ID NO: 69 - DNA, 669 bp, Artificial sequence; identical to SEQ ID NO: 57
  Synthetic polynucleotide

SEQ ID NO: 70 - DNA, 669 bp, Artificial sequence; identical to SEQ ID NO: 57
  Synthetic polynucleotide

SEQ ID NO: 71 - DNA, 669 bp, Artificial sequence; identical to SEQ ID NO: 57
  Synthetic polynucleotide

SEQ ID NO: 72 - DNA, 669 bp, Artificial sequence; identical to SEQ ID NO: 57
  Synthetic polynucleotide

SEQ ID NO: 73 - DNA, 669 bp, Artificial sequence; identical to SEQ ID NO: 57
  Synthetic polynucleotide

SEQ ID NO: 74 - DNA, 669 bp, Artificial sequence; identical to SEQ ID NO: 57
  Synthetic polynucleotide

SEQ ID NO: 75 - DNA, 669 bp, Artificial sequence; identical to SEQ ID NO: 57
  Synthetic polynucleotide

SEQ ID NO: 76 - DNA, 669 bp, Artificial sequence; identical to SEQ ID NO: 57
  Synthetic polynucleotide

SEQ ID NO: 77 - DNA, 669 bp, Artificial sequence; identical to SEQ ID NO: 57
  Synthetic polynucleotide

SEQ ID NO: 78 - DNA, 666 bp, Artificial sequence
  Synthetic polynucleotide
atggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaa

SEQ ID NO: 79 - DNA, 25 bp, Artificial sequence
  Synthetic polynucleotide
gtactattaccgttgaggagcttaa

SEQ ID NO: 80 - DNA, 28 bp, Artificial sequence
  Synthetic polynucleotide
gctgtctacagaattaattgggtgactg

SEQ ID NO: 81 - DNA, 25 bp, Artificial sequence
  Synthetic polynucleotide
gtattggaaactataaattaaatac

SEQ ID NO: 82 - DNA, 25 bp, Artificial sequence
  Synthetic polynucleotide
  misc_feature (24)..(25): deoxyribose
gcuuaguagaaguugaaaaaggcgt

SEQ ID NO: 83 - RNA, 27 nt, Artificial sequence
  Synthetic polynucleotide
acgccuuuuucaacuucuacuaagcca

SEQ ID NO: 84 - RNA, 25 nt, Artificial sequence
  Synthetic polynucleotide
  misc_feature (24)..(25): deoxyribose
aagauggcacuuguggcuuaguaga

SEQ ID NO: 85 - RNA, 27 nt, Artificial sequence
  Synthetic polynucleotide
ucuacuaagccacaagugccaucuuua

SEQ ID NO: 86 - RNA, 25 nt, Artificial sequence
  Synthetic polynucleotide
  misc_feature (24)..(25): deoxyribose
cccugguuucaacgagaaaacacac

SEQ ID NO: 87 - RNA, 27 nt, Artificial sequence
  Synthetic polynucleotide
guguguuuucucguugaaaccagggac

SEQ ID NO: 88 - RNA, 25 nt, Artificial sequence
  Synthetic polynucleotide
  misc_feature (24)..(25): deoxyribose
guuuacagaauaaauuggaucaccg

SEQ ID NO: 89 - RNA, 27 nt, Artificial sequence
  Synthetic polynucleotide
cggugauccaauuuauucuguaaacag

SEQ ID NO: 90 - RNA, 25 nt, Artificial sequence
  Synthetic polynucleotide
  misc_feature (24)..(25): deoxyribose
cuguuuacagaauaaauuggaucac

SEQ ID NO: 91 - RNA, 27 nt, Artificial sequence
  Synthetic polynucleotide
gugauccaauuuauucuguaaacagca

SEQ ID NO: 92 - RNA, 25 nt, Artificial sequence
  Synthetic polynucleotide
  misc_feature (24)..(25): deoxyribose
cuauuaccguugaagagcuuaaaaa

SEQ ID NO: 93 - RNA, 27 nt, Artificial sequence
  Synthetic polynucleotide
uuuuuaagcucuucaacgguaauagua

SEQ ID NO: 94 - RNA, 25 nt, Artificial sequence
  Synthetic polynucleotide
  misc_feature (24)..(25): deoxyribose
guacuauuaccguugaagagcuuaa

SEQ ID NO: 95 - RNA, 27 nt, Artificial sequence
  Synthetic polynucleotide
uuaagcucuucaacgguaauaguaccg

SEQ ID NO: 96 - RNA, 25 nt, Artificial sequence
  Synthetic polynucleotide
  misc_feature (24)..(25): deoxyribose
ggauuggcaacuauaaauuaaacac

SEQ ID NO: 97 - RNA, 27 nt, Artificial sequence
  Synthetic polynucleotide
guguuuaauuuauaguugccaauccug

SEQ ID NO: 98 - DNA, 474 bp, Artificial sequence
  Synthetic polynucleotide
ttaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttc

SEQ ID NO: 99 - DNA, 669 bp, Artificial sequence; identical to SEQ ID NO: 57
  Synthetic polynucleotide

SEQ ID NO: 100 - RNA, 21 nt, Artificial sequence
  Synthetic polynucleotide
cucuuguagaucuguucucua

SEQ ID NO: 101 - RNA, 21 nt, Artificial sequence
  Synthetic polynucleotide
ucucuuguagaucuguucucu

SEQ ID NO: 102 - RNA, 21 nt, Artificial sequence
  Synthetic polynucleotide
gaaagguaagauggagagccu

SEQ ID NO: 103 - RNA, 21 nt, Artificial sequence
  Synthetic polynucleotide
aaagguaagauggagagccuu

SEQ ID NO: 104 - RNA, 21 nt, Artificial sequence
  Synthetic polynucleotide
aagguaagauggagagccuug

SEQ ID NO: 105 - RNA, 21 nt, Artificial sequence
  Synthetic polynucleotide
uuaaacggguuugcgguguaa

SEQ ID NO: 106 - RNA, 21 nt, Artificial sequence
  Synthetic polynucleotide
caggauguaaacuuacauagc

SEQ ID NO: 107 - RNA, 21 nt, Artificial sequence
  Synthetic polynucleotide
guacauaaucaggauguaaac

SEQ ID NO: 108 - RNA, 21 nt, Artificial sequence
  Synthetic polynucleotide
uuacaaacaauuugauacuua

SEQ ID NO: 109 - RNA, 21 nt, Artificial sequence
  Synthetic polynucleotide
uuuacaaacaauuugauacuu

SEQ ID NO: 110 - RNA, 21 nt, Artificial sequence
  Synthetic polynucleotide
auggagucacauuaauuggag

SEQ ID NO: 111 - RNA, 21 nt, Artificial sequence
  Synthetic polynucleotide
uggagucacauuaauuggaga

SEQ ID NO: 112 - RNA, 21 nt, Artificial sequence
  Synthetic polynucleotide
ugguuuuaauuuuucacaaau

SEQ ID NO: 113 - RNA, 21 nt, Artificial sequence
  Synthetic polynucleotide
gugguuuuaauuuuucacaaa

SEQ ID NO: 114 - RNA, 21 nt, Artificial sequence
  Synthetic polynucleotide
gcuuguuuugugcuugcugcu

SEQ ID NO: 115 - RNA, 21 nt, Artificial sequence
  Synthetic polynucleotide
uuguuuugugcuugcugcugu

SEQ ID NO: 116 - RNA, 21 nt, Artificial sequence
  Synthetic polynucleotide
aguaauagguuuccuauuccu

SEQ ID NO: 117 - RNA, 21 nt, Artificial sequence
  Synthetic polynucleotide
guaauagguuuccuauuccuu

SEQ ID NO: 118 - RNA, 21 nt, Artificial sequence
  Synthetic polynucleotide
aacgaacaugaaaauuauucu

SEQ ID NO: 119 - RNA, 21 nt, Artificial sequence
  Synthetic polynucleotide
aaggccaaacugucacuaaga

SEQ ID NO: 120 - RNA, 21 nt, Artificial sequence
  Synthetic polynucleotide
gccaaacugucacuaagaaau

SEQ ID NO: 121 - RNA, 21 nt, Artificial sequence
  Synthetic polynucleotide
cuuauggguugggauuauccu

SEQ ID NO: 122 - RNA, 21 nt, Artificial sequence
  Synthetic polynucleotide
ugcaaagaauagagcucgcac

SEQ ID NO: 123 - RNA, 21 nt, Artificial sequence
  Synthetic polynucleotide
acuguugauucaucacagggc

SEQ ID NO: 124 - RNA, 21 nt, Artificial sequence
  Synthetic polynucleotide
ucaccuuauaauucacagaau

SEQ ID NO: 125 - RNA, 21 nt, Artificial sequence
  Synthetic polynucleotide
cuuguagaucuguucucuaaa

SEQ ID NO: 126 - RNA, 21 nt, Artificial sequence
  Synthetic polynucleotide
uagagaacagaucuacaagag

SEQ ID NO: 127 - RNA, 21 nt, Artificial sequence
  Synthetic polynucleotide
agagaacagaucuacaagaga

SEQ ID NO: 128 - RNA, 21 nt, Artificial sequence
  Synthetic polynucleotide
aggcucuccaucuuaccuuuc

SEQ ID NO: 129 - RNA, 21 nt, Artificial sequence
  Synthetic polynucleotide
agguaagauggagagccuugu

SEQ ID NO: 130 - RNA, 21 nt, Artificial sequence
  Synthetic polynucleotide
aaggcucuccaucuuaccuuu

SEQ ID NO: 131 - RNA, 21 nt, Artificial sequence
  Synthetic polynucleotide
gguaagauggagagccuuguc

SEQ ID NO: 132 - RNA, 21 nt, Artificial sequence
  Synthetic polynucleotide
caaggcucuccaucuuaccuu

SEQ ID NO: 133 - DNA, 21 bp, Artificial sequence
  Synthetic polynucleotide
aaacgggtttgcggtgtaagt

SEQ ID NO: 134 - RNA, 21 nt, Artificial sequence
  Synthetic polynucleotide
uuacaccgcaaacccguuuaa

SEQ ID NO: 135 - RNA, 21 nt, Artificial sequence
  Synthetic polynucleotide
ggauguaaacuuacauagcuc

SEQ ID NO: 136 - RNA, 21 nt, Artificial sequence
  Synthetic polynucleotide
gcuauguaaguuuacauccug

SEQ ID NO: 137 - RNA, 21 nt, Artificial sequence
  Synthetic polynucleotide
acauaaucaggauguaaacuu

SEQ ID NO: 138 - RNA, 21 nt, Artificial sequence
  Synthetic polynucleotide
guuuacauccugauuauguac

SEQ ID NO: 139 - RNA, 21 nt, Artificial sequence
  Synthetic polynucleotide
acaaacaauuugauacuuaua

SEQ ID NO: 140 - RNA, 21 nt, Artificial sequence
  Synthetic polynucleotide
uaaguaucaaauuguuuguaa

SEQ ID NO: 141 - RNA, 21 nt, Artificial sequence
  Synthetic polynucleotide
uacaaacaauuugauacuuau

SEQ ID NO: 142 - RNA, 21 nt, Artificial sequence
  Synthetic polynucleotide
aaguaucaaauuguuuguaaa

SEQ ID NO: 143 - RNA, 21 nt, Artificial sequence
  Synthetic polynucleotide
ggagucacauuaauuggagaa

SEQ ID NO: 144 - RNA, 21 nt, Artificial sequence
  Synthetic polynucleotide
cuccaauuaaugugacuccau

SEQ ID NO: 145 - RNA, 21 nt, Artificial sequence
  Synthetic polynucleotide
gagucacauuaauuggagaag

SEQ ID NO: 146 - RNA, 21 nt, Artificial sequence
  Synthetic polynucleotide
ucuccaauuaaugugacucca

SEQ ID NO: 147 - RNA, 21 nt, Artificial sequence
  Synthetic polynucleotide
guuuuaauuuuucacaaauau

SEQ ID NO: 148 - RNA, 21 nt, Artificial sequence
  Synthetic polynucleotide
auuugugaaaaauuaaaacca

SEQ ID NO: 149 - RNA, 21 nt, Artificial sequence
  Synthetic polynucleotide
gguuuuaauuuuucacaaaua

SEQ ID NO: 150 - RNA, 21 nt, Artificial sequence
  Synthetic polynucleotide
uuugugaaaaauuaaaaccac

SEQ ID NO: 151 - RNA, 21 nt, Artificial sequence
  Synthetic polynucleotide
agcagcaagcacaaaacaagc

SEQ ID NO: 152 - RNA, 21 nt, Artificial sequence
  Synthetic polynucleotide
guuuugugcuugcugcuguuu

SEQ ID NO: 153 - RNA, 21 nt, Artificial sequence
  Synthetic polynucleotide
acagcagcaagcacaaaacaa

SEQ ID NO: 154 - RNA, 21 nt, Artificial sequence
  Synthetic polynucleotide
uaauagguuuccuauuccuua

SEQ ID NO: 155 - RNA, 21 nt, Artificial sequence
  Synthetic polynucleotide
aggaauaggaaaccuauuacu

SEQ ID NO: 156 - RNA, 21 nt, Artificial sequence
  Synthetic polynucleotide
aauagguuuccuauuccuuac

SEQ ID NO: 157 - RNA, 21 nt, Artificial sequence
  Synthetic polynucleotide
aaggaauaggaaaccuauuac

SEQ ID NO: 158 - RNA, 21 nt, Artificial sequence
  Synthetic polynucleotide
cgaacaugaaaauuauucuuu

SEQ ID NO: 159 - RNA, 21 nt, Artificial sequence
  Synthetic polynucleotide
agaauaauuuucauguucguu

SEQ ID NO: 160 - RNA, 21 nt, Artificial sequence
  Synthetic polynucleotide
ggccaaacugucacuaagaaa

SEQ ID NO: 161 - RNA, 21 nt, Artificial sequence
  Synthetic polynucleotide
ucuuagugacaguuuggccuu

SEQ ID NO: 162 - RNA, 21 nt, Artificial sequence
  Synthetic polynucleotide
caaacugucacuaagaaaucu

SEQ ID NO: 163 - RNA, 21 nt, Artificial sequence
  Synthetic polynucleotide
auuucuuagugacaguuuggc

SEQ ID NO: 164 - RNA, 25 nt, Artificial sequence
  Synthetic polynucleotide
  misc_feature (24)..(25): deoxyribose
cuuguagaucuguucucuaaacgaa

SEQ ID NO: 165 - RNA, 27 nt, Artificial sequence
  Synthetic polynucleotide
uucguuuagagaacagaucuacaagag

SEQ ID NO: 166 - RNA, 25 nt, Artificial sequence
  Synthetic polynucleotide
  misc_feature (24)..(25): deoxyribose
ucuuguagaucuguucucuaaacga

SEQ ID NO: 167 - RNA, 27 nt, Artificial sequence
  Synthetic polynucleotide
ucguuuagagaacagaucuacaagaga

SEQ ID NO: 168 - RNA, 25 nt, Artificial sequence
  Synthetic polynucleotide
  misc_feature (24)..(25): deoxyribose
aagguaagauggagagccuuguccc

SEQ ID NO: 169 - RNA, 27 nt, Artificial sequence
  Synthetic polynucleotide
gggacaaggcucuccaucuuaccuuuc

SEQ ID NO: 170 - DNA, 25 bp, Artificial sequence
  Synthetic polynucleotide
  misc_feature (24)..(25): deoxyribose
agguaagauggagagccuuguccct

SEQ ID NO: 171 - RNA, 27 nt, Artificial sequence
  Synthetic polynucleotide
agggacaaggcucuccaucuuaccuuu

SEQ ID NO: 172 - DNA, 25 bp, Artificial sequence
  Synthetic polynucleotide
  misc_feature (24)..(25): deoxyribose
gguaagauggagagccuuguccctg

SEQ ID NO: 173 - RNA, 27 nt, Artificial sequence
  Synthetic polynucleotide
cagggacaaggcucuccaucuuaccuu

SEQ ID NO: 174 - DNA, 25 bp, Artificial sequence
  Synthetic polynucleotide
  misc_feature (24)..(25): deoxyribose
aaacgggtttgcggtgtaagtgcag

SEQ ID NO: 175 - RNA, 27 nt, Artificial sequence
  Synthetic polynucleotide
cugcacuuacaccgcaaacccguuuaa

SEQ ID NO: 176 - RNA, 25 nt, Artificial sequence
  Synthetic polynucleotide
  misc_feature (24)..(25): deoxyribose
ggauguaaacuuacauagcucuaga

SEQ ID NO: 177 - RNA, 27 nt, Artificial sequence
  Synthetic polynucleotide
ucuagagcuauguaaguuuacauccug

SEQ ID NO: 178 - DNA, 25 bp, Artificial sequence
  Synthetic polynucleotide
  misc_feature (24)..(25): deoxyribose
acauaaucaggauguaaacuuacat

SEQ ID NO: 179 - RNA, 27 nt, Artificial sequence
  Synthetic polynucleotide
auguaaguuuacauccugauuauguac

SEQ ID NO: 180 - DNA, 25 bp, Artificial sequence
  Synthetic polynucleotide
  misc_feature (24)..(25): deoxyribose
acaaacaauuugauacuuauaacct

SEQ ID NO: 181 - RNA, 27 nt, Artificial sequence
  Synthetic polynucleotide
agguuauaaguaucaaauuguuuguaa

SEQ ID NO: 182 - RNA, 25 nt, Artificial sequence
  Synthetic polynucleotide
  misc_feature (24)..(25): deoxyribose
uacaaacaauuugauacuuauaacc

SEQ ID NO: 183 - RNA, 27 nt, Artificial sequence
  Synthetic polynucleotide
gguuauaaguaucaaauuguuuguaaa

SEQ ID NO: 184 - RNA, 25 nt, Artificial sequence
  Synthetic polynucleotide
  misc_feature (24)..(25): deoxyribose
ggagucacauuaauuggagaagccg

SEQ ID NO: 185 - RNA, 27 nt, Artificial sequence
  Synthetic polynucleotide
cggcuucuccaauuaaugugacuccau

SEQ ID NO: 186 - DNA, 25 bp, Artificial sequence
  Synthetic polynucleotide
  misc_feature (24)..(25): deoxyribose
gagucacauuaauuggagaagccgt

SEQ ID NO: 187 - RNA, 27 nt, Artificial sequence
  Synthetic polynucleotide
acggcuucuccaauuaaugugacucca

SEQ ID NO: 188 - RNA, 25 nt, Artificial sequence
  Synthetic polynucleotide
  misc_feature (24)..(25): deoxyribose
guuuuaauuuuucacaaauauuacc

SEQ ID NO: 189 - RNA, 27 nt, Artificial sequence
  Synthetic polynucleotide
gguaauauuugugaaaaauuaaaacca

SEQ ID NO: 190 - RNA, 25 nt, Artificial sequence
  Synthetic polynucleotide
  misc_feature (24)..(25): deoxyribose
gguuuuaauuuuucacaaauauuac

SEQ ID NO: 191 - RNA, 27 nt, Artificial sequence
  Synthetic polynucleotide
guaauauuugugaaaaauuaaaaccac

SEQ ID NO: 192 - RNA, 25 nt, Artificial sequence
  Synthetic polynucleotide
  misc_feature (24)..(25): deoxyribose
uuguuuugugcuugcugcuguuuac

SEQ ID NO: 193 - RNA, 27 nt, Artificial sequence
  Synthetic polynucleotide
guaaacagcagcaagcacaaaacaagc

SEQ ID NO: 194 - RNA, 25 nt, Artificial sequence
  Synthetic polynucleotide
  misc_feature (24)..(25): deoxyribose
guuuugugcuugcugcuguuuacag

SEQ ID NO: 195 - RNA, 27 nt, Artificial sequence
  Synthetic polynucleotide
cuguaaacagcagcaagcacaaaacaa

SEQ ID NO: 196 - DNA, 25 bp, Artificial sequence
  Synthetic polynucleotide
  misc_feature (24)..(25): deoxyribose
uaauagguuuccuauuccuuacatg

SEQ ID NO: 197 - RNA, 27 nt, Artificial sequence
  Synthetic polynucleotide
cauguaaggaauaggaaaccuauuacu

SEQ ID NO: 198 - RNA, 25 nt, Artificial sequence
  Synthetic polynucleotide
  misc_feature (24)..(25): deoxyribose
aauagguuuccuauuccuuacaugg

SEQ ID NO: 199 - RNA, 27 nt, Artificial sequence
  Synthetic polynucleotide
ccauguaaggaauaggaaaccuauuac

SEQ ID NO: 200 - DNA, 25 bp, Artificial sequence
  Synthetic polynucleotide
  misc_feature (24)..(25): deoxyribose
cgaacaugaaaauuauucuuuuctt

SEQ ID NO: 201 - RNA, 27 nt, Artificial sequence
  Synthetic polynucleotide
aagaaaagaauaauuuucauguucguu

SEQ ID NO: 202 - DNA, 25 bp, Artificial sequence
  Synthetic polynucleotide
  misc_feature (24)..(25): deoxyribose
ggccaaacugucacuaagaaauctg

SEQ ID NO: 203 - RNA, 27 nt, Artificial sequence
  Synthetic polynucleotide
cagauuucuuagugacaguuuggccuu

SEQ ID NO: 204 - DNA, 25 bp, Artificial sequence
  Synthetic polynucleotide
  misc_feature (24)..(25): deoxyribose
caaacugucacuaagaaaucugctg

SEQ ID NO: 205 - RNA, 27 nt, Artificial sequence
  Synthetic polynucleotide
cagcagauuucuuagugacaguuuggc

SEQ ID NO: 206 - RNA, 21 nt, Artificial sequence
  Synthetic polynucleotide
uauggguugggauuauccuaa

SEQ ID NO: 207 - RNA, 21 nt, Artificial sequence
  Synthetic polynucleotide
aggauaaucccaacccauaag

SEQ ID NO: 208 - RNA, 21 nt, Artificial sequence
  Synthetic polynucleotide
caaagaauagagcucgcaccg

SEQ ID NO: 209 - RNA, 21 nt, Artificial sequence
  Synthetic polynucleotide
gugcgagcucuauucuuugca

SEQ ID NO: 210 - RNA, 21 nt, Artificial sequence
  Synthetic polynucleotide
uguugauucaucacagggcuc

SEQ ID NO: 211 - RNA, 21 nt, Artificial sequence
  Synthetic polynucleotide
gcccugugaugaaucaacagu

SEQ ID NO: 212 - RNA, 21 nt, Artificial sequence
  Synthetic polynucleotide
accuuauaauucacagaaugc

SEQ ID NO: 213 - RNA, 21 nt, Artificial sequence
  Synthetic polynucleotide
auucugugaauuauaagguga

SEQ ID NO: 214 - DNA, 25 bp, Artificial sequence
  Synthetic polynucleotide
  misc_feature (24)..(25): deoxyribose
uauggguugggauuauccuaaaugt

SEQ ID NO: 215 - RNA, 27 nt, Artificial sequence
  Synthetic polynucleotide
acauuuaggauaaucccaacccauaag

SEQ ID NO: 216 - RNA, 25 nt, Artificial sequence
  Synthetic polynucleotide
  misc_feature (24)..(25): deoxyribose
caaagaauagagcucgcaccguagc

SEQ ID NO: 217 - RNA, 27 nt, Artificial sequence
  Synthetic polynucleotide
gcuacggugcgagcucuauucuuugca

SEQ ID NO: 218 - RNA, 25 nt, Artificial sequence
  Synthetic polynucleotide
  misc_feature (24)..(25): deoxyribose
uguugauucaucacagggcucagaa

SEQ ID NO: 219 - RNA, 27 nt, Artificial sequence
  Synthetic polynucleotide
uucugagcccugugaugaaucaacagu

SEQ ID NO: 220 - RNA, 25 nt, Artificial sequence
  Synthetic polynucleotide
  misc_feature (24)..(25): deoxyribose
accuuauaauucacagaaugcugua

SEQ ID NO: 221 - RNA, 27 nt, Artificial sequence
  Synthetic polynucleotide
uacagcauucugugaauuauaagguga

SEQ ID NO: 222 - DNA, 400 bp, Artificial sequence
  Synthetic polynucleotide
tgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgct

SEQ ID NO: 223 - RNA, 21 nt, Artificial sequence
  Synthetic polynucleotide
uugucccugguuucaacgaga

SEQ ID NO: 224 - RNA, 21 nt, Artificial sequence
  Synthetic polynucleotide
uuauaccuucccagguaacaa

SEQ ID NO: 225 - RNA, 21 nt, Artificial sequence
  Synthetic polynucleotide
cgaacuuauguacucauucgu

SEQ ID NO: 226 - RNA, 21 nt, Artificial sequence
  Synthetic polynucleotide
gaacuuauguacucauucguu

SEQ ID NO: 227 - RNA, 21 nt, Artificial sequence
  Synthetic polynucleotide
ucuuugaauguggcuaaaucu

SEQ ID NO: 228 - RNA, 21 nt, Artificial sequence
  Synthetic polynucleotide
gaaccaccuuguagguuuguu

SEQ ID NO: 229 - RNA, 21 nt, Artificial sequence
  Synthetic polynucleotide
acuggaaccaccuuguagguu

SEQ ID NO: 230 - RNA, 21 nt, Artificial sequence
  Synthetic polynucleotide
ggaaccaccuuguagguuugu

SEQ ID NO: 231 - RNA, 21 nt, Artificial sequence
  Synthetic polynucleotide
guuggacugagacugaccuua

SEQ ID NO: 232 - RNA, 21 nt, Artificial sequence
  Synthetic polynucleotide
gaacuggaaccaccuuguagg

SEQ ID NO: 233 - RNA, 21 nt, Artificial sequence
  Synthetic polynucleotide
ugaugaugauuauuucaauaa

SEQ ID NO: 234 - RNA, 21 nt, Artificial sequence
  Synthetic polynucleotide
ucacuauauguuaaaccaggu

SEQ ID NO: 235 - RNA, 21 nt, Artificial sequence
  Synthetic polynucleotide
cuuauggguugggauuacccu

SEQ ID NO: 236 - DNA, 25 bp, Artificial sequence
  Synthetic polynucleotide
  misc_feature (24)..(25): deoxyribose
uauggguugggauuacccuaaaugt

SEQ ID NO: 237 - RNA, 27 nt, Artificial sequence
  Synthetic polynucleotide
acauuuaggguaaucccaacccauaag

SEQ ID NO: 238 - RNA, 21 nt, Artificial sequence
  Synthetic polynucleotide
aacuuauguacucauucguuu

SEQ ID NO: 239 - RNA, 21 nt, Artificial sequence
  Synthetic polynucleotide
acgaaugaguacauaaguucg

SEQ ID NO: 240 - RNA, 21 nt, Artificial sequence
  Synthetic polynucleotide
acuuauguacucauucguuuc

SEQ ID NO: 241 - RNA, 21 nt, Artificial sequence
  Synthetic polynucleotide
aacgaaugaguacauaaguuc

SEQ ID NO: 242 - RNA, 21 nt, Artificial sequence
  Synthetic polynucleotide
uuugaauguggcuaaaucuga

SEQ ID NO: 243 - RNA, 21 nt, Artificial sequence
  Synthetic polynucleotide
agauuuagccacauucaaaga

SEQ ID NO: 244 - RNA, 21 nt, Artificial sequence
  Synthetic polynucleotide
accaccuuguagguuuguuac

SEQ ID NO: 245 - RNA, 21 nt, Artificial sequence
  Synthetic polynucleotide
aacaaaccuacaaggugguuc

SEQ ID NO: 246 - RNA, 21 nt, Artificial sequence
  Synthetic polynucleotide
uggaaccaccuuguagguuug

SEQ ID NO: 247 - RNA, 21 nt, Artificial sequence
  Synthetic polynucleotide
aaccuacaaggugguuccagu

SEQ ID NO: 248 - RNA, 21 nt, Artificial sequence
  Synthetic polynucleotide
aaccaccuuguagguuuguua

SEQ ID NO: 249 - RNA, 21 nt, Artificial sequence
  Synthetic polynucleotide
acaaaccuacaaggugguucc

SEQ ID NO: 250 - RNA, 21 nt, Artificial sequence
  Synthetic polynucleotide
uggacugagacugaccuuacu

SEQ ID NO: 251 - RNA, 21 nt, Artificial sequence
  Synthetic polynucleotide
uaaggucagucucaguccaac

SEQ ID NO: 252 - RNA, 21 nt, Artificial sequence
  Synthetic polynucleotide
acuggaaccaccuuguagguu

SEQ ID NO: 253 - RNA, 21 nt, Artificial sequence
  Synthetic polynucleotide
ccuacaaggugguuccaguuc

SEQ ID NO: 254 - RNA, 21 nt, Artificial sequence
  Synthetic polynucleotide
augaugauuauuucaauaaaa

SEQ ID NO: 255 - RNA, 21 nt, Artificial sequence
  Synthetic polynucleotide
uuauugaaauaaucaucauca

SEQ ID NO: 256 - RNA, 21 nt, Artificial sequence
  Synthetic polynucleotide
acuauauguuaaaccaggugg

SEQ ID NO: 257 - RNA, 21 nt, Artificial sequence
  Synthetic polynucleotide
accugguuuaacauauaguga

SEQ ID NO: 258 - RNA, 21 nt, Artificial sequence
  Synthetic polynucleotide
uggcuuaguagaaguugaaaa

SEQ ID NO: 259 - RNA, 21 nt, Artificial sequence
  Synthetic polynucleotide
uaaagauggcacuuguggcuu

SEQ ID NO: 260 - RNA, 21 nt, Artificial sequence
  Synthetic polynucleotide
gucccugguuucaacgagaaa

SEQ ID NO: 261 - RNA, 21 nt, Artificial sequence
  Synthetic polynucleotide
cuguuuacagaauaaauugga

SEQ ID NO: 262 - RNA, 21 nt, Artificial sequence
  Synthetic polynucleotide
ugcuguuuacagaauaaauug

SEQ ID NO: 263 - RNA, 21 nt, Artificial sequence
  Synthetic polynucleotide
uacuauuaccguugaagagcu

SEQ ID NO: 264 - RNA, 21 nt, Artificial sequence
  Synthetic polynucleotide
cgguacuauuaccguugaaga

SEQ ID NO: 265 - RNA, 21 nt, Artificial sequence
  Synthetic polynucleotide
caggauuggcaacuauaaauu

SEQ ID NO: 266 - DNA, 600 bp, Artificial sequence
  Synthetic polynucleotide
cttgtctgattagttcctggtccccaaaatttccttgggtttgttctggaccacgtctgccgaaagcttgtgttacattgtatgctttagtggcagtacgtttttgccgaggcttcttagaagcctcagcagcagatttcttagtgacagtttggccttgttgttgttggcctttaccagacattttgctctcaagctggttcaatctgtcaagcagcagcaaagcaagagcagcatcaccgccattgccagccattctagcaggagaagttcccctactgctgcctggagttgaatttcttgaactgttgcgactacgtgatgaggaacgagaagaggcttgactgccgcctctgctcccttctgcgtagaagccttttggcaatgttgttccttgaggaagttgtagcacgattgcagcattgttagcaggattgcgggtgccaatgtgatcttttggtgtattcaaggctccctcagttgcaacccatatgatgccgtctttgttagcaccatagggaagtccagcttctggcccagttcctaggtagtagaaataccatcttggactgagatctttcattttaccgtcaccaccac

SEQ ID NO: 267 - DNA, 800 bp, Artificial sequence
  Synthetic polynucleotide
tttgtatgcgtcaatatgcttattcagcaaaatgacttgatctttgaaatttggatctttgtcatccaatttgatggcacctgtgtaggtcaaccacgttcccgaaggtgtgacttccatgccaatgcgcgacattccgaagaacgctgaagcgctgggggcaaattgtgcaatttgcggccaatgtttgtaatcagttccttgtctgattagttcctggtccccaaaatttccttgggtttgttctggaccacgtctgccgaaagcttgtgttacattgtatgctttagtggcagtacgtttttgccgaggcttcttagaagcctcagcagcagatttcttagtgacagtttggccttgttgttgttggcctttaccagacattttgctctcaagctggttcaatctgtcaagcagcagcaaagcaagagcagcatcaccgccattgccagccattctagcaggagaagttcccctactgctgcctggagttgaatttcttgaactgttgcgactacgtgatgaggaacgagaagaggcttgactgccgcctctgctcccttctgcgtagaagccttttggcaatgttgttccttgaggaagttgtagcacgattgcagcattgttagcaggattgcgggtgccaatgtgatcttttggtgtattcaaggctccctcagttgcaacccatatgatgccgtctttgttagcaccatagggaagtccagcttctggcccagttcctaggtagtagaaataccatcttggactgagatctttcattttaccgtcaccaccac

SEQ ID NO: 268 - DNA, 400 bp, Artificial sequence
  Synthetic polynucleotide
tctgccattgtgtatttagtaagacgttgacgtgatatatgtggtaccatgtcaccgtctattctaaacttaaagaagtcatgtttagcaacagctggacaatccttaagtaaattataaattgtttcttcatgttggtagttagagaaagtgtgtctcttaactacaaagtaagaatcaattaaattgtcatcttcgtccttttcttggaagcgacaacaattagtttttaggaatttagcaaaaccagctactttatcattgtagatgtcaaaagccctgtatacgacatcagtactagtgcctgtgccgcacggtgtaagacgggctgcacttacaccgcaaacccgtttaaaaacgattgtgcatcagctgactgaagcatgggttcgcggagttg

SEQ ID NO: 269 - DNA, 600 bp, Artificial sequence
  Synthetic polynucleotide
agaattgtactgtttttaacaaagcttggcgtacacgttcacctaagttggcgtatacgcgtaatatatctgggttttctacaaaatcataccagtcctttttattgaaataatcatcatcacaacaattgtatgtgacaagtatttcttttaatgtgtcacaattaccttcatcaaaatgccttaaagcatagacgaggtctgccattgtgtatttagtaagacgttgacgtgatatatgtggtaccatgtcaccgtctattctaaacttaaagaagtcatgtttagcaacagctggacaatccttaagtaaattataaattgtttcttcatgttggtagttagagaaagtgtgtctcttaactacaaagtaagaatcaattaaattgtcatcttcgtccttttcttggaagcgacaacaattagtttttaggaatttagcaaaaccagctactttatcattgtagatgtcaaaagccctgtatacgacatcagtactagtgcctgtgccgcacggtgtaagacgggctgcacttacaccgcaaacccgtttaaaaacgattgtgcatcagctgactgaagcatgggttcgcggagttg

SEQ ID NO: 270 - DNA, 800 bp, Artificial sequence
  Synthetic polynucleotide
agtgtcaacatgtgactctgcagttaaagccctggtcaaggttaatataggcattaacaatgaataataagaatctacaacaggaactccactacctggcgtggtttgtatgaaatcaccgaaatcataccagttaccattgagatcttgattatctaatgtcagtacaccaacaataccagcatttcgcatggcatcacagaattgtactgtttttaacaaagcttggcgtacacgttcacctaagttggcgtatacgcgtaatatatctgggttttctacaaaatcataccagtcctttttattgaaataatcatcatcacaacaattgtatgtgacaagtatttcttttaatgtgtcacaattaccttcatcaaaatgccttaaagcatagacgaggtctgccattgtgtatttagtaagacgttgacgtgatatatgtggtaccatgtcaccgtctattctaaacttaaagaagtcatgtttagcaacagctggacaatccttaagtaaattataaattgtttcttcatgttggtagttagagaaagtgtgtctcttaactacaaagtaagaatcaattaaattgtcatcttcgtccttttcttggaagcgacaacaattagtttttaggaatttagcaaaaccagctactttatcattgtagatgtcaaaagccctgtatacgacatcagtactagtgcctgtgccgcacggtgtaagacgggctgcacttacaccgcaaacccgtttaaaaacgattgtgcatcagctgactgaagcatgggttcgcggagttg

SEQ ID NO: 271 - DNA, 400 bp, Artificial sequence
  Synthetic polynucleotide
taaagaactgacttaaagttctttatgctagccactagaccttgagatgcataagtgctattgaaacacacaacagcatcgtcagagagtatcatcattgagaaatgtttacgcaaatatgcgtaaaactcattcacaaagtctgtgtcaacatctctatttctatagagacactcataaagtctgtgttgtaaattgcggacatacttatcggcaattttgttaccatcagtagataaaagtgcattaacattggccgtgacagcttgacaaatgttaaaaacactattagcataagcagttgtggcatctcctgatgaggttccacctggtttaacatatagtgaaccgccacacatgaccatttcactcaatacttgagcacactcattagctaatc

SEQ ID NO: 272 - DNA, 600 bp, Artificial sequence
  Synthetic polynucleotide
taaagaactgacttaaagttctttatgctagccactagaccttgagatgcataagtgctattgaaacacacaacagcatcgtcagagagtatcatcattgagaaatgtttacgcaaatatgcgtaaaactcattcacaaagtctgtgtcaacatctctatttctatagagacactcataaagtctgtgttgtaaattgcggacatacttatcggcaattttgttaccatcagtagataaaagtgcattaacattggccgtgacagcttgacaaatgttaaaaacactattagcataagcagttgtggcatctcctgatgaggttccacctggtttaacatatagtgaaccgccacacatgaccatttcactcaatacttgagcacactcattagctaatctatagaaacggtgtgacaagctacaacacgttgtatgtttgcgagcaagaacaagtgaggccataattctaagcatgttaggcatggctctatcacatttaggataatcccaacccataaggtgagggttttctacatcactataaacagtttttaacatgttgtgccaaccaccatagaatttgcttgttccaattact

SEQ ID NO: 273 - DNA, 800 bp, Artificial sequence
  Synthetic polynucleotide
taaagaactgacttaaagttctttatgctagccactagaccttgagatgcataagtgctattgaaacacacaacagcatcgtcagagagtatcatcattgagaaatgtttacgcaaatatgcgtaaaactcattcacaaagtctgtgtcaacatctctatttctatagagacactcataaagtctgtgttgtaaattgcggacatacttatcggcaattttgttaccatcagtagataaaagtgcattaacattggccgtgacagcttgacaaatgttaaaaacactattagcataagcagttgtggcatctcctgatgaggttccacctggtttaacatatagtgaaccgccacacatgaccatttcactcaatacttgagcacactcattagctaatctatagaaacggtgtgacaagctacaacacgttgtatgtttgcgagcaagaacaagtgaggccataattctaagcatgttaggcatggctctatcacatttaggataatcccaacccataaggtgagggttttctacatcactataaacagtttttaacatgttgtgccaaccaccatagaatttgcttgttccaattactacagtagctcctctagtggcggctattgatttcaataatttttgatgaaactgtctattggtcatagtactacagatagagacaccagctacggtgcgagctctattctttgcactaatggcatacttaagattcatttgagttatagtagggatgacattacgttttgtatatgcgaaaagtgcatcttgatcctcata

(19) United States
(12) Patent Application Publication
(10) Pub. No.: US 2023/0323353 A1
(43) Pub. Date: Oct. 12, 2023

(54) ANTISENSE RNA FOR TREATMENT OF SARS-ASSOCIATED CORONAVIRUS

(71) Applicant: City of Hope, Duarte, CA (US)

(72) Inventors: Kevin Morris, Duarte, CA (US); Tristan Scott, Duarte, CA (US); Roslyn Ray, Duarte, CA (US); Galina Shevchenko, Duarte, CA (US); Denis O'Meally, Duarte, CA (US)

(21) Appl. No.: 17/912,447

(22) PCT Filed: Mar. 23, 2021
(86) PCT No.: PCT/US2021/023593

§ 371 Date: Sep. 16, 2022

Related U.S. Application Data
(60) Provisional application No. 63/005,856, filed on Apr. 6, 2020.
(60) Provisional application No. 62/994,774, filed on Mar. 25, 2020.

(51) Int. Cl. C12N 15/113; A61K 9/51; A61P 31/14

(52) U.S. Cl. CPC C12N 15/1131; A61K 9/5123; A61P 31/14; C12N 2310/11; C12N 2310/3515; C12N 2310/14

(57) ABSTRACT

Provided herein are, inter alia, nucleic acids (e.g., siRNA), lipid nanoparticles comprising nucleic acids, pharmaceutical compositions comprising nucleic acids, pharmaceutical compositions comprising lipid nanoparticles, and methods of treating COV-ID-19, SARS viruses, and Middle Eastern respiratory syndrome.

### CROSS-REFERENCE TO RELATED APPLICATIONS

This application claims priority to U.S. Application No. 63/005,856 filed Apr. 6, 2020, and U.S. Application No. 62/994,774 filed Mar. 25, 2020, the disclosures of which are incorporated by reference herein in their entirety.

### STATEMENT AS TO RIGHTS TO INVENTIONS MADE UNDER FEDERALLY SPONSORED RESEARCH AND DEVELOPMENT

This invention was made with government support under grant no. MH113407 awarded by National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048440-762001WO_SequenceListing_ST25.txt, created Mar. 12, 2021, 99,602 bytes, machine format IBM-PC, MS Windows operating system, is hereby incorporated by reference.

### BACKGROUND

SARS-CoV-2 has emerged as a highly infectious agent that can rapidly spread geographically and has a mortality rate of about 2-3.0%. See Ji et al, Lancet Glob Health, 8(4):e480 (2020). SARS-CoV-2 appears to bind to the ACE2 receptor which results in severe pneumonia and a high mortality rate in susceptible patients. See Sun et al, Zhonghua Jie He Hu Xi Za Zhi., 43(0):E014 (2020). Pharmaceutical therapy will be crucial to curtail and cure SARS-CoV-2 infections. Several vaccines against SARS-CoV-2 have been commercialized. Nonetheless, there is an urgent need for treatments that are specifically targeted to inhibit SARS-CoV-2 expression, replication, and infection for the treatment of COVID-19, either as a stand-alone therapy or in conjunction with a vaccine or other drugs. The disclosure is directed to this, as well as other, important ends.

### BRIEF SUMMARY

The disclosure provides nucleic acids (e.g., RNA, sense RNA, sense RNA, antisense RNA, siRNA), lipid nanoparticles comprising nucleic acids (e.g., RNA, sense RNA, sense RNA, antisense RNA, siRNA), pharmaceutical compositions comprising nucleic acids (e.g., RNA, sense RNA, sense RNA, antisense RNA, siRNA), and pharmaceutical compositions comprising lipid nanoparticles which comprise nucleic acids (e.g., RNA, sense RNA, sense RNA, antisense RNA, siRNA). The disclosure provides methods of treating COVID-19, SARS viruses (e.g., SARS-CoV, SARS-CoV-1, SARS-CoV-2, MERS-CoV), and Middle Eastern respiratory syndrome using nucleic acids (e.g., RNA, sense RNA, sense RNA, antisense RNA, siRNA), lipid nanoparticles, and pharmaceutical compositions. The disclosure provides drug delivery devices comprising nucleic acids (e.g., RNA, sense RNA, sense RNA, antisense RNA, siRNA), lipid nanoparticles, and pharmaceutical compositions. These and other embodiments and aspects of the disclosure are described herein.

### BRIEF DESCRIPTION OF THE DRAWINGS

FIGS. 1A-1C show the SARS-CoV-2 virus siRNA target sites and vector constructs. In FIG. 1A, the SARS-CoV-2 virus genome is shown schematically with the various siRNA target sites from the first screen. The non-infectious CoV-2 reporter vector (FIG. 1B) and the infectious CoV-1 reporter vector (FIG. 1C) are proposed for the in vitro and in vivo studies outlined in this disclosure. Upward arrows in FIGS. 1A-1C show the siRNA site.

FIG. 2 shows SARS-CoV-2 susceptibility to RNAi targeting. SARS-CoV-2 targeted siRNAs (as shown in Tables F and G) were transfected with a pSI-Check reporter vector with the 5′UTR or M protein target site cloned down stream of Renilla luciferase, and knockdown of luciferase activity of each siRNA was determined. The control is a GFP targeted siRNA. The average of triplicate treated 293HEK cells are shown with the standard deviation.

FIGS. 3A-3D provide hybridized siRNA. FIG. 3A is an siRNA capable of targeting the 5′UTR region of SARS-CoV-2, which comprises SEQ ID NO:2 hybridized to SEQ ID NO:4. FIG. 3B an siRNA capable of targeting the ‘5UTR/ORF1a region of SARS-CoV-2, which comprises SEQ ID NO:4 hybridized to SEQ ID NO:6. FIG. 3C is a negative control which comprises SEQ ID NO:8 hybridized to SEQ ID NO:10. FIG. 3D is the legend for the base-linkage-sugar modifications.

FIGS. 4A-4C show delivery of nanoparticles to lungs. FIG. 4A shows biodistribution of siRNA-loaded, DiR labeled liposome nanoparticles after intravenous (IV) injection in mice. Livers, spleens and lungs were removed 48 hours post IV administration and analyzed on the fluorescent imager (λex=720 nm; λem=790 nm). FIG. 4B shows quantitative analysis of fluorescence intensity for each individual organ. Results are the mean±SEM of n=6 from two independent experiments. FIG. 4C shows individual cell populations were analyzed by flow cytometry for DiR fluorescence. Delivery was quantified as a percentage of DiR+ cells/total cells in the specific population. Control was mice treated with unstained liposomes. Data shown are mean±SEM of n=6 mice per group analyzed in two independent experiments. These data have been adapted from previously published work (6).

FIG. 5 shows SiRNA effects. Delivery of lamin A/C siRNA (40 ug, i.v., 24 hours) into C57BL mice results in 88% knockdown of mRNA in lungs as measured by qPCR; and reduction in the expression of RSV N nucleoprotein gene with the range of RSV siRNAs.

FIGS. 6A-6G show SARS-CoV-2 susceptibility to RNAi targeting. FIG. 6A: a plaque assay 96 hours post-infection with COV-2 (0.0015 MOI). FIG. 6B: a plaque assay on the coronavirus directed ultra-conserved siRNA target sites. FIG. 6C: the dose dependent effect of the top-candidate siRNAs as determined by a plaque assay. For FIGS. 6A-6C the average of triplicate treated cells is shown from two separate experiments with the standard deviations shown and * representing statistical significance from an one-way ANOVA analysis (Dunnett's test). FIG. 6D: combinations of siRNAs efficiently repress CoV-2. Cells were transfected with indicated siRNAs (single or combined siRNAs at 30 nM final concentration) using Fugene 24 hours prior to infection with CoV-2 (250 PFU) and cultured for a further 96 hours after which a viral plaque assay was initiated. FIGS. 6E-6F: THP-1 DUAL cells were transfected with indicated siRNAs using Fugene 6 for 24 h before quantifying for IRF (FIG. 6E) and NFkB (FIG. 6F) gene reporter expression. Error bars denote SEM of triplicate treatments. 2′3′-cGAMP (20 μg/ml) and LPS (100 ng/ml) were used as positive controls for IRF and NFkB pathway stimulation, respectively. The siRNA UTR3 (siMod UTR3) are shown in FIG. 3A with the chemical modifications utilized to stabilize the siRNA and enhance persistence while inhibiting immunogenicity. FIG. 6G: the resultant unmodified siRNA controls and the modified siMod UTR3 were transfected with a pSI-Check reporter vector with the 5′UTR cloned down stream of Renilla luciferase, and knockdown of luciferase activity of the modified siRNA determined relative to the unmodified control. The average of triplicate treated 293HEK cells are shown with the standard deviation. The sequences for the siRNA shown in FIGS. 6A-6B are set forth in Tables D-F.

FIGS. 7A-7B show antisense non-coding RNA targeting of SARS-COV-2. FIG. 7A: screening antisense RNAs for repression of SARS-CoV-2. FIG. 7B: a repeat experiment was carried out focused on only antisense 600 bp and antisense 800 bp RNAs contrasted with the as600 and as800 controls targeted to HIV which is not present in the virus infected cells. For A-B cells in 96-well plates were transfected with 0.1 μg of the indicated plasmids using Fugene 6 for 24 hours prior to infection with 62.5 PFU of live SARS-CoV-2. An immune-plaque assay was carried out 24 hours post-infection to determine viral infectivity. The results from a single experiment of triplicate treated virus infected cells and controls are shown with the standard deviations.

### DETAILED DESCRIPTION

### Definitions

The abbreviations used herein have their conventional meaning within the chemical and biological arts. The chemical structures and formulae set forth herein are constructed according to the standard rules of chemical valency known in the chemical arts.

“SARS” refers to severe acute respiratory syndrome.

“SARS-CoV” refers to severe acute respiratory syndrome-associated coronavirus.

“SARS-CoV-1” refers to severe acute respiratory syndrome-associated coronavirus 1.

“SARS-CoV-2” refers to severe acute respiratory syndrome-associated coronavirus 2.

“COVID-19” refers to the disease caused by SARS-CoV-2. COVID-19 has an incubation period of 2-14 days, and symptoms include, e.g., fever, tiredness, cough, and shortness of breath (e.g., difficulty breathing).

“MERS-CoV” refers to Middle Eastern respiratory syndrome-associated coronavirus. See, e.g., Chung et al, Genetic Characterization of Middle East Respiratory Syndrome Coronavirus, South Korea, 2018. Emerging Infectious Diseases, 25(5):958-962 (2019).

“Middle Eastern respiratory syndrome” or “MERS” refers to the disease caused by MERS-CoV, and symptoms include, e.g., fever, cough, and shortness of breath (e.g., difficulty breathing).

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of nucleic acids contemplated herein include single and double stranded DNA, single and double stranded RNA, and hybrid molecules having mixtures of single and double stranded DNA and RNA. Examples of nucleic acids contemplated herein include any types of RNA (e.g., antisense RNA, mRNA, siRNA, miRNA, shRNA, guide RNA, dicer substrate RNA, dicer substrate siRNAs (dsiRNAs) (dsiRNA are cleaved by the RNase III class endoribonuclease dicer into 21-23 base duplexes having 2-base 3′-overhangs siRNA), and any type of DNA, genomic DNA, plasmid DNA, and minicircle DNA, and any fragments thereof. The term “duplex” in the context of nucleic acids refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as 2′O-methyl, 2′O-methoxyethoxy, 2′fluoro, 5-methyl cytidine or pseudouridine; and peptide nucleic acid backbones and linkages. Other analog nucleic acids include those with positive backbones; non-ionic backbones, modified sugars (e.g., deoxyribose), and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In aspects, the internucleotide linkages in DNA are phosphodiester, phosphodiester derivatives, or a combination of both.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

An “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In aspects, the antisense nucleic acid hybridizes to the target nucleic acid in vitro. In aspects, the antisense nucleic acid hybridizes to the target nucleic acid in a cell. In aspects, the antisense nucleic acid hybridizes to the target nucleic acid in an organism. In aspects, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and anomeric sugar-phosphate, backbone-modified nucleotides.

In the cell, the antisense nucleic acids hybridize to the corresponding RNA forming a double-stranded molecule. The antisense nucleic acids interfere with the endogenous behavior of the RNA and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include small interfering RNAs (siRNAs)(including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

“siRNA” and “small interfering RNA” as provided herein refers to a double-stranded or single-stranded ribonucleic acid that has the ability to reduce or inhibit expression of a gene or the activity of a target nucleic acid (e.g., a single-stranded or double-stranded RNA or a single-stranded or doubles-stranded DNA) when expressed in the same cell as the gene or target gene. Where the siRNA is a double-stranded RNA, the complementary portions of the ribonucleic acid that hybridize to form the double stranded molecule typically have substantial or complete identity. In aspects, an siRNA is a nucleic acid that has substantial or complete identity to a target RNA and forms a double stranded siRNA. In aspects, the siRNA inhibits gene expression by interacting with a complementary cellular RNA thereby interfering with the endogenous behavior of the complementary cellular RNA. Typically, the siRNA is about 15-50 nucleotides in length (e.g., each complementary sequence of the double stranded siRNA is 15-50 nucleotides in length, and the double stranded siRNA is about 15-50 base pairs in length). The siRNAs provided herein regulate expression of a target gene or activity of a target nucleic by hybridizing to the mRNA of the gene or by hybridizing to the promoter of the target nucleic or the target nucleic acid itself. Where the siRNA hybridizes to a promoter of a gene thereby modulating the expression of said gene, the siRNA may be referred to as “antigen RNA” or “agRNA.” In aspects, the nucleic acid sequences provided herein are siRNA.

The terms “short hairpin RNA” or “shRNA” refer to an anti-sense ribonucleic acid sequence as defined above, which is capable of binding (hybridizing) and inhibiting activity of a target RNA (e.g., mRNA), which it is partially or entirely complementary to. shRNA may be single stranded nucleic acid sequences having a secondary or tertiary structure and, thus, may be able to fold into diverse and intricate molecular structures such as stem-loop structures.

“Dicer-substrate small interfering RNA” or “dsiRNA” are RNA duplexes that are processed by the Dicer enzyme into 25 to 30 nucleotides in length, and are effective triggers of RNA interference.

“Hybridize” and “hybridization” refer to the pairing of complementary (including partially complementary) nucleic acid strands. Hybridization and the strength of hybridization (e.g., the strength of the association between nucleic acid strands) is impacted by factors known in the art including the degree of complementarity between the nucleic acid, stringency of the conditions involved affected by such conditions as the concentration of salts, the melting temperature (Tm) of the formed hybrid, the presence of other components, the molarity of the hybridizing strands and the G:C content of the nucleic acid strands. When one nucleic acid is said to “hybridize” to another nucleic acid, it means that there is some complementarity between the two nucleic acids or that the two nucleic acids form a hybrid under high or low stringency conditions.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The phrase “hybridization conditions” refers to conditions under which a nucleic acid will hybridize to its target sequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at T, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary hybridization conditions can be as follows: 50% formamide, 5×SSC, and 1% SDS, incubating at 42° C., or 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For PCR amplification, a temperature of about 62° C. is typical, although high stringency annealing temperatures can range from about 50° C. to about 65° C. depending on the primer length and specificity. Typical cycle conditions for both high and low stringency amplifications include a denaturation phase of 90° C.-95° C. for 30 seconds to 2 minutes, an annealing phase lasting 30 seconds to 2 minutes, and an extension phase of about 72° C. for 1-2 min. Protocols and guidelines for low and high stringency amplification reactions are provided, e.g., in Innis et al., PCR Protocols, A Guide to Methods and Applications, Academic Press, Inc. N.Y. (1990).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

The terms “polypeptide,” “peptide” and “protein” are used interchangeably herein to refer to a polymer of amino acid residues, wherein the polymer may In aspects be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (e.g., http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In aspects, the nucleic acids described herein are isolated nucleic acids.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

The term “activation”, “activate”, “activating”, “activator” and the like in reference to a protein-inhibitor interaction means positively affecting (e.g. increasing) the activity or function of the protein relative to the activity or function of the protein in the absence of the activator. In aspects activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

The term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In aspects inhibition means negatively affecting (e.g. decreasing) the concentration or levels of the protein relative to the concentration or level of the protein in the absence of the inhibitor. In aspects inhibition refers to reduction of a disease or symptoms of disease. In aspects, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In aspects, inhibition refers to a reduction of activity of a target protein resulting from a direct interaction (e.g. an inhibitor binds to the target protein). In aspects, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “target gene” refers to any nucleic acid sequence which contains an identified genes or a target region within a gene, including intergenic regions, non-coding regions, untranscribed regions, introns, exons, and transgenes. The target gene (or a target site within the gene) can be a gene derived from a cell, an endogenous gene, a transgene, or exogenous genes such as genes of a pathogen, for example a virus, which is present in the cell after infection thereof. The cell containing the target gene can be derived from or contained in any organism.

The terms “treating” or “treatment” refers to any indicia of success in the therapy or amelioration of a disease, pathology or condition, including any objective or subjective parameter such as abatement; remission; diminishing of symptoms or making the pathology or condition more tolerable to the patient; slowing in the rate of degeneration or decline; making the final point of degeneration less debilitating; improving a patient's physical well-being. The treatment or amelioration of symptoms can be based on objective or subjective parameters; including the results of a physical examination. The term “treating” and conjugations thereof, may include prevention of a pathology, condition, or disease. In aspects, treating is preventing. In aspects, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent (e.g., nucleic acids, antisense RNA, siRNA). The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

“Patient” or “subject in need thereof” refers to a living organism. Non-limiting examples include humans, other mammals, bovines, rats, mice, dogs, monkeys, goat, sheep, cows, and other non-mammalian animals. In aspects, a patient is human.

A “effective amount,” as used herein, is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). In these methods, the effective amount of the nucleic acid (antisense RNA, siRNA) described herein is an amount effective to accomplish the stated purpose of the method. An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. For any compound described herein, the therapeutically effective amount can be initially determined from cell culture assays. Target concentrations will be those concentrations of active compound(s) that are capable of achieving the methods described herein, as measured using the methods described herein or known in the art. As is in the art, therapeutically effective amounts for use in humans can also be determined from animal models. For example, a dose for humans can be formulated to achieve a concentration that has been found to be effective in animals.

The term “administering” means intranasal administration, inhalation administration, oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include the use of lipid nanoparticles, aerosols, liposomal formulations, intravenous infusion, transdermal patches, and the like. In aspects, administering does not include administration of any active agent other than the nucleic acid. In aspects, administration is intranasal. In aspects, administration is intravenous. In aspects, administration is intranasal administration of lipid nanoparticles. In aspects, administration is intravenous administration of lipid nanoparticles.

Nucleic Acids

The disclosure provides nucleic acids comprising at least 10 nucleotides and capable of hybridizing to a 5′UTR region or a 5′UTR/ORF1a region in a SARS-associated coronavirus. In aspects, the 5′UTR region or the 5′UTR/ORF1a region spans nucleotides 1 to 447 in the SARS-associated coronavirus. In aspects, the SARS-associated coronavirus is SARS-associated coronavirus 2. In aspects, the 5′UTR region or the 5′UTR/ORF1a region of the SARS-associated coronavirus 2 comprises the sequence of any one of SEQ ID NOS:29-49. In aspects, the nucleic acids comprise at least 10 nucleotides and capable of hybridizing to a 5′UTR region or a 5′UTR/ORF1a region in a SARS-associated coronavirus 2 is a nucleic acid capable of hybridizing to a nucleotide sequence selected from the group consisting of SEQ ID NOS:13-20 and 26-28. In aspects, the nucleic acid is capable of hybridizing to SEQ ID NO:13. In aspects, the nucleic acid is capable of hybridizing to SEQ ID NO:14. In aspects, the nucleic acid is capable of hybridizing to SEQ ID NO:15. In aspects, the nucleic acid is capable of hybridizing to SEQ ID NO:16. In aspects, the nucleic acid is capable of hybridizing to SEQ ID NO:17. In aspects, the nucleic acid is capable of hybridizing to SEQ ID NO:18. In aspects, the nucleic acid is capable of hybridizing to SEQ ID NO:19. In aspects, the nucleic acid is capable of hybridizing to SEQ ID NO:20. In aspects, the nucleic acid is capable of hybridizing to SEQ ID NO:26. In aspects, the nucleic acid is capable of hybridizing to SEQ ID NO:27. In aspects, the nucleic acid is capable of hybridizing to SEQ ID NO:28. In aspects, the nucleic acid is RNA. In aspects, the nucleic acid is antisense RNA. In aspects, the nucleic acid is siRNA.

The disclosure provides nucleic acids comprising at least 10 nucleotides and capable of hybridizing to a 5′UTR region or a 5′UTR/ORF1a region in a SARS-associated coronavirus. In aspects, the 5′UTR region or the 5′UTR/ORF1a region spans nucleotides 1 to 447 in the SARS-associated coronavirus. In aspects, the SARS-associated coronavirus is SARS-associated coronavirus 1. In aspects, the nucleic acids comprise at least 10 nucleotides and capable of hybridizing to a 5′UTR region or a 5′UTR/ORF1a region in a SARS-associated coronavirus 1 is a nucleic acid capable of hybridizing to SEQ ID NO:50. In aspects, the nucleic acids comprising at least 10 nucleotides and capable of hybridizing to a 5′UTR region or a 5′UTR/ORF1a region in a SARS-associated coronavirus 1 is a nucleic acid capable of hybridizing to a nucleotide sequence of SEQ ID NO:51, SEQ ID NO:52, or SEQ ID NO:53. In aspects, the nucleic acid is RNA. In aspects, the nucleic acid is antisense RNA. In aspects, the nucleic acid is siRNA.

In aspects, the disclosure provides nucleic acids comprising at least 10 nucleotides and capable of hybridizing to a 5′UTR region or a 5′UTR/ORF1a region in a SARS-associated coronavirus. In aspects, the nucleic acids comprise about 15 or more nucleotides. In aspects, the nucleic acids comprise at least 19 nucleotides. In aspects, the nucleic acids comprise at 20 or more nucleotides. In aspects, the nucleic acids comprise at least 20 nucleotides. In aspects, the nucleic acids comprise from about 15 nucleotides to about 25 nucleotides. In aspects, the nucleic acids comprise from about 20 nucleotides to about 25 nucleotides. In aspects, the nucleic acids comprise from about 21 nucleotides to about 23 nucleotides. In aspects, the nucleic acids comprise 20 nucleotides. In aspects, the nucleic acids comprise 21 nucleotides. In aspects, the nucleic acids comprise 22 nucleotides. In aspects, the nucleic acids comprise 23 nucleotides. In aspects, the nucleic acids comprise 24 nucleotides. In aspects, the nucleic acids comprise 25 nucleotides. In aspects, one or more nucleotides in the nucleic acids comprise a modified base, a modified sugar, a modified phosphate, or a combination of two or more thereof. In aspects, the modified base is a 2′O-Methyl modified base, a 2′O-methoxyethoxy modified base, a 2′fluoro modified base, a 5-methyl-cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose.

The disclosure provides nucleic acids comprising at least 10 nucleotides and capable of hybridizing to an essential membrane protein (M) region in a SARS-associated coronavirus. In aspects, the essential membrane (M) region spans nucleotides 26519 to 27222 in the SARS-associated coronavirus. In aspects, the essential membrane (M) region spans nucleotides 26552 to 27222 in the SARS-associated coronavirus. In aspects, the SARS-associated coronavirus is SARS-associated coronavirus 2. In aspects, the essential membrane protein (M) region of the SARS-associated coronavirus 2 comprises the sequence of any one of SEQ ID NOS:57-77. In aspects, the nucleic acids comprise at least 10 nucleotides and capable of hybridizing to an essential membrane protein (M) region in a SARS-associated coronavirus 2 is a nucleic acid capable of hybridizing to a nucleotide sequence selected from the group consisting of SEQ ID NOS:21-25 and 54-56. In aspects, the nucleic acid is capable of hybridizing to SEQ ID NO:21. In aspects, the nucleic acid is capable of hybridizing to SEQ ID NO:22. In aspects, the nucleic acid is capable of hybridizing to SEQ ID NO:23. In aspects, the nucleic acid is capable of hybridizing to SEQ ID NO:24. In aspects, the nucleic acid is capable of hybridizing to SEQ ID NO:25. In aspects, the nucleic acid is capable of hybridizing to SEQ ID NO:54. In aspects, the nucleic acid is capable of hybridizing to SEQ ID NO:55. In aspects, the nucleic acid is capable of hybridizing to SEQ ID NO:56. In aspects, the nucleic acid is RNA. In aspects, the nucleic acid is antisense RNA. In aspects, the nucleic acid is siRNA.

The disclosure provides nucleic acids comprising at least 10 nucleotides and capable of hybridizing to an essential membrane protein (M) region in a SARS-associated coronavirus. In aspects, the essential membrane (M) region spans nucleotides 26519 to 27222 in the SARS-associated coronavirus. In aspects, the essential membrane (M) region spans nucleotides 26552 to 27222 in the SARS-associated coronavirus. In aspects, the SARS-associated coronavirus is SARS-associated coronavirus 1. In aspects, the essential membrane protein (M) region of the SARS-associated coronavirus 1 comprises the sequence of SEQ ID NO:78. In aspects, the nucleic acid is capable of hybridizing to a nucleotide sequence selected from the group consisting of SEQ ID NOS: 79-81. In aspects, the nucleic acid is capable of hybridizing to SEQ ID NO:79. In aspects, the nucleic acid is capable of hybridizing to SEQ ID NO:80. In aspects, the nucleic acid is capable of hybridizing to SEQ ID NO:81. In aspects, the nucleic acid is RNA. In aspects, the nucleic acid is antisense RNA. In aspects, the nucleic acid is siRNA.

In aspects, the disclosure provides nucleic acids comprising at least 10 nucleotides and capable of hybridizing to an essential membrane protein (M) region in a SARS-associated coronavirus. In aspects, the nucleic acids comprise at least 19 nucleotides. In aspects, the nucleic acids comprise about 20 or more nucleotides. In aspects, the nucleic acids comprise at least 20 nucleotides. In aspects, the nucleic acids comprise from about 15 nucleotides to about 25 nucleotides. In aspects, the nucleic acids comprise from about 20 nucleotides to about 25 nucleotides. In aspects, the nucleic acids comprise from about 21 nucleotides to about 23 nucleotides. In aspects, the nucleic acids comprise 20 nucleotides. In aspects, the nucleic acids comprise 21 nucleotides. In aspects, the nucleic acids comprise 22 nucleotides. In aspects, the nucleic acids comprise 23 nucleotides. In aspects, the nucleic acids comprise 24 nucleotides. In aspects, the nucleic acids comprise 25 nucleotides. In aspects, one or more nucleotides in the nucleic acids comprise a modified base, a modified sugar, a modified phosphate, or a combination of two or more thereof. In aspects, the modified base is a 2′O-Methyl modified base, a 2′O-methoxyethoxy modified base, a 2′fluoro modified base, a 5-methyl-cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose.

In aspects, the disclosure provides nucleic acids comprising at least 10 nucleotides and capable of hybridizing to any one of SEQ ID NOS:100-124, 223-235, or 258-265. In aspects, the nucleic acid is capable of hybridizing to SEQ ID NO:100. In aspects, the nucleic acid is capable of hybridizing to SEQ ID NO:101. In aspects, the nucleic acid is capable of hybridizing to SEQ ID NO:102. In aspects, the nucleic acid is capable of hybridizing to SEQ ID NO:103. In aspects, the nucleic acid is capable of hybridizing to SEQ ID NO:104. In aspects, the nucleic acid is capable of hybridizing to SEQ ID NO:105. In aspects, the nucleic acid is capable of hybridizing to SEQ ID NO:106. In aspects, the nucleic acid is capable of hybridizing to SEQ ID NO:107. In aspects, the nucleic acid is capable of hybridizing to SEQ ID NO:108. In aspects, the nucleic acid is capable of hybridizing to SEQ ID NO:109. In aspects, the nucleic acid is capable of hybridizing to SEQ ID NO:110. In aspects, the nucleic acid is capable of hybridizing to SEQ ID NO:111. In aspects, the nucleic acid is capable of hybridizing to SEQ ID NO:112. In aspects, the nucleic acid is capable of hybridizing to SEQ ID NO:113. In aspects, the nucleic acid is capable of hybridizing to SEQ ID NO:114. In aspects, the nucleic acid is capable of hybridizing to SEQ ID NO:115. In aspects, the nucleic acid is capable of hybridizing to SEQ ID NO:116. In aspects, the nucleic acid is capable of hybridizing to SEQ ID NO:117. In aspects, the nucleic acid is capable of hybridizing to SEQ ID NO:118. In aspects, the nucleic acid is capable of hybridizing to SEQ ID NO:119. In aspects, the nucleic acid is capable of hybridizing to SEQ ID NO:120. In aspects, the nucleic acid is capable of hybridizing to SEQ ID NO:121. In aspects, the nucleic acid is capable of hybridizing to SEQ ID NO:122. In aspects, the nucleic acid is capable of hybridizing to SEQ ID NO:123. In aspects, the nucleic acid is capable of hybridizing to SEQ ID NO:124. In aspects, the nucleic acid is capable of hybridizing to SEQ ID NO:223. In aspects, the nucleic acid is capable of hybridizing to SEQ ID NO:224. In aspects, the nucleic acid is capable of hybridizing to SEQ ID NO:225. In aspects, the nucleic acid is capable of hybridizing to SEQ ID NO:226. In aspects, the nucleic acid is capable of hybridizing to SEQ ID NO:227. In aspects, the nucleic acid is capable of hybridizing to SEQ ID NO:228. In aspects, the nucleic acid is capable of hybridizing to SEQ ID NO:229. In aspects, the nucleic acid is capable of hybridizing to SEQ ID NO:230. In aspects, the nucleic acid is capable of hybridizing to SEQ ID NO:231. In aspects, the nucleic acid is capable of hybridizing to SEQ ID NO:232. In aspects, the nucleic acid is capable of hybridizing to SEQ ID NO:233. In aspects, the nucleic acid is capable of hybridizing to SEQ ID NO:234. In aspects, the nucleic acid is capable of hybridizing to SEQ ID NO:235. In aspects, the nucleic acid is capable of hybridizing to SEQ ID NO:258. In aspects, the nucleic acid is capable of hybridizing to SEQ ID NO:259. In aspects, the nucleic acid is capable of hybridizing to SEQ ID NO:260. In aspects, the nucleic acid is capable of hybridizing to SEQ ID NO:261. In aspects, the nucleic acid is capable of hybridizing to SEQ ID NO:262. In aspects, the nucleic acid is capable of hybridizing to SEQ ID NO:263. In aspects, the nucleic acid is capable of hybridizing to SEQ ID NO:264. In aspects, the nucleic acid is capable of hybridizing to SEQ ID NO:265. In aspects, the nucleic acids comprise at least 19 nucleotides. In aspects, the nucleic acids comprise about 20 or more nucleotides. In aspects, the nucleic acids comprise at least 20 nucleotides. In aspects, the nucleic acids comprise from about 15 nucleotides to about 25 nucleotides. In aspects, the nucleic acids comprise from about 20 nucleotides to about 25 nucleotides. In aspects, the nucleic acids comprise from about 21 nucleotides to about 23 nucleotides. In aspects, the nucleic acids comprise 20 nucleotides. In aspects, the nucleic acids comprise 21 nucleotides. In aspects, the nucleic acids comprise 22 nucleotides. In aspects, the nucleic acids comprise 23 nucleotides. In aspects, the nucleic acids comprise 24 nucleotides. In aspects, the nucleic acids comprise 25 nucleotides. In aspects, one or more nucleotides in the nucleic acids comprise a modified base, a modified sugar, a modified phosphate, or a combination of two or more thereof. In aspects, the modified base is a 2′O-Methyl modified base, a 2′O-methoxyethoxy modified base, a 2′fluoro modified base, a 5-methyl-cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose.

In aspects, the disclosure provides nucleic acids comprising at least 10 nucleotides and capable of hybridizing to SEQ ID NO:224, SEQ ID NO:231, SEQ ID NO:124, SEQ ID NO:223, SEQ ID NO:225, SEQ ID NO:230, or SEQ ID NO:123. In aspects, the nucleic acid is capable of hybridizing to SEQ ID NO:224. In aspects, the nucleic acid is capable of hybridizing to SEQ ID NO:231. In aspects, the nucleic acid is capable of hybridizing to SEQ ID NO:124. In aspects, the nucleic acid is capable of hybridizing to SEQ ID NO:223. In aspects, the nucleic acid is capable of hybridizing to SEQ ID NO:225. In aspects, the nucleic acid is capable of hybridizing to SEQ ID NO:230. In aspects, the nucleic acid is capable of hybridizing to SEQ ID NO:123. In aspects, the nucleic acids comprise at least 19 nucleotides. In aspects, the nucleic acids comprise about 20 or more nucleotides. In aspects, the nucleic acids comprise at least 20 nucleotides. In aspects, the nucleic acids comprise from about 15 nucleotides to about 25 nucleotides. In aspects, the nucleic acids comprise from about 20 nucleotides to about 25 nucleotides. In aspects, the nucleic acids comprise from about 21 nucleotides to about 23 nucleotides. In aspects, the nucleic acids comprise 20 nucleotides. In aspects, the nucleic acids comprise 21 nucleotides. In aspects, the nucleic acids comprise 22 nucleotides. In aspects, the nucleic acids comprise 23 nucleotides. In aspects, the nucleic acids comprise 24 nucleotides. In aspects, the nucleic acids comprise 25 nucleotides. In aspects, one or more nucleotides in the nucleic acids comprise a modified base, a modified sugar, a modified phosphate, or a combination of two or more thereof. In aspects, the modified base is a 2′O-Methyl modified base, a 2′O-methoxyethoxy modified base, a 2′fluoro modified base, a 5-methyl-cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose.

In aspects, the disclosure provides a nucleic acid comprising SEQ ID NO:1. In aspects, the disclosure provides a nucleic acid comprising SEQ ID NO:3. In aspects, the disclosure provides a nucleic acid comprising SEQ ID NO:1 hybridized to SEQ ID NO:3. In aspects, the nucleic acid of SEQ ID NO:1 comprises: (i) at least one modified base, (ii) at least one modified sugar, (iii) at least one modified phosphate, or (iv) any combination of the foregoing. In aspects, the nucleic acid of SEQ ID NO:3 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5-methyl-cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a base modified with 2′O-Methyl. In aspects, the modified phosphate is phosphorothioate. In aspects, the modified sugar is deoxyribose. In aspects, the nucleic acid of SEQ ID NO:1 is a modified nucleic acid comprising SEQ ID NO:2. In aspects, the nucleic acid of SEQ ID NO:1 is a modified nucleic acid of SEQ ID NO:2. In aspects, the nucleic acid of SEQ ID NO:3 is a modified nucleic acid comprising SEQ ID NO:4. In aspects, the nucleic acid of SEQ ID NO:3 is a modified nucleic acid of SEQ ID NO:4. In aspects, the disclosure provides a nucleic acid comprising SEQ ID NO:2 hybridized to SEQ ID NO:4. In aspects, the nucleic acid is RNA. In aspects, the nucleic acid is antisense RNA. In aspects, the nucleic acid is siRNA.

In aspects, the disclosure provides a nucleic acid comprising SEQ ID NO:5. In aspects, the disclosure provides a nucleic acid comprising SEQ ID NO:7. In aspects, the disclosure provides a nucleic acid comprising SEQ ID NO:5 hybridized to SEQ ID NO:7. In aspects, the nucleic acid of SEQ ID NO:5 comprises: (i) at least one modified base, (ii) at least one modified sugar, (iii) at least one modified phosphate, or (iv) any combination of the foregoing. In aspects, the nucleic acid of SEQ ID NO:7 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5-methyl-cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid of SEQ ID NO:5 is a modified nucleic acid comprising SEQ ID NO:6. In aspects, the nucleic acid of SEQ ID NO:5 is a modified nucleic acid of SEQ ID NO:6. In aspects, the nucleic acid of SEQ ID NO:7 is a modified nucleic acid comprising SEQ ID NO:8. In aspects, the nucleic acid of SEQ ID NO:7 is a modified nucleic acid of SEQ ID NO:8. In aspects, the disclosure provides a nucleic acid comprising SEQ ID NO:6 hybridized to SEQ ID NO:8. In aspects, the nucleic acid is RNA. In aspects, the nucleic acid is antisense RNA. In aspects, the nucleic acid is siRNA.

In aspects, the disclosure provides a nucleic acid comprising SEQ ID NO:82. In aspects, the disclosure provides a nucleic acid comprising SEQ ID NO:83. In aspects, the disclosure provides a nucleic acid comprising SEQ ID NO:82 hybridized to SEQ ID NO:83. In aspects, the nucleic acid of SEQ ID NO:82 comprises: (i) at least one modified base, (ii) at least one modified sugar, (iii) at least one modified phosphate, or (iv) any combination of the foregoing. In aspects, the nucleic acid of SEQ ID NO:83 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:84. In aspects, the disclosure provides a nucleic acid comprising SEQ ID NO:85. In aspects, the disclosure provides a nucleic acid comprising SEQ ID NO:84 hybridized to SEQ ID NO:85. In aspects, the nucleic acid of SEQ ID NO:84 comprises: (i) at least one modified base, (ii) at least one modified sugar, (iii) at least one modified phosphate, or (iv) any combination of the foregoing. In aspects, the nucleic acid of SEQ ID NO:85 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:86. In aspects, the disclosure provides a nucleic acid comprising SEQ ID NO: 87. In aspects, the disclosure provides a nucleic acid comprising SEQ ID NO:86 hybridized to SEQ ID NO:87. In aspects, the nucleic acid of SEQ ID NO:86 comprises: (i) at least one modified base, (ii) at least one modified sugar, (iii) at least one modified phosphate, or (iv) any combination of the foregoing. In aspects, the nucleic acid of SEQ ID NO:87 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:88. In aspects, the disclosure provides a nucleic acid comprising SEQ ID NO: 89. In aspects, the disclosure provides a nucleic acid comprising SEQ ID NO:88 hybridized to SEQ ID NO:89. In aspects, the nucleic acid of SEQ ID NO:88 comprises: (i) at least one modified base, (ii) at least one modified sugar, (iii) at least one modified phosphate, or (iv) any combination of the foregoing. In aspects, the nucleic acid of SEQ ID NO:89 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:90. In aspects, the disclosure provides a nucleic acid comprising SEQ ID NO:91. In aspects, the disclosure provides a nucleic acid comprising SEQ ID NO:90 hybridized to SEQ ID NO:91. In aspects, the nucleic acid of SEQ ID NO:90 comprises: (i) at least one modified base, (ii) at least one modified sugar, (iii) at least one modified phosphate, or (iv) any combination of the foregoing. In aspects, the nucleic acid of SEQ ID NO:91 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:92. In aspects, the disclosure provides a nucleic acid comprising SEQ ID NO:93. In aspects, the disclosure provides a nucleic acid comprising SEQ ID NO:92 hybridized to SEQ ID NO:93. In aspects, the nucleic acid of SEQ ID NO:92 comprises: (i) at least one modified base, (ii) at least one modified sugar, (iii) at least one modified phosphate, or (iv) any combination of the foregoing. In aspects, the nucleic acid of SEQ ID NO:93 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:94. In aspects, the disclosure provides a nucleic acid comprising SEQ ID NO:95. In aspects, the disclosure provides a nucleic acid comprising SEQ ID NO:94 hybridized to SEQ ID NO:95. In aspects, the nucleic acid of SEQ ID NO:94 comprises: (i) at least one modified base, (ii) at least one modified sugar, (iii) at least one modified phosphate, or (iv) any combination of the foregoing. In aspects, the modified sugar is deoxyribose. In aspects, the nucleic acid of SEQ ID NO:95 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:96. In aspects, the disclosure provides a nucleic acid comprising SEQ ID NO:97. In aspects, the disclosure provides a nucleic acid comprising SEQ ID NO:96 hybridized to SEQ ID NO:97. In aspects, the nucleic acid of SEQ ID NO:96 comprises: (i) at least one modified base, (ii) at least one modified sugar, (iii) at least one modified phosphate, or (iv) any combination of the foregoing. In aspects, the nucleic acid of SEQ ID NO:97 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising a sense strand in Table A. In aspects, the disclosure provides a nucleic acid comprising an antisense strand in Table A. In aspects, the disclosure provides a nucleic acid comprising a sense strand in Table A hybridized to the complementary antisense strand in Table A. In aspects, the nucleic acid comprising a sense strand in Table A comprises: (i) at least one modified base, (ii) at least one modified sugar, (iii) at least one modified phosphate, or (iv) any combination of the foregoing. In aspects, the nucleic acid comprising an antisense strand in Table A comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose.

In aspects, the disclosure provides a nucleic acid comprising a sense strand in Table B. In aspects, the disclosure provides a nucleic acid comprising an antisense strand in Table B. In aspects, the disclosure provides a nucleic acid comprising a sense strand in Table B hybridized to the complementary antisense strand in Table B. In aspects, the nucleic acid comprising a sense strand in Table B comprises: (i) at least one modified base, (ii) at least one modified sugar, (iii) at least one modified phosphate, or (iv) any combination of the foregoing. In aspects, the nucleic acid comprising an antisense strand in Table B comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose.

In aspects, the disclosure provides a nucleic acid comprising a sense strand in Table C. In aspects, the disclosure provides a nucleic acid comprising an antisense strand in Table C. In aspects, the disclosure provides a nucleic acid comprising a sense strand in Table C hybridized to the complementary antisense strand in Table C. In aspects, the nucleic acid comprising a sense strand in Table C comprises: (i) at least one modified base, (ii) at least one modified sugar, (iii) at least one modified phosphate, or (iv) any combination of the foregoing. In aspects, the nucleic acid comprising an antisense strand in Table C comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose.

In aspects, the disclosure provides a nucleic acid comprising a sense strand in Table D. In aspects, the disclosure provides a nucleic acid comprising an antisense strand in Table D. In aspects, the disclosure provides a nucleic acid comprising a sense strand in Table D hybridized to the complementary antisense strand in Table D. In aspects, the nucleic acid comprising a sense strand in Table D comprises: (i) at least one modified base, (ii) at least one modified sugar, (iii) at least one modified phosphate, or (iv) any combination of the foregoing. In aspects, the nucleic acid comprising an antisense strand in Table D comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose.

In aspects, the disclosure provides a nucleic acid comprising SEQ ID NO:214. In aspects, the disclosure provides a nucleic acid comprising SEQ ID NO:215. In aspects, the disclosure provides a nucleic acid comprising SEQ ID NO:214 hybridized to SEQ ID NO:215. In aspects, the nucleic acid of SEQ ID NO:214 comprises: (i) at least one modified base, (ii) at least one modified sugar, (iii) at least one modified phosphate, or (iv) any combination of the foregoing. In aspects, the nucleic acid of SEQ ID NO:215 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:216. In aspects, the disclosure provides a nucleic acid comprising SEQ ID NO:217. In aspects, the disclosure provides a nucleic acid comprising SEQ ID NO:216 hybridized to SEQ ID NO:217. In aspects, the nucleic acid of SEQ ID NO:216 comprises: (i) at least one modified base, (ii) at least one modified sugar, (iii) at least one modified phosphate, or (iv) any combination of the foregoing. In aspects, the nucleic acid of SEQ ID NO:217 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:218. In aspects, the disclosure provides a nucleic acid comprising SEQ ID NO:219. In aspects, the disclosure provides a nucleic acid comprising SEQ ID NO:218 hybridized to SEQ ID NO:219. In aspects, the nucleic acid of SEQ ID NO:218 comprises: (i) at least one modified base, (ii) at least one modified sugar, (iii) at least one modified phosphate, or (iv) any combination of the foregoing. In aspects, the nucleic acid of SEQ ID NO:219 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:220. In aspects, the disclosure provides a nucleic acid comprising SEQ ID NO:221. In aspects, the disclosure provides a nucleic acid comprising SEQ ID NO:220 hybridized to SEQ ID NO:221. In aspects, the nucleic acid of SEQ ID NO:220 comprises: (i) at least one modified base, (ii) at least one modified sugar, (iii) at least one modified phosphate, or (iv) any combination of the foregoing. In aspects, the nucleic acid of SEQ ID NO:221 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:238. In aspects, the disclosure provides a nucleic acid comprising SEQ ID NO:239. In aspects, the disclosure provides a nucleic acid comprising SEQ ID NO:238 hybridized to SEQ ID NO:239. In aspects, the nucleic acid of SEQ ID NO:238 comprises: (i) at least one modified base, (ii) at least one modified sugar, (iii) at least one modified phosphate, or (iv) any combination of the foregoing. In aspects, the nucleic acid of SEQ ID NO:239 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:240. In aspects, the disclosure provides a nucleic acid comprising SEQ ID NO:241. In aspects, the disclosure provides a nucleic acid comprising SEQ ID NO:240 hybridized to SEQ ID NO:241. In aspects, the nucleic acid of SEQ ID NO:240 comprises: (i) at least one modified base, (ii) at least one modified sugar, (iii) at least one modified phosphate, or (iv) any combination of the foregoing. In aspects, the nucleic acid of SEQ ID NO:241 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:242. In aspects, the disclosure provides a nucleic acid comprising SEQ ID NO:243. In aspects, the disclosure provides a nucleic acid comprising SEQ ID NO:242 hybridized to SEQ ID NO:243. In aspects, the nucleic acid of SEQ ID NO:242 comprises: (i) at least one modified base, (ii) at least one modified sugar, (iii) at least one modified phosphate, or (iv) any combination of the foregoing. In aspects, the nucleic acid of SEQ ID NO:243 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:244. In aspects, the disclosure provides a nucleic acid comprising SEQ ID NO:245. In aspects, the disclosure provides a nucleic acid comprising SEQ ID NO:244 hybridized to SEQ ID NO:245. In aspects, the nucleic acid of SEQ ID NO:244 comprises: (i) at least one modified base, (ii) at least one modified sugar, (iii) at least one modified phosphate, or (iv) any combination of the foregoing. In aspects, the nucleic acid of SEQ ID NO:245 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:246. In aspects, the disclosure provides a nucleic acid comprising SEQ ID NO:247. In aspects, the disclosure provides a nucleic acid comprising SEQ ID NO:246 hybridized to SEQ ID NO:247. In aspects, the nucleic acid of SEQ ID NO:246 comprises: (i) at least one modified base, (ii) at least one modified sugar, (iii) at least one modified phosphate, or (iv) any combination of the foregoing. In aspects, the nucleic acid of SEQ ID NO:247 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:248. In aspects, the disclosure provides a nucleic acid comprising SEQ ID NO:249. In aspects, the disclosure provides a nucleic acid comprising SEQ ID NO:248 hybridized to SEQ ID NO:249. In aspects, the nucleic acid of SEQ ID NO:248 comprises: (i) at least one modified base, (ii) at least one modified sugar, (iii) at least one modified phosphate, or (iv) any combination of the foregoing. In aspects, the nucleic acid of SEQ ID NO:249 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:250. In aspects, the disclosure provides a nucleic acid comprising SEQ ID NO:251. In aspects, the disclosure provides a nucleic acid comprising SEQ ID NO:250 hybridized to SEQ ID NO:251. In aspects, the nucleic acid of SEQ ID NO:250 comprises: (i) at least one modified base, (ii) at least one modified sugar, (iii) at least one modified phosphate, or (iv) any combination of the foregoing. In aspects, the nucleic acid of SEQ ID NO:251 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:252. In aspects, the disclosure provides a nucleic acid comprising SEQ ID NO:253. In aspects, the disclosure provides a nucleic acid comprising SEQ ID NO:252 hybridized to SEQ ID NO:253. In aspects, the nucleic acid of SEQ ID NO:240 comprises: (i) at least one modified base, (ii) at least one modified sugar, (iii) at least one modified phosphate, or (iv) any combination of the foregoing. In aspects, the nucleic acid of SEQ ID NO:241 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:254. In aspects, the disclosure provides a nucleic acid comprising SEQ ID NO:255. In aspects, the disclosure provides a nucleic acid comprising SEQ ID NO:254 hybridized to SEQ ID NO:255. In aspects, the nucleic acid of SEQ ID NO:254 comprises: (i) at least one modified base, (ii) at least one modified sugar, (iii) at least one modified phosphate, or (iv) any combination of the foregoing. In aspects, the nucleic acid of SEQ ID NO:255 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In aspects, the disclosure provides a nucleic acid comprising SEQ ID NO:256. In aspects, the disclosure provides a nucleic acid comprising SEQ ID NO:257. In aspects, the disclosure provides a nucleic acid comprising SEQ ID NO:256 hybridized to SEQ ID NO:257. In aspects, the nucleic acid of SEQ ID NO:256 comprises: (i) at least one modified base, (ii) at least one modified sugar, (iii) at least one modified phosphate, or (iv) any combination of the foregoing. In aspects, the nucleic acid of SEQ ID NO:257 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

The disclosure provides nucleic acids having at least 75% sequence identity to the nucleic acid sequence of any one of SEQ ID NOS:1-8 and 82-97. The disclosure provides nucleic acids having at least 80% sequence identity to the nucleic acid sequence of any one of SEQ ID NOS:1-8 and 82-97. The disclosure provides nucleic acids having at least 85% sequence identity to the nucleic acid sequence of any one of SEQ ID NOS:1-8 and 82-97. The disclosure provides nucleic acids having at least 90% sequence identity to the nucleic acid sequence of any one of SEQ ID NOS:1-8 and 82-97. The disclosure provides nucleic acids having at least 91% sequence identity to the nucleic acid sequence of any one of SEQ ID NOS:1-8 and 82-97. The disclosure provides nucleic acids having at least 92% sequence identity to the nucleic acid sequence of any one of SEQ ID NOS:1-8 and 82-97. The disclosure provides nucleic acids having at least 93% sequence identity to the nucleic acid sequence of any one of SEQ ID NOS:1-8 and 82-97. The disclosure provides nucleic acids having at least 94% sequence identity to the nucleic acid sequence of any one of SEQ ID NOS:1-8 and 82-97. The disclosure provides nucleic acids having at least 95% sequence identity to the nucleic acid sequence of any one of SEQ ID NOS:1-8 and 82-97. The disclosure provides nucleic acids having at least 96% sequence identity to the nucleic acid sequence of any one of SEQ ID NOS:1-8 and 82-97. The disclosure provides nucleic acids having at least 97% sequence identity to the nucleic acid sequence of any one of SEQ ID NOS:1-8 and 82-97. The disclosure provides nucleic acids having at least 98% sequence identity to the nucleic acid sequence of any one of SEQ ID NOS:1-8. The disclosure provides nucleic acids having at least 99% sequence identity to the nucleic acid sequence of any one of SEQ ID NOS:1-8 and 82-97. The disclosure provides nucleic acids having 100% sequence identity to the nucleic acid sequence of any one of SEQ ID NOS:1-8 and 82-97. In aspects, the nucleic acid comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid having the sequence identity described herein is SEQ ID NO:1. In aspects, the nucleic acid having the sequence identity described herein is SEQ ID NO:2. In aspects, the nucleic acid having the sequence identity described herein is SEQ ID NO:3. In aspects, the nucleic acid having the sequence identity described herein is SEQ ID NO:4. In aspects, the nucleic acid having the sequence identity described herein is SEQ ID NO:5. In aspects, the nucleic acid having the sequence identity described herein is SEQ ID NO:6. In aspects, the nucleic acid having the sequence identity described herein is SEQ ID NO:7. In aspects, the nucleic acid having the sequence identity described herein is SEQ ID NO: 8. In aspects, the nucleic acid having the sequence identity described herein is SEQ ID NO:82. In aspects, the nucleic acid having the sequence identity described herein is SEQ ID NO:83. In aspects, the nucleic acid having the sequence identity described herein is SEQ ID NO:84. In aspects, the nucleic acid having the sequence identity described herein is SEQ ID NO:85. In aspects, the nucleic acid having the sequence identity described herein is SEQ ID NO:86. In aspects, the nucleic acid having the sequence identity described herein is SEQ ID NO:87. In aspects, the nucleic acid having the sequence identity described herein is SEQ ID NO:88. In aspects, the nucleic acid having the sequence identity described herein is SEQ ID NO:89. In aspects, the nucleic acid having the sequence identity described herein is SEQ ID NO:90. In aspects, the nucleic acid having the sequence identity described herein is SEQ ID NO:91. In aspects, the nucleic acid having the sequence identity described herein is SEQ ID NO:92. In aspects, the nucleic acid having the sequence identity described herein is SEQ ID NO:93. In aspects, the nucleic acid having the sequence identity described herein is SEQ ID NO:94. In aspects, the nucleic acid having the sequence identity described herein is SEQ ID NO:95. In aspects, the nucleic acid having the sequence identity described herein is SEQ ID NO:96. In aspects, the nucleic acid is SEQ ID NO:97. In aspects, the nucleic acid is RNA. In aspects, the nucleic acid is antisense RNA. In aspects, the nucleic acid is siRNA.

The disclosure provides nucleic acids having at least 75% sequence identity to the nucleic acid sequence of any one of SEQ ID NOS:214-221 or 236-257. The disclosure provides nucleic acids having at least 80% sequence identity to the nucleic acid sequence of any one of SEQ ID NOS:214-221 or 236-257. The disclosure provides nucleic acids having at least 85% sequence identity to the nucleic acid sequence of any one of SEQ ID NOS:214-221 or 236-257. The disclosure provides nucleic acids having at least 90% sequence identity to the nucleic acid sequence of any one of SEQ ID NOS:214-221 or 236-257. The disclosure provides nucleic acids having at least 91% sequence identity to the nucleic acid sequence of any one of SEQ ID NOS:214-221 or 236-257. The disclosure provides nucleic acids having at least 92% sequence identity to the nucleic acid sequence of any one of SEQ ID NOS:214-221 or 236-257. The disclosure provides nucleic acids having at least 93% sequence identity to the nucleic acid sequence of any one of SEQ ID NOS:214-221 or 236-257. The disclosure provides nucleic acids having at least 94% sequence identity to the nucleic acid sequence of any one of SEQ ID NOS:214-221 or 236-257. The disclosure provides nucleic acids having at least 95% sequence identity to the nucleic acid sequence of any one of SEQ ID NOS:214-221 or 236-257. The disclosure provides nucleic acids having at least 96% sequence identity to the nucleic acid sequence of any one of SEQ ID NOS:214-221 or 236-257. The disclosure provides nucleic acids having at least 97% sequence identity to the nucleic acid sequence of any one of SEQ ID NOS:238-257. The disclosure provides nucleic acids having at least 98% sequence identity to the nucleic acid sequence of any one of SEQ ID NOS:214-221 or 236-257. The disclosure provides nucleic acids having at least 99% sequence identity to the nucleic acid sequence of any one of SEQ ID NOS:214-221 or 236-257. The disclosure provides nucleic acids having 100% sequence identity to the nucleic acid sequence of any one of SEQ ID NOS:214-221 or 236-257. In aspects, the nucleic acid comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid having the sequence identity described herein is SEQ ID NO:214. In aspects, the nucleic acid having the sequence identity described herein is SEQ ID NO:215. In aspects, the nucleic acid having the sequence identity described herein is SEQ ID NO:216. In aspects, the nucleic acid having the sequence identity described herein is SEQ ID NO:217. In aspects, the nucleic acid having the sequence identity described herein is SEQ ID NO:218. In aspects, the nucleic acid having the sequence identity described herein is SEQ ID NO:219. In aspects, the nucleic acid having the sequence identity described herein is SEQ ID NO:220. In aspects, the nucleic acid having the sequence identity described herein is SEQ ID NO:221. In aspects, the nucleic acid having the sequence identity described herein is SEQ ID NO:236. In aspects, the nucleic acid having the sequence identity described herein is SEQ ID NO:237. In aspects, the nucleic acid having the sequence identity described herein is SEQ ID NO:238. In aspects, the nucleic acid is SEQ ID NO:239. In aspects, the nucleic acid is SEQ ID NO:240. In aspects, the nucleic acid is SEQ ID NO:241. In aspects, the nucleic acid is SEQ ID NO:242. In aspects, the nucleic acid is SEQ ID NO:243. In aspects, the nucleic acid is SEQ ID NO:244. In aspects, the nucleic acid is SEQ ID NO:245. In aspects, the nucleic acid is SEQ ID NO:246. In aspects, the nucleic acid is SEQ ID NO:247. In aspects, the nucleic acid is SEQ ID NO:248. In aspects, the nucleic acid is SEQ ID NO:249. In aspects, the nucleic acid is SEQ ID NO:250. In aspects, the nucleic acid is SEQ ID NO:251. In aspects, the nucleic acid is SEQ ID NO:252. In aspects, the nucleic acid is SEQ ID NO:253. In aspects, the nucleic acid is SEQ ID NO:254. In aspects, the nucleic acid is SEQ ID NO:255. In aspects, the nucleic acid is SEQ ID NO:256. In aspects, the nucleic acid is SEQ ID NO:257. In aspects, the nucleic acid is RNA. In aspects, the nucleic acid is antisense RNA. In aspects, the nucleic acid is siRNA.

The disclosure provides nucleic acids having at least 75% sequence identity to a nucleic acid sequence in any one of Tables A-G. The disclosure provides nucleic acids having at least 80% sequence identity to a nucleic acid sequence in any one of Tables A-G. The disclosure provides nucleic acids having at least 85% sequence identity to a nucleic acid sequence in any one of Tables A-G. The disclosure provides nucleic acids having at least 90% sequence identity to a nucleic acid sequence in any one of Tables A-G. The disclosure provides nucleic acids having at least 91% sequence identity to a nucleic acid sequence in any one of Tables A-G. The disclosure provides nucleic acids having at least 92% sequence identity to a nucleic acid sequence in any one of Tables A-G. The disclosure provides nucleic acids having at least 93% sequence identity to a nucleic acid sequence in any one of Tables A-G. The disclosure provides nucleic acids having at least 94% sequence identity to a nucleic acid sequence in any one of Tables A-G. The disclosure provides nucleic acids having at least 95% sequence identity to a nucleic acid sequence in any one of Tables A-G. The disclosure provides nucleic acids having at least 96% sequence identity to a nucleic acid sequence in any one of Tables A-G. The disclosure provides nucleic acids having at least 97% sequence identity to a nucleic acid sequence in any one of Tables A-G. The disclosure provides nucleic acids having at least 98% sequence identity to a nucleic acid sequence in any one of Tables A-G. The disclosure provides nucleic acids having at least 99% sequence identity to a nucleic acid sequence in any one of Tables A-G. The disclosure provides nucleic acids having 100% sequence identity to a nucleic acid sequence in any one of Tables A-G. In aspects, the nucleic acid comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose.

In aspects, the nucleic acid having a sequence of any one of SEQ ID NOS:1-8 and 82-97 has: (i) at least one modified base, (ii) at least one modified sugar, (iii) at least one modified phosphate, or (iv) any combination of the foregoing. In aspects, the nucleic acid having a sequence of any one SEQ ID NOS:1-8 and 82-97 has from 2 to 15 modified bases. In aspects, the nucleic acid having a sequence of any one SEQ ID NOS:1-8 and 82-97 have from 4 to 12 modified bases. In aspects, the nucleic acid having a sequence of any one SEQ ID NOS:1-8 and 82-97 has from 5 to 10 modified bases. In aspects, the nucleic acid having a sequence of any one SEQ ID NOS:1-8 and 82-97 has from 6 to 10 modified bases. In aspects, the nucleic acid having a sequence of any one SEQ ID NOS:1-8 and 82-97 has from 7 to 9 modified bases. In aspects, the nucleic acid having a sequence of any one SEQ ID NOS:1-8 and 82-97 has from 0 to 5 modified phosphates. In aspects, the nucleic acid having a sequence of any one SEQ ID NOS:1-8 and 82-97 has from 0 to 3 modified phosphates. In aspects, the nucleic acid having a sequence of any one SEQ ID NOS:1-8 and 82-97 has from 1 to 5 modified phosphates. In aspects, the nucleic acid having a sequence of any one SEQ ID NOS:1-8 and 82-97 has from 1 to 3 modified phosphates. In aspects, the nucleic acid having a sequence of any one SEQ ID NOS:1-8 and 82-97 has from 1 or 2 modified phosphates. In aspects, the nucleic acid having a sequence of any one SEQ ID NOS:1-8 and 82-97 has from 0 to 5 modified sugars. In aspects, the nucleic acid having a sequence of any one SEQ ID NOS:1-8 and 82-97 has from 0 to 3 modified sugars. In aspects, the nucleic acid having a sequence of any one SEQ ID NOS:1-8 and 82-97 has from 0 to 2 modified sugars. In aspects, the nucleic acid having a sequence of any one SEQ ID NOS:1-8 and 82-97 has from 2 to 15 modified bases, from 0 to 5 modified phosphates, and from 0 to 5 modified sugars. In aspects, the nucleic acid having a sequence of any one SEQ ID NOS:1-8 and 82-97 has from 4 to 12 modified bases, from 0 to 3 modified phosphates, and from 0 to 3 modified sugars. In aspects, the nucleic acid having a sequence of any one SEQ ID NOS:1-8 and 82-97 has from 6 to 10 modified bases, from 1 to 3 modified phosphates, and from 0 to 2 modified sugars. In aspects, the modified base is a 2′O-Methyl modified base, a 2′O-methoxyethoxy modified base, a 2′fluoro modified base, a 5-methyl-cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is SEQ ID NO:1. In aspects, the nucleic acid is SEQ ID NO:3. In aspects, the nucleic acid is SEQ ID NO:5. In aspects, the nucleic acid is SEQ ID NO:7. In aspects, the nucleic acid is SEQ ID NO:82. In aspects, the nucleic acid is SEQ ID NO:83. In aspects, the nucleic acid is SEQ ID NO:84. In aspects, the nucleic acid is SEQ ID NO:85. In aspects, the nucleic acid is SEQ ID NO:86. In aspects, the nucleic acid is SEQ ID NO:87. In aspects, the nucleic acid is SEQ ID NO:88. In aspects, the nucleic acid is SEQ ID NO:89. In aspects, the nucleic acid is SEQ ID NO:90. In aspects, the nucleic acid is SEQ ID NO:91. In aspects, the nucleic acid is SEQ ID NO:92. In aspects, the nucleic acid is SEQ ID NO:93. In aspects, the nucleic acid is SEQ ID NO:94. In aspects, the nucleic acid is SEQ ID NO:95. In aspects, the nucleic acid is SEQ ID NO:96. In aspects, the nucleic acid is SEQ ID NO:97. In aspects, the nucleic acid is RNA. In aspects, the nucleic acid is antisense RNA. In aspects, the nucleic acid is siRNA.

In aspects, the nucleic acid having a sequence of any one of SEQ ID NOS:214-221 has: (i) at least one modified base, (ii) at least one modified sugar, (iii) at least one modified phosphate, or (iv) any combination of the foregoing. In aspects, the nucleic acid having a sequence of any one SEQ ID NOS:214-221 has from 2 to 15 modified bases. In aspects, the nucleic acid having a sequence of any one SEQ ID NOS:214-221 has from 4 to 12 modified bases. In aspects, the nucleic acid having a sequence of any one NOS:214-221 has from 5 to 10 modified bases. In aspects, the nucleic acid having a sequence of any one SEQ ID NOS:214-221 has from 6 to 10 modified bases. In aspects, the nucleic acid having a sequence of any one SEQ ID NOS:214-221 has from 7 to 9 modified bases. In aspects, the nucleic acid having a sequence of any one SEQ ID NOS:214-221 has from 0 to 5 modified phosphates. In aspects, the nucleic acid having a sequence of any one SEQ ID NOS:214-221 has from 0 to 3 modified phosphates. In aspects, the nucleic acid having a sequence of any one SEQ ID NOS:214-221 has from 1 to 5 modified phosphates. In aspects, the nucleic acid having a sequence of any one SEQ ID NOS:214-221 has from 1 to 3 modified phosphates. In aspects, the nucleic acid having a sequence of any one SEQ ID NOS:214-221 has from 1 or 2 modified phosphates. In aspects, the nucleic acid having a sequence of any one SEQ ID NOS:214-221 has from 0 to 5 modified sugars. In aspects, the nucleic acid having a sequence of any one SEQ ID NOS:214-221 has from 0 to 3 modified sugars. In aspects, the nucleic acid having a sequence of any one SEQ ID NOS:214-221 has from 0 to 2 modified sugars. In aspects, the nucleic acid having a sequence of any one SEQ ID NOS:214-221 has from 2 to 15 modified bases, from 0 to 5 modified phosphates, and from 0 to 5 modified sugars. In aspects, the nucleic acid having a sequence of any one SEQ ID NOS:214-221 has from 4 to 12 modified bases, from 0 to 3 modified phosphates, and from 0 to 3 modified sugars. In aspects, the nucleic acid having a sequence of any one SEQ ID NOS:214-221 has from 6 to 10 modified bases, from 1 to 3 modified phosphates, and from 0 to 2 modified sugars. In aspects, the modified base is a 2′O-Methyl modified base, a 2′O-methoxyethoxy modified base, a 2′fluoro modified base, a 5-methyl-cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is SEQ ID NO:214. In aspects, the nucleic acid is SEQ ID NO:215. In aspects, the nucleic acid is SEQ ID NO:216. In aspects, the nucleic acid is SEQ ID NO:217. In aspects, the nucleic acid is SEQ ID NO:218. In aspects, the nucleic acid is SEQ ID NO:219. In aspects, the nucleic acid is SEQ ID NO:220. In aspects, the nucleic acid is SEQ ID NO:221. In aspects, the nucleic acid is RNA. In aspects, the nucleic acid is antisense RNA. In aspects, the nucleic acid is siRNA.

In aspects, the nucleic acid having a sequence of any one of SEQ ID NOS:236-257 has: (i) at least one modified base, (ii) at least one modified sugar, (iii) at least one modified phosphate, or (iv) any combination of the foregoing. In aspects, the nucleic acid having a sequence of any one SEQ ID NOS:236-257 has from 2 to 15 modified bases. In aspects, the nucleic acid having a sequence of any one SEQ ID NOS:236-257 has from 4 to 12 modified bases. In aspects, the nucleic acid having a sequence of any one SEQ ID NOS:236-257 has from 5 to 10 modified bases. In aspects, the nucleic acid having a sequence of any one SEQ ID NOS:236-257 has from 6 to 10 modified bases. In aspects, the nucleic acid having a sequence of any one SEQ ID NOS:236-257 has from 7 to 9 modified bases. In aspects, the nucleic acid having a sequence of any one SEQ ID NOS:236-257 has from 0 to 5 modified phosphates. In aspects, the nucleic acid having a sequence of any one SEQ ID NOS:236-257 has from 0 to 3 modified phosphates. In aspects, the nucleic acid having a sequence of any one SEQ ID NOS:236-257 has from 1 to 5 modified phosphates. In aspects, the nucleic acid having a sequence of any one SEQ ID NOS:236-257 has from 1 to 3 modified phosphates. In aspects, the nucleic acid having a sequence of any one SEQ ID NOS:236-257 has from 1 or 2 modified phosphates. In aspects, the nucleic acid having a sequence of any one SEQ ID NOS:236-257 has from 0 to 5 modified sugars. In aspects, the nucleic acid having a sequence of any one SEQ ID NOS:236-257 has from 0 to 3 modified sugars. In aspects, the nucleic acid having a sequence of any one SEQ ID NOS:236-257 has from 0 to 2 modified sugars. In aspects, the nucleic acid having a sequence of any one SEQ ID NOS:236-257 has from 2 to 15 modified bases, from 0 to 5 modified phosphates, and from 0 to 5 modified sugars. In aspects, the nucleic acid having a sequence of any one SEQ ID NOS:236-257 has from 4 to 12 modified bases, from 0 to 3 modified phosphates, and from 0 to 3 modified sugars. In aspects, the nucleic acid having a sequence of any one SEQ ID NOS:236-257 has from 6 to 10 modified bases, from 1 to 3 modified phosphates, and from 0 to 2 modified sugars. In aspects, the modified base is a 2′O-Methyl modified base, a 2′O-methoxyethoxy modified base, a 2′fluoro modified base, a 5-methyl-cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is SEQ ID NO:236. In aspects, the nucleic acid is SEQ ID NO:237. In aspects, the nucleic acid is SEQ ID NO:238. In aspects, the nucleic acid is SEQ ID NO:239. In aspects, the nucleic acid is SEQ ID NO:240. In aspects, the nucleic acid is SEQ ID NO:241. In aspects, the nucleic acid is SEQ ID NO:242. In aspects, the nucleic acid is SEQ ID NO:243. In aspects, the nucleic acid is SEQ ID NO:244. In aspects, the nucleic acid is SEQ ID NO:245. In aspects, the nucleic acid is SEQ ID NO:246. In aspects, the nucleic acid is SEQ ID NO:247. In aspects, the nucleic acid is SEQ ID NO:248. In aspects, the nucleic acid is SEQ ID NO:249. In aspects, the nucleic acid is SEQ ID NO:250. In aspects, the nucleic acid is SEQ ID NO:251. In aspects, the nucleic acid is SEQ ID NO:252. In aspects, the nucleic acid is SEQ ID NO:253. In aspects, the nucleic acid is SEQ ID NO:254. In aspects, the nucleic acid is SEQ ID NO:255. In aspects, the nucleic acid is SEQ ID NO:256. In aspects, the nucleic acid is SEQ ID NO:257. In aspects, the nucleic acid is RNA. In aspects, the nucleic acid is antisense RNA. In aspects, the nucleic acid is siRNA.

In aspects, the nucleic acid having a sequence set forth in Tables A-G has: (i) at least one modified base, (ii) at least one modified sugar, (iii) at least one modified phosphate, or (iv) any combination of the foregoing. In aspects, the nucleic acid having a sequence set forth in Tables A-G has from 2 to 15 modified bases. In aspects, the nucleic acid having a sequence set forth in Tables A-G has from 4 to 12 modified bases. In aspects, the nucleic acid having a sequence set forth in Tables A-G has from 5 to 10 modified bases. In aspects, the nucleic acid having a sequence set forth in Tables A-G has from 6 to 10 modified bases. In aspects, the nucleic acid having a sequence set forth in Tables A-G has from 7 to 9 modified bases. In aspects, the nucleic acid having a sequence set forth in Tables A-G has from 0 to 5 modified phosphates. In aspects, the nucleic acid having a sequence set forth in Tables A-G has from 0 to 3 modified phosphates. In aspects, the nucleic acid having a sequence set forth in Tables A-G has from 1 to 5 modified phosphates. In aspects, the nucleic acid having a sequence set forth in Tables A-G has from 1 to 3 modified phosphates. In aspects, the nucleic acid having a sequence set forth in Tables A-G has from 1 or 2 modified phosphates. In aspects, the nucleic acid having a sequence set forth in Tables A-G has from 0 to 5 modified sugars. In aspects, the nucleic acid having a sequence set forth in Tables A-G has from 0 to 3 modified sugars. In aspects, the nucleic acid having a sequence set forth in Tables A-G has from 0 to 2 modified sugars. In aspects, the nucleic acid having a sequence set forth in Tables A-G has from 2 to 15 modified bases, from 0 to 5 modified phosphates, and from 0 to 5 modified sugars. In aspects, the nucleic acid having a sequence set forth in Tables A-G has from 4 to 12 modified bases, from 0 to 3 modified phosphates, and from 0 to 3 modified sugars. In aspects, the nucleic acid having a sequence set forth in Tables A-G has from 6 to 10 modified bases, from 1 to 3 modified phosphates, and from 0 to 2 modified sugars. In aspects, the modified base is a 2′O-Methyl modified base, a 2′O-methoxyethoxy modified base, a 2′fluoro modified base, a 5-methyl-cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose.

Tables A and B provided conserved siRNAs that target SARS-CoV-2 and SARS-CoV-1. Tables C and D provide ultra-conserved siRNAs targeted to SARS-CoV-2 with a single mismatch with MERS. Tables E and F provide siRNAs targeted to SARS-CoV-2. The nucleotides are ribose, except those marked by “(end quote) which are deoxyribose (e.g., A”). In embodiments of Tables A, B, C, D, E, F, and G, the disclosure provides the sense strand. In embodiments of Tables A, B, C, D, E, F, and G, the disclosure provides the antisense strand. In embodiments of Tables A, B, C, D, E, F, and G, the disclosure provides the sense strand hybridized to the corresponding antisense strand.

TABLE A

| Viral gene | | SiRNA | |
|---|---|---|---|
| target | Target CoV site 5′-3′ | strand | siRNA sequence (5′-3′) |
| Leader | CUCUUGUAGAUCUGUUCUCUA | Sense | CUUGUAGAUCUGUUCUCUAAA |
| Sequence | (SEQ ID NO: 100) | | (SEQ ID NO: 125) |
| (5′UTR) | | Antisense | UAGAGAACAGAUCUACAAGAG |
| | | | (SEQ ID NO: 126) |
| | UCUCUUGUAGAUCUGUUCUCU | Sense | UAGAGAACAGAUCUACAAGAG |
| | (SEQ ID NO: 101) | | (SEQ ID NO: 126) |
| | | Antisense | AGAGAACAGAUCUACAAGAGA |
| | | | (SEQ ID NO: 127) |
| 5′UTR/orf1a | GAAAGGUAAGAUGGAGAGCCU | Sense | AAGGUAAGAUGGAGAGCCUUG |
| | (SEQ ID NO: 102) | | (SEQ ID NO: 104) |
| | | Antisense | AGGCUCUCCAUCUUACCUUUC |
| | | | (SEQ ID NO: 128) |
| | AAAGGUAAGAUGGAGAGCCUU | Sense | AGGUAAGAUGGAGAGCCUUGU |
| | (SEQ ID NO: 103) | | (SEQ ID NO: 129) |
| | | Antisense | AAGGCUCUCCAUCUUACCUUU |
| | | | (SEQ ID NO: 130) |
| | AAGGUAAGAUGGAGAGCCUUG | Sense | GGUAAGAUGGAGAGCCUUGUC |
| | (SEQ ID NO: 104) | | (SEQ ID NO: 131) |
| | | Antisense | CAAGGCUCUCCAUCUUACCUU |
| | | | (SEQ ID NO: 132) |
| RdRp-slippery | UUAAACGGGUUUGCGGUGUAA | Sense | AAACGGGTTTGCGGTGTAAGT |
| site | (SEQ ID NO: 105) | | (SEQ ID NO: 133) |
| | | Antisense | UUACACCGCAAACCCGUUUAA |
| | | | (SEQ ID NO: 134) |
| RdRp | CAGGAUGUAAACUUACAUAGC | Sense | GGAUGUAAACUUACAUAGCUC |
| | (SEQ ID NO: 106) | | (SEQ ID NO: 135) |
| | | Antisense | GCUAUGUAAGUUUACAUCCUG |
| | | | (SEQ ID NO: 136) |
| | GUACAUAAUCAGGAUGUAAAC | Sense | ACAUAAUCAGGAUGUAAACUU |
| | (SEQ ID NO: 107) | | (SEQ ID NO: 137) |
| | | Antisense | GUUUACAUCCUGAUUAUGUAC |
| | | | (SEQ ID NO: 138) |
| 5′- | UUACAAACAAUUUGAUACUUA | Sense | ACAAACAAUUUGAUACUUAUA |
| 3′Exonuclease | (SEQ ID NO: 108) | | (SEQ ID NO: 139) |
| | | Antisense | UAAGUAUCAAAUUGUUUGUAA |
| | | | (SEQ ID NO: 140) |
| | UUUACAAACAAUUUGAUACUU | Sense | UACAAACAAUUUGAUACUUAU |
| | (SEQ ID NO: 109) | | (SEQ ID NO: 141) |
| | | Antisense | AAGUAUCAAAUUGUUUGUAAA |
| | | | (SEQ ID NO: 142) |
| Packaging | AUGGAGUCACAUUAAUUGGAG | Sense | GGAGUCACAUUAAUUGGAGAA |
| signal | (SEQ ID NO: 110) | | (SEQ ID NO: 143) |
| | | Antisense | CUCCAAUUAAUGUGACUCCAU |
| | | | (SEQ ID NO: 144) |
| | UGGAGUCACAUUAAUUGGAGA | Sense | GAGUCACAUUAAUUGGAGAAG |
| | (SEQ ID NO: 111) | | (SEQ ID NO: 145) |
| | | Antisense | UCUCCAAUUAAUGUGACUCCA |
| | | | (SEQ ID NO: 146) |
| Spike | UGGUUUUAAUUUUUCACAAAU | Sense | GUUUUAAUUUUUCACAAAUAU |
| | (SEQ ID NO: 112) | | (SEQ ID NO: 147) |
| | | Antisense | AUUUGUGAAAAAUUAAAACCA |
| | | | (SEQ ID NO: 148) |
| | GUGGUUUUAAUUUUUCACAAA | Sense | GGUUUUAAUUUUUCACAAAUA |
| | (SEQ ID NO: 113) | | (SEQ ID NO: 149) |
| | | Antisense | UUUGUGAAAAAUUAAAACCAC |
| | | | (SEQ ID NO: 150) |
| Membrane | GCUUGUUUUGUGCUUGCUGCU | Sense | UUGUUUUGUGCUUGCUGCUGU |
| | (SEQ ID NO: 114) | | (SEQ ID NO: 115) |
| | | Antisense | AGCAGCAAGCACAAAACAAGC |
| | | | (SEQ ID NO: 151) |
| | UUGUUUUGUGCUUGCUGCUGU | Sense | GUUUUGUGCUUGCUGCUGUUU |
| | (SEQ ID NO: 115) | | (SEQ ID NO: 152) |
| | | Antisense | ACAGCAGCAAGCACAAAACAA |
| | | | (SEQ ID NO: 153) |
| | AGUAAUAGGUUUCCUAUUCCU | Sense | UAAUAGGUUUCCUAUUCCUUA |
| | (SEQ ID NO: 116) | | (SEQ ID NO: 154) |
| | | Antisense | AGGAAUAGGAAACCUAUUACU |
| | | | (SEQ ID NO: 155) |
| | GUAAUAGGUUUCCUAUUCCUU | Sense | AAUAGGUUUCCUAUUCCUUAC |
| | (SEQ ID NO: 117) | | (SEQ ID NO: 156) |
| | | Antisense | AAGGAAUAGGAAACCUAUUAC |
| | | | (SEQ ID NO: 157) |
| 7a | AACGAACAUGAAAAUUAUUCU | Sense | CGAACAUGAAAAUUAUUCUUU |
| | (SEQ ID NO: 118) | | (SEQ ID NO: 158) |
| | | Antisense | AGAAUAAUUUUCAUGUUCGUU |
| | | | (SEQ ID NO: 159) |
| Nucleocapsid | AAGGCCAAACUGUCACUAAGA | Sense | GGCCAAACUGUCACUAAGAAA |
| | (SEQ ID NO: 119) | | (SEQ ID NO: 160) |
| | | Antisense | UCUUAGUGACAGUUUGGCCUU |
| | | | (SEQ ID NO: 161) |
| | GCCAAACUGUCACUAAGAAAU | Sense | CAAACUGUCACUAAGAAAUCU |
| | (SEQ ID NO: 120) | | (SEQ ID NO: 162) |
| | | Antisense | AUUUCUUAGUGACAGUUUGGC |
| | | | (SEQ ID NO: 163) |

TABLE B

| Viral gene | | SiRNA | |
|---|---|---|---|
| target | Target CoV site 5′-3′ | strand | siRNA sequence (5′-3′) |
| Leader | CUCUUGUAGAUCUGUUCUCUA | Sense | CUUGUAGAUCUGUUCUCUAAACGA″A″ |
| Sequence | (SEQ ID NO: 100) | | (SEQ ID NO: 164) |
| (5′UTR) | | Antisense | UUCGUUUAGAGAACAGAUCUACAAGAG |
| | | | (SEQ ID NO: 165) |
| | UCUCUUGUAGAUCUGUUCUCU | Sense | UCUUGUAGAUCUGUUCUCUAAACG″A″ |
| | (SEQ ID NO: 101) | | (SEQ ID NO: 166) |
| | | Antisense | UCGUUUAGAGAACAGAUCUACAAGAGA |
| | | | (SEQ ID NO: 167) |
| 5′UTR/orf1a | GAAAGGUAAGAUGGAGAGCCU | Sense | AAGGUAAGAUGGAGAGCCUUGUCC″C″ |
| | (SEQ ID NO: 102) | | (SEQ ID NO: 168) |
| | | Antisense | GGGACAAGGCUCUCCAUCUUACCUUUC |
| | | | (SEQ ID NO: 169) |
| | AAAGGUAAGAUGGAGAGCCUU | Sense | AGGUAAGAUGGAGAGCCUUGUCCC″T″ |
| | (SEQ ID NO: 103) | | (SEQ ID NO: 170) |
| | | Antisense | AGGGACAAGGCUCUCCAUCUUACCUUU |
| | | | (SEQ ID NO: 171) |
| | AAGGUAAGAUGGAGAGCCUUG | Sense | GGUAAGAUGGAGAGCCUUGUCCCT″G″ |
| | (SEQ ID NO: 104) | | (SEQ ID NO: 172) |
| | | Antisense | CAGGGACAAGGCUCUCCAUCUUACCUU |
| | | | (SEQ ID NO: 173) |
| RdRp- | UUAAACGGGUUUGCGGUGUAA | Sense | AAACGGGTTTGCGGTGTAAGTGCA″G″ |
| slippery site | (SEQ ID NO: 105) | | (SEQ ID NO: 174) |
| | | Antisense | CUGCACUUACACCGCAAACCCGUUUAA |
| | | | (SEQ ID NO: 175) |
| RdRp | CAGGAUGUAAACUUACAUAGC | Sense | GGAUGUAAACUUACAUAGCUCUAG″A″ |
| | (SEQ ID NO: 106) | | (SEQ ID NO: 176) |
| | | Antisense | UCUAGAGCUAUGUAAGUUUACAUCCUG |
| | | | (SEQ ID NO: 177) |
| | GUACAUAAUCAGGAUGUAAAC | Sense | ACAUAAUCAGGAUGUAAACUUACA″T″ |
| | (SEQ ID NO: 107) | | (SEQ ID NO: 178) |
| | | Antisense | AUGUAAGUUUACAUCCUGAUUAUGUAC |
| | | | (SEQ ID NO: 179) |
| 5′- | UUACAAACAAUUUGAUACUUA | Sense | ACAAACAAUUUGAUACUUAUAACC″T″ |
| 3′Exonuclease | (SEQ ID NO: 108) | | (SEQ ID NO: 180) |
| | | Antisense | AGGUUAUAAGUAUCAAAUUGUUUGUAA |
| | | | (SEQ ID NO: 181) |
| | UUUACAAACAAUUUGAUACUU | Sense | UACAAACAAUUUGAUACUUAUAAC″C″ |
| | (SEQ ID NO: 109) | | (SEQ ID NO: 182) |
| | | Antisense | GGUUAUAAGUAUCAAAUUGUUUGUAAA |
| | | | (SEQ ID NO: 183) |
| Packaging | AUGGAGUCACAUUAAUUGGAG | Sense | GGAGUCACAUUAAUUGGAGAAGCC″G″ |
| signal | (SEQ ID NO: 110) | | (SEQ ID NO: 184) |
| | | Antisense | CGGCUUCUCCAAUUAAUGUGACUCCAU |
| | | | (SEQ ID NO: 185) |
| | UGGAGUCACAUUAAUUGGAGA | Sense | G″AGUCACAUUAAUUGGAGAAGCCG″T″ |
| | (SEQ ID NO: 111) | | (SEQ ID NO: 186) |
| | | Antisense | ACGGCUUCUCCAAUUAAUGUGACUCCA |
| | | | (SEQ ID NO: 187) |
| Spike | UGGUUUUAAUUUUUCACAAAU | Sense | GUUUUAAUUUUUCACAAAUAUUAC″C″ |
| | (SEQ ID NO: 112) | | (SEQ ID NO: 188) |
| | | Antisense | GGUAAUAUUUGUGAAAAAUUAAAACCA |
| | | | (SEQ ID NO: 189) |
| | GUGGUUUUAAUUUUUCACAAA | Sense | GGUUUUAAUUUUUCACAAAUAUUA″C″ |
| | (SEQ ID NO: 113) | | (SEQ ID NO: 190) |
| | | Antisense | GUAAUAUUUGUGAAAAAUUAAAACCAC |
| | | | (SEQ ID NO: 191) |
| Membrane | GCUUGUUUUGUGCUUGCUGCU | Sense | UUGUUUUGUGCUUGCUGCUGUUUA″C″ |
| | (SEQ ID NO: 114) | | (SEQ ID NO: 192) |
| | | Antisense | GUAAACAGCAGCAAGCACAAAACAAGC |
| | | | (SEQ ID NO: 193) |
| | UUGUUUUGUGCUUGCUGCUGU | Sense | GUUUUGUGCUUGCUGCUGUUUACA″G″ |
| | (SEQ ID NO: 115) | | (SEQ ID NO: 194) |
| | | Antisense | CUGUAAACAGCAGCAAGCACAAAACAA |
| | | | (SEQ ID NO: 195) |
| | AGUAAUAGGUUUCCUAUUCCU | Sense | UAAUAGGUUUCCUAUUCCUUACAT″G″ |
| | (SEQ ID NO: 116) | | (SEQ ID NO: 196) |
| | | Antisense | CAUGUAAGGAAUAGGAAACCUAUUACU |
| | | | (SEQ ID NO: 197) |
| | GUAAUAGGUUUCCUAUUCCUU | Sense | AAUAGGUUUCCUAUUCCUUACAUG″G″ |
| | (SEQ ID NO: 117) | | (SEQ ID NO: 198) |
| | | Antisense | CCAUGUAAGGAAUAGGAAACCUAUUAC |
| | | | (SEQ ID NO: 199) |
| 7a | AACGAACAUGAAAAUUAUUCU | Sense | CGAACAUGAAAAUUAUUCUUUUCT″T″ |
| | (SEQ ID NO: 118) | | (SEQ ID NO: 200) |
| | | Antisense | AAGAAAAGAAUAAUUUUCAUGUUCGUU |
| | | | (SEQ ID NO: 201) |
| Nucleocapsid | AAGGCCAAACUGUCACUAAGA | Sense | GGCCAAACUGUCACUAAGAAAUCT″G″ |
| | (SEQ ID NO: 119) | | (SEQ ID NO: 202) |
| | | Antisense | CAGAUUUCUUAGUGACAGUUUGGCCUU |
| | | | (SEQ ID NO: 203) |
| | GCCAAACUGUCACUAAGAAAU | Sense | CAAACUGUCACUAAGAAAUCUGCT″G″ |
| | (SEQ ID NO: 120) | | (SEQ ID NO: 204) |
| | | Antisense | CAGCAGAUUUCUUAGUGACAGUUUGGC |
| | | | (SEQ ID NO: 205) |

TABLE C

| Viral gene | | SiRNA | |
|---|---|---|---|
| target | Target CoV site 5′-3′ | strand | siRNA sequence (5′-3′) |
| RdRp | CUUAUGGGUUGGGAUUAUCCU | Sense | UAUGGGUUGGGAUUAUCCUAA |
| | (SEQ ID NO: 121) | | (SEQ ID NO: 206) |
| | | Antisense | AGGAUAAUCCCAACCCAUAAG |
| | | | (SEQ ID NO: 207) |
| | UGCAAAGAAUAGAGCUCGCAC | Sense | CAAAGAAUAGAGCUCGCACCG |
| | (SEQ ID NO: 122) | | (SEQ ID NO: 208) |
| | | Antisense | GUGCGAGCUCUAUUCUUUGCA |
| | | | (SEQ ID NO: 209) |
| | ACUGUUGAUUCAUCACAGGGC | Sense | UGUUGAUUCAUCACAGGGCUC |
| | (SEQ ID NO: 123) | | (SEQ ID NO: 210) |
| | | Antisense | GCCCUGUGAUGAAUCAACAGU |
| | | | (SEQ ID NO: 211) |
| | UCACCUUAUAAUUCACAGAAU | Sense | ACCUUAUAAUUCACAGAAUGC |
| | (SEQ ID NO: 124) | | (SEQ ID NO: 212) |
| | | Antisense | AUUCUGUGAAUUAUAAGGUGA |
| | | | (SEQ ID NO: 213) |

TABLE D

| siRNA Code Name | siRNA Sequence (5′-3′) | Target CoV site 5′-3′ |
|---|---|---|
| SiRdRp1 | Sense: | CUUAUGGGUUGGGAUUAUCCU |
| | UAUGGGUUGGGAUUAUCCUAAAUG″T″ | (SEQ ID NO: 121) |
| | (SEQ ID NO: 214) | Gene Target: siRdRp |
| | Antisense: | |
| | ACAUUUAGGAUAAUCCCAACCCAUAAG | |
| | (SEQ ID NO: 215) | |
| siRdRp4 | Sense: | UGCAAAGAAUAGAGCUCGCAC |
| | CAAAGAAUAGAGCUCGCACCGUAG″C″ | (SEQ ID NO: 122) |
| | (SEQ ID NO: 216) | Gene Targe: siRdRp |
| | Antisense: | |
| | GCUACGGUGCGAGCUCUAUUCUUUGCA | |
| | (SEQ ID NO: 217) | |
| siHel1 | Sense: | ACUGUUGAUUCAUCACAGGGC |
| | UGUUGAUUCAUCACAGGGCUCAGA″A″ | (SEQ ID NO: 123) |
| | (SEQ ID NO: 218) | Gene Target: Helicase |
| | Antisense: | |
| | UUCUGAGCCCUGUGAUGAAUCAACAGU | |
| | (SEQ ID NO: 219) | |
| siHel2 | Sense: | UCACCUUAUAAUUCACAGAAU |
| | ACCUUAUAAUUCACAGAAUGCUGU″A″ | (SEQ ID NO: 124) |
| | (SEQ ID NO: 220) | Gene Target: Helicase |
| | Antisense: | |
| | UACAGCAUUCUGUGAAUUAUAAGGUGA | |
| | (SEQ ID NO: 221) | |
| RdRp1b | Sense: | CUUAUGGGUUGGGAUUACCCU |
| | UAUGGGUUGGGAUUACCCUAAAUG″T″ | (SEQ ID NO: 235) |
| | (SEQ ID NO: 236) | Gene Target: siRdRp |
| | Antisense: | |
| | ACAUUUAGGGUAAUCCCAACCCAUAAG | |
| | (SEQ ID NO: 237) | |

TABLE E

| Viral | | SiRNA | |
|---|---|---|---|
| Gene | | Code | |
| Target | Target SARS-COV-2 site 5′-3′ | Name | siRNA sequence (5′-3′) |
| 7a-E | CGAACUUAUGUACUCAUUCGU | siUC1 | Sense: |
| | (SEQ ID NO: 225) | | AACUUAUGUACUCAUUCGUUU |
| | | | (SEQ ID NO: 238) |
| | | | Antisense: |
| | | | ACGAAUGAGUACAUAAGUUCG |
| | | | (SEQ ID NO: 239) |
| 7a-E | GAACUUAUGUACUCAUUCGUU | siUC2 | Sense: |
| | (SEQ ID NO: 226) | | ACUUAUGUACUCAUUCGUUUC |
| | | | (SEQ ID NO: 240) |
| | | | Antisense: |
| | | | AACGAAUGAGUACAUAAGUUC |
| | | | (SEQ ID NO: 241) |
| Nsp8 | UCUUUGAAUGUGGCUAAAUCU | siUC3 | Sense: |
| | (SEQ ID NO: 227) | | UUUGAAUGUGGCUAAAUCUGA |
| | | | (SEQ ID NO: 242) |
| | | | Antisense: |
| | | | AGAUUUAGCCACAUUCAAAGA |
| | | | (SEQ ID NO: 243) |
| Nsp9 | GAACCACCUUGUAGGUUUGUU | siUC4 | Sense: |
| | (SEQ ID NO: 228) | | ACCACCUUGUAGGUUUGUUAC |
| | | | (SEQ ID NO: 244) |
| | | | Antisense: |
| | | | AACAAACCUACAAGGUGGUUC |
| | | | (SEQ ID NO: 245) |
| Nsp9 | ACUGGAACCACCUUGUAGGUU | siUC5 | Sense: |
| | (SEQ ID NO: 229) | | UGGAACCACCUUGUAGGUUUG |
| | | | (SEQ ID NO: 246) |
| | | | Antisense: |
| | | | AACCUACAAGGUGGUUCCAGU |
| | | | (SEQ ID NO: 247) |
| Nsp9 | GGAACCACCUUGUAGGUUUGU | siUC6 | Sense: |
| | (SEQ ID NO: 230) | | AACCACCUUGUAGGUUUGUUA |
| | | | (SEQ ID NO: 248) |
| | | | Antisense: |
| | | | ACAAACCUACAAGGUGGUUCC |
| | | | (SEQ ID NO: 249) |
| RdRp | GUUGGACUGAGACUGACCUUA | siUC7 | Sense: |
| | (SEQ ID NO: 231) | | UGGACUGAGACUGACCUUACU |
| | | | (SEQ ID NO: 250) |
| | | | Antisense: |
| | | | UAAGGUCAGUCUCAGUCCAAC |
| | | | (SEQ ID NO: 251) |
| Nsp9 | GAACUGGAACCACCUUGUAGG | siUC8 | Sense: |
| | (SEQ ID NO: 232) | | ACUGGAACCACCUUGUAGGUU |
| | | | (SEQ ID NO: 252) |
| | | | Antisense: |
| | | | CCUACAAGGUGGUUCCAGUUC |
| | | | (SEQ ID NO: 253) |
| RdRp | UGAUGAUGAUUAUUUCAAUAA | siUC9 | Sense: |
| | (SEQ ID NO: 233) | | AUGAUGAUUAUUUCAAUAAAA |
| | | | (SEQ ID NO: 254) |
| | | | Antisense: |
| | | | UUAUUGAAAUAAUCAUCAUCA |
| | | | (SEQ ID NO: 255) |
| RdRp | UCACUAUAUGUUAAACCAGGU | siUC10 | Sense: |
| | (SEQ ID NO: 234) | | ACUAUAUGUUAAACCAGGUGG |
| | | | (SEQ ID NO: 256) |
| | | | Antisense: |
| | | | ACCUGGUUUAACAUAUAGUGA |
| | | | (SEQ ID NO: 257) |

TABLE F

| SiRNA Code | | dsiRNA Sense (5′-3′) |
|---|---|---|
| Name | Target CoV-2 site 5′-3′ | siRNA Antisense (5′-3′) |
| SiUTR1 | UUGUCCCUGGUUUCAACGAGA | dsiRNA Sense (SEQ ID NO: 5): |
| | (SEQ ID NO: 223) | GUCCCUGGUUUCAACGAGAAAACA″C″ |
| | | siRNA Antisense (SEQ ID NO: 7): |
| | | GUGUUUUCUCGUUGAAACCAGGGACAA |
| SiUTR1 | SEQ ID NO: 223 | dsiRNA Sense: SEQ ID NO: 6 |
| Modified | | siRNA Antisense: SEQ ID NO: 8 |
| SiUTR2 | UGGCUUAGUAGAAGUUGAAAA | dsiRNA Sense (SEQ ID NO: 82): |
| | (SEQ ID NO: 258) | GCUUAGUAGAAGUUGAAAAAGGCG″T″ |
| | | siRNA Antisense (SEQ ID NO: 83): |
| | | ACGCCUUUUUCAACUUCUACUAAGCCA |
| SiUTR3 | UUAUACCUUCCCAGGUAACAA | dsiRNA Sense (SEQ ID NO: 1): |
| Unmodified | (SEQ ID NO: 224) | AUACCUUCCCAGGUAACAAACCAA″C″ |
| | | siRNA Antisense (SEQ ID NO: 3): |
| | | GUUGGUUUGUUACCUGGGAAGGUAUAA |
| SiUTR3 | SEQ ID NO: 224 | dsiRNA Sense: SEQ ID NO: 2 |
| Modified | | siRNA Antisense: SEQ ID NO: 4 |
| SiUTR4 | UAAAGAUGGCACUUGUGGCUU | dsiRNA Sense (SEQ ID NO: 84): |
| | (SEQ ID NO: 259) | AAGAUGGCACUUGUGGCUUAGUAG″A″ |
| | | siRNA Antisense (SEQ ID NO: 85): |
| | | UCUACUAAGCCACAAGUGCCAUCUUUA |
| SiUTR5 | GUCCCUGGUUUCAACGAGAAA | dsiRNA Sense (SEQ ID NO: 86): |
| | (SEQ ID NO: 260) | CCCUGGUUUCAACGAGAAAACACA″C″ |
| | | siRNA Antisense (SEQ ID NO: 87): |
| | | GUGUGUUUUCUCGUUGAAACCAGGGAC |

TABLE G

| SiRNA Code | | dsiRNA Sense (5′-3′) |
|---|---|---|
| Name | Target CoV-2 site 5′-3′ | siRNA Antisense (5′-3′) |
| siEnv1 | CUGUUUACAGAAUAAAUUGGA | dsiRNA Sense (SEQ ID NO: 88): |
| | (SEQ ID NO: 261) | GUUUACAGAAUAAAUUGGAUCACC″G″ |
| | | siRNA Antisense (SEQ ID NO: 89): |
| | | CGGUGAUCCAAUUUAUUCUGUAAACAG |
| siEnv2 | UGCUGUUUACAGAAUAAAUUG | dsiRNA Sense (SEQ ID NO: 90): |
| | (SEQ ID NO: 262) | CUGUUUACAGAAUAAAUUGGAUCA″C″ |
| | | siRNA Antisense (SEQ ID NO: 91): |
| | | GUGAUCCAAUUUAUUCUGUAAACAGCA |
| siEnv3 | UACUAUUACCGUUGAAGAGCU | dsiRNA Sense (SEQ ID NO: 92): |
| | (SEQ ID NO: 263) | CUAUUACCGUUGAAGAGCUUAAAA″A″ |
| | | siRNA Antisense (SEQ ID NO: 93): |
| | | UUUUUAAGCUCUUCAACGGUAAUAGUA |
| siEnv4 | CGGUACUAUUACCGUUGAAGA | dsiRNA Sense (SEQ ID NO: 94): |
| | (SEQ ID NO: 264) | GUACUAUUACCGUUGAAGAGCUUA″A″ |
| | | siRNA Antisense (SEQ ID NO: 95): |
| | | UUAAGCUCUUCAACGGUAAUAGUACCG |
| siEnv5 | CAGGAUUGGCAACUAUAAAUU | dsiRNA Sense (SEQ ID NO: 96): |
| | (SEQ ID NO: 265) | GGAUUGGCAACUAUAAAUUAAACA″C″ |
| | | siRNA Antisense (SEQ ID NO: 97): |
| | | GUGUUUAAUUUAUAGUUGCCAAUCCUG |

In embodiments, the disclosure provides asRNA sequences designed towards regions of the SARS-CoV-2 viral genome that are functionally important and have a high degree of sequence conservation between the SARS-CoV-1 and SARS-CoV-2 genomes, i.e., SEQ ID NOS:266-273. These regions include the SARS-CoV-2: (i) Nucleoprotein N genomic region that plays a critical role in virion assembly; (ii) 5′ region of the RNA-dependent RNA polymerase (nsp12) gene that includes the frameshift stimulating pseudoknot, inhibition of which is known to potently suppress viral replication, and; (iii) catalytic region of the RNA-dependent RNA polymerase (nsp12) gene, which is critical for the replication of the viral genome inside of the host cell. For each of these regions, the length of the was varied between 400 bp to 800 bp. As a control, an asRNA that is 400 bp in length was designed towards eGFP.

In embodiments, the disclosure provides a nucleic acid having at least 80% sequence identity to SEQ ID NO:266. In embodiments, the nucleic acid has at least 85% sequence identity to SEQ ID NO:266. In embodiments, the nucleic acid has at least 85% sequence identity to SEQ ID NO:266. In embodiments, the nucleic acid has at least 90% sequence identity to SEQ ID NO:266. In embodiments, the nucleic acid has at least 92% sequence identity to SEQ ID NO:266. In embodiments, the nucleic acid has at least 94% sequence identity to SEQ ID NO:266. In embodiments, the nucleic acid has at least 95% sequence identity to SEQ ID NO:266. In embodiments, the nucleic acid has at least 96% sequence identity to SEQ ID NO:266. In embodiments, the nucleic acid has at least 98% sequence identity to SEQ ID NO:266. In embodiments, the nucleic acid has SEQ ID NO:266. In embodiments, the nucleic acid comprises SEQ ID NO:266. In aspects, the nucleic acid of SEQ ID NO:266 comprises: (i) at least one modified base, (ii) at least one modified sugar, (iii) at least one modified phosphate, or (iv) any combination of the foregoing. In aspects, the nucleic acid of SEQ ID NO:266 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In embodiments, the disclosure provides a nucleic acid having at least 80% sequence identity to SEQ ID NO:267. In embodiments, the nucleic acid has at least 85% sequence identity to SEQ ID NO:267. In embodiments, the nucleic acid has at least 85% sequence identity to SEQ ID NO:267. In embodiments, the nucleic acid has at least 90% sequence identity to SEQ ID NO:267. In embodiments, the nucleic acid has at least 92% sequence identity to SEQ ID NO:267. In embodiments, the nucleic acid has at least 94% sequence identity to SEQ ID NO:267. In embodiments, the nucleic acid has at least 95% sequence identity to SEQ ID NO:267. In embodiments, the nucleic acid has at least 96% sequence identity to SEQ ID NO:267. In embodiments, the nucleic acid has at least 98% sequence identity to SEQ ID NO:267. In embodiments, the nucleic acid has SEQ ID NO:267. In embodiments, the nucleic acid comprises SEQ ID NO:267. In aspects, the nucleic acid of SEQ ID NO:267 comprises: (i) at least one modified base, (ii) at least one modified sugar, (iii) at least one modified phosphate, or (iv) any combination of the foregoing. In aspects, the nucleic acid of SEQ ID NO:267 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In embodiments, the disclosure provides a nucleic acid having at least 80% sequence identity to SEQ ID NO:268. In embodiments, the nucleic acid has at least 85% sequence identity to SEQ ID NO:268. In embodiments, the nucleic acid has at least 85% sequence identity to SEQ ID NO:268. In embodiments, the nucleic acid has at least 90% sequence identity to SEQ ID NO:268. In embodiments, the nucleic acid has at least 92% sequence identity to SEQ ID NO:268. In embodiments, the nucleic acid has at least 94% sequence identity to SEQ ID NO:268. In embodiments, the nucleic acid has at least 95% sequence identity to SEQ ID NO:268. In embodiments, the nucleic acid has at least 96% sequence identity to SEQ ID NO:268. In embodiments, the nucleic acid has at least 98% sequence identity to SEQ ID NO:268. In embodiments, the nucleic acid has SEQ ID NO:268. In embodiments, the nucleic acid comprises SEQ ID NO:268. In aspects, the nucleic acid of SEQ ID NO:268 comprises: (i) at least one modified base, (ii) at least one modified sugar, (iii) at least one modified phosphate, or (iv) any combination of the foregoing. In aspects, the nucleic acid of SEQ ID NO:268 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In embodiments, the disclosure provides a nucleic acid having at least 80% sequence identity to SEQ ID NO:269. In embodiments, the nucleic acid has at least 85% sequence identity to SEQ ID NO:269. In embodiments, the nucleic acid has at least 85% sequence identity to SEQ ID NO:269. In embodiments, the nucleic acid has at least 90% sequence identity to SEQ ID NO:269. In embodiments, the nucleic acid has at least 92% sequence identity to SEQ ID NO:269. In embodiments, the nucleic acid has at least 94% sequence identity to SEQ ID NO:269. In embodiments, the nucleic acid has at least 95% sequence identity to SEQ ID NO:269. In embodiments, the nucleic acid has at least 96% sequence identity to SEQ ID NO:269. In embodiments, the nucleic acid has at least 98% sequence identity to SEQ ID NO:269. In embodiments, the nucleic acid has SEQ ID NO:269. In embodiments, the nucleic acid comprises SEQ ID NO:269. In aspects, the nucleic acid of SEQ ID NO:269 comprises: (i) at least one modified base, (ii) at least one modified sugar, (iii) at least one modified phosphate, or (iv) any combination of the foregoing. In aspects, the nucleic acid of SEQ ID NO:269 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In embodiments, the disclosure provides a nucleic acid having at least 80% sequence identity to SEQ ID NO:270. In embodiments, the nucleic acid has at least 85% sequence identity to SEQ ID NO:270. In embodiments, the nucleic acid has at least 85% sequence identity to SEQ ID NO:270. In embodiments, the nucleic acid has at least 90% sequence identity to SEQ ID NO:270. In embodiments, the nucleic acid has at least 92% sequence identity to SEQ ID NO:270. In embodiments, the nucleic acid has at least 94% sequence identity to SEQ ID NO:270. In embodiments, the nucleic acid has at least 95% sequence identity to SEQ ID NO:270. In embodiments, the nucleic acid has at least 96% sequence identity to SEQ ID NO:270. In embodiments, the nucleic acid has at least 98% sequence identity to SEQ ID NO:270. In embodiments, the nucleic acid has SEQ ID NO:270. In embodiments, the nucleic acid comprises SEQ ID NO:270. In aspects, the nucleic acid of SEQ ID NO:270 comprises: (i) at least one modified base, (ii) at least one modified sugar, (iii) at least one modified phosphate, or (iv) any combination of the foregoing. In aspects, the nucleic acid of SEQ ID NO:270 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In embodiments, the disclosure provides a nucleic acid having at least 80% sequence identity to SEQ ID NO:271. In embodiments, the nucleic acid has at least 85% sequence identity to SEQ ID NO:271. In embodiments, the nucleic acid has at least 85% sequence identity to SEQ ID NO:271. In embodiments, the nucleic acid has at least 90% sequence identity to SEQ ID NO:271. In embodiments, the nucleic acid has at least 92% sequence identity to SEQ ID NO:271. In embodiments, the nucleic acid has at least 94% sequence identity to SEQ ID NO:271. In embodiments, the nucleic acid has at least 95% sequence identity to SEQ ID NO:271. In embodiments, the nucleic acid has at least 96% sequence identity to SEQ ID NO:271. In embodiments, the nucleic acid has at least 98% sequence identity to SEQ ID NO:271. In embodiments, the nucleic acid has SEQ ID NO:271. In embodiments, the nucleic acid comprises SEQ ID NO:271. In aspects, the nucleic acid of SEQ ID NO:271 comprises: (i) at least one modified base, (ii) at least one modified sugar, (iii) at least one modified phosphate, or (iv) any combination of the foregoing. In aspects, the nucleic acid of SEQ ID NO:271 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In embodiments, the disclosure provides a nucleic acid having at least 80% sequence identity to SEQ ID NO:272. In embodiments, the nucleic acid has at least 85% sequence identity to SEQ ID NO:272. In embodiments, the nucleic acid has at least 85% sequence identity to SEQ ID NO:272. In embodiments, the nucleic acid has at least 90% sequence identity to SEQ ID NO:272. In embodiments, the nucleic acid has at least 92% sequence identity to SEQ ID NO:272. In embodiments, the nucleic acid has at least 94% sequence identity to SEQ ID NO:272. In embodiments, the nucleic acid has at least 95% sequence identity to SEQ ID NO:272. In embodiments, the nucleic acid has at least 96% sequence identity to SEQ ID NO:272. In embodiments, the nucleic acid has at least 98% sequence identity to SEQ ID NO:272. In embodiments, the nucleic acid has SEQ ID NO:272. In embodiments, the nucleic acid comprises SEQ ID NO:272. In aspects, the nucleic acid of SEQ ID NO:272 comprises: (i) at least one modified base, (ii) at least one modified sugar, (iii) at least one modified phosphate, or (iv) any combination of the foregoing. In aspects, the nucleic acid of SEQ ID NO:272 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

In embodiments, the disclosure provides a nucleic acid having at least 80% sequence identity to SEQ ID NO:273. In embodiments, the nucleic acid has at least 85% sequence identity to SEQ ID NO:273. In embodiments, the nucleic acid has at least 85% sequence identity to SEQ ID NO:273. In embodiments, the nucleic acid has at least 90% sequence identity to SEQ ID NO:273. In embodiments, the nucleic acid has at least 92% sequence identity to SEQ ID NO:273. In embodiments, the nucleic acid has at least 94% sequence identity to SEQ ID NO:273. In embodiments, the nucleic acid has at least 95% sequence identity to SEQ ID NO:273. In embodiments, the nucleic acid has at least 96% sequence identity to SEQ ID NO:273. In embodiments, the nucleic acid has at least 98% sequence identity to SEQ ID NO:273. In embodiments, the nucleic acid has SEQ ID NO:273. In embodiments, the nucleic acid comprises SEQ ID NO:273. In aspects, the nucleic acid of SEQ ID NO:273 comprises: (i) at least one modified base, (ii) at least one modified sugar, (iii) at least one modified phosphate, or (iv) any combination of the foregoing. In aspects, the nucleic acid of SEQ ID NO:273 comprises: (i) at least one modified base, (ii) at least one modified sugar, (iii) at least one modified phosphate, or (iv) any combination of the foregoing. In aspects, the modified base is a 2′O-Methyl modified base, a 2′O-methoxyethoxy modified base, a 2′fluoro modified base, a 5′-methyl-modified cytidine, or pseudouridine. In aspects, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In aspects, the modified base is a 2′O-Methyl modified base. In aspects, the modified phosphate is phosphorothioate. In aspects, the modified sugar is deoxyribose. In aspects, the nucleic acid is RNA. In aspects, the nucleic acid is antisense RNA. In aspects, the nucleic acid is siRNA.

Lipid Nanoparticles

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats, oils, and waxes; (2) “compound lipids,” which include phospholipids and glycolipids; and (3) “derived lipids” such as steroids.

The terms “lipid particle” and “lipid nanoparticle” and “stealth lipid nanoparticle” refer to a lipid formulation that can be used to deliver an active agent or therapeutic agent, such as a nucleic acid (e.g., RNA, sense RNA, antisense RNA, siRNA), to a target site of interest (e.g., cell, tissue, organ, and the like). In aspects, the lipid particle is a nucleic acid-lipid particle, which is typically formed from a cationic lipid, a non-cationic lipid, and optionally a conjugated lipid that prevents aggregation of the particle. In other aspects, the active agent or therapeutic agent, such as a nucleic acid (e.g., RNA, sense RNA, antisense RNA, siRNA), may be encapsulated in the lipid portion of the particle, thereby protecting it from enzymatic degradation.

The term “SNALP” refers to a stable nucleic acid-lipid particle. A SNALP represents a particle made from lipids (e.g., a cationic lipid, a non-cationic lipid, and optionally a conjugated lipid that prevents aggregation of the particle), wherein the nucleic acid (e.g., RNA, sense RNA, antisense RNA, siRNA) is fully encapsulated within the lipid. In aspects, SNALP are useful for systemic applications, as they can exhibit extended circulation lifetimes following intravenous injection, they can accumulate at distal sites (e.g., sites physically separated from the administration site), and they can mediate silencing of target gene expression at these distal sites. The nucleic acid (e.g., RNA, sense RNA, antisense RNA, siRNA) may be complexed with a condensing agent and encapsulated within a SNALP as set forth, e.g., in WO 2000/03683.

The lipid particles typically have a mean diameter of from about 10 nm to about 200 nm, from about 20 nm to about 190 nm, from about 30 nm to about 175 nm, from about 40 nm to about 160 nm, from about 50 nm to about 150 nm, from about 60 nm to about 140 nm, from about 90 nm to about 130 nm, from about 100 nm to about 130 nm. In aspects the lipid particles have a mean diameter of about 125 nm. In aspects, the lipid particles have a mean diameter from about 120 nm to about 130 nm. In aspects, the lipid particles have a mean diameter from about 110 nm to about 140 nm. In aspects, the lipid particles have a mean diameter from about 100 nm to about 150 nm. In addition, nucleic acids (e.g., RNA, sense RNA, antisense RNA, siRNA), when present in the lipid particles, are resistant in aqueous solution to degradation with a nuclease. Nucleic acid-lipid particles and their method of preparation are disclosed, e.g., in US Publication Nos. 2004/0142025 and 2007/0042031.

“Lipid encapsulated” can refer to a lipid particle that provides an active agent or therapeutic agent, such as a nucleic acid (e.g., RNA, sense RNA, antisense RNA, siRNA), with full encapsulation, partial encapsulation, or both. In aspects, the nucleic acid (e.g., RNA, sense RNA, antisense RNA, siRNA) is fully encapsulated in the lipid particle (e.g., to form a SNALP or other nucleic acid-lipid particle). In embodiments, “lipid encapsulated” refers to a lipid particle that contains two to six different active agents or therapeutic agents, such as a nucleic acid (e.g., RNA, sense RNA, antisense RNA, siRNA), with full encapsulation, partial encapsulation, or both. In embodiments, “lipid encapsulated” refers to a lipid particle that contains two to five different active agents or therapeutic agents, such as a nucleic acid (e.g., RNA, sense RNA, antisense RNA, siRNA), with full encapsulation, partial encapsulation, or both. In embodiments, “lipid encapsulated” refers to a lipid particle that contains two to four different active agents or therapeutic agents, such as a nucleic acid (e.g., RNA, sense RNA, antisense RNA, siRNA), with full encapsulation, partial encapsulation, or both. In embodiments, “lipid encapsulated” refers to a lipid particle that contains two or three different active agents or therapeutic agents, such as a nucleic acid (e.g., RNA, sense RNA, antisense RNA, siRNA), with full encapsulation, partial encapsulation, or both. In embodiments, “lipid encapsulated” refers to a lipid particle that contains two different active agents or therapeutic agents, such as a nucleic acid (e.g., RNA, sense RNA, antisense RNA, siRNA), with full encapsulation, partial encapsulation, or both. In embodiments, “lipid encapsulated” refers to a lipid particle that contains three different active agents or therapeutic agents, such as a nucleic acid (e.g., RNA, sense RNA, antisense RNA, siRNA), with full encapsulation, partial encapsulation, or both. In embodiments, “lipid encapsulated” refers to a lipid particle that contains four different active agents or therapeutic agents, such as a nucleic acid (e.g., RNA, sense RNA, antisense RNA, siRNA), with full encapsulation, partial encapsulation, or both.

The term “lipid conjugate” refers to a conjugated lipid that inhibits aggregation of lipid particles. Such lipid conjugates include PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., US Publication No. 2011/0313017), polyamide oligomers (e.g., ATTA-lipid conjugates), and mixtures thereof. Additional examples of POZ-lipid conjugates are described in WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In aspects, non-ester containing linker moieties, such as amides or carbamates, are used.

The term “amphipathic lipid” refers, in part, to any material wherein the hydrophobic portion of the lipid orients into a hydrophobic phase, while the hydrophilic portion orients toward the aqueous phase. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include long-chain saturated and unsaturated aliphatic hydrocarbon groups and such groups substituted by one or more aromatic, cycloaliphatic, or heterocyclic group(s). Examples of amphipathic compounds include phospholipids, aminolipids, and sphingolipids. Representative examples of phospholipids include phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and O-acyloxyacids, are also within the group designated as amphipathic lipids. Additionally, the amphipathic lipids can be mixed with other lipids including triglycerides and sterols.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “hydrophobic lipid” refers to compounds having apolar groups that include long-chain saturated and unsaturated aliphatic hydrocarbon groups and such groups optionally substituted by one or more aromatic, cycloaliphatic, or heterocyclic group(s). Suitable examples include diacylglycerol, dialkylglycerol, N—N-dialkylamino, 1,2-diacyloxy-3-aminopropane, and 1,2-dialkyl-3-aminopropane.

The term “non-lamellar morphology” refer to a non-bilayer structure. The non-bilayer morphology can include, for example, three dimensional tubes, rods, cubic symmetries, etc. The non-lamellar morphology (i.e., non-bilayer structure) of the lipid particles can be determined using analytical techniques including Cryo-Transmission Electron Microscopy (“Cryo-TEM”), Differential Scanning calorimetry (“DSC”), and X-Ray Diffraction.

The term “a plurality of nucleic acid-lipid particles” refers to at least 2 particles, more preferably more than 10, 102, 103, 104, 105, 106 or more particles (or any fraction thereof or range therein). In aspects, the plurality of nucleic acid-lipid particles includes 50-100, 50-200, 50-300, 50-400, 50-500, 50-600, 50-700, 50-800, 50-900, 50-1000, 50-1100, 50-1200, 50-1300, 50-1400, 50-1500, 50-1600, 50-1700, 50-1800, 50-1900, 50-2000, 50-2500, 50-3000, 50-3500, 50-4000, 50-4500, 50-5000, 50-5500, 50-6000, 50-6500, 50-7000, 50-7500, 50-8000, 50-8500, 50-9000, 50-9500, 50-10,000 or more particles.

The term “organic lipid solution” refers to a composition comprising in whole, or in part, an organic solvent having a lipid.

In embodiments, the disclosure provides lipid nanoparticles comprising the nucleic acids (e.g., RNA, sense RNA, sense RNA, antisense RNA, siRNA) described herein. Exemplary lipid nanoparticles and methods for making them are described in US Publication No. 2017/0143631, US Publication No. 2018/0043009, US Publication No. 2018/0064807, US Publication No. 2018/0065918, US Publication No. 2018/0092971, US Publication No. 2018/0125985, US Publication No. 2018/0104342, US Publication No. 2018/0221510, US Publication No. 2018/0369384, US Publication No. 2019/0167800, US Publication No. 2019/0106379, U.S. Pat. Nos. 7,982,027, 9,005,654, 9,352,042, 9,364,435, 9,394,234, 9,404,127, 9,518,272, 9,663,449, 9,682,139, 9,687,550, 9,694,077, 9,707,292, 9,764,036 U.S. Pat. Nos. 9,814,777, 10,077,232, 10,117,941, 10,456,473, 10,561,732, and Villamizar et al, Molecular Therapy, 27(10) (October 2019), the disclosures of which are incorporated by reference herein in their entirety.

In aspects, the lipid nanoparticle comprises a nucleic acid (e.g., RNA, sense RNA, antisense RNA, siRNA) described herein and a lipid. In aspects, the lipid nanoparticle comprises two to six different nucleic acids (e.g., RNA, sense RNA, sense RNA, antisense RNA, siRNA) described herein and a lipid. In aspects, the lipid nanoparticle comprises two to five different nucleic acids (e.g., RNA, sense RNA, sense RNA, antisense RNA, siRNA) described herein and a lipid. In aspects, the lipid nanoparticle comprises two to four different nucleic acids (e.g., RNA, sense RNA, sense RNA, antisense RNA, siRNA) described herein and a lipid. In aspects, the lipid nanoparticle comprises two or three different nucleic acids (e.g., RNA, sense RNA, antisense RNA, siRNA) described herein and a lipid. In aspects, the lipid nanoparticle comprises two different nucleic acids (e.g., RNA, sense RNA, sense RNA, antisense RNA, siRNA) described herein and a lipid. In aspects, the lipid nanoparticle comprises three different nucleic acids (e.g., RNA, sense RNA, sense RNA, antisense RNA, siRNA) described herein and a lipid. In aspects, the lipid nanoparticle comprises four different nucleic acid (e.g., RNA, sense RNA, sense RNA, antisense RNA, siRNA) described herein and a lipid. In aspects, the lipid is a cationic lipid, a neutral lipid, an anionic lipid, a non-cationic lipid, a conjugated lipid, or a combination of two or more thereof. In aspects, the term non-cationic lipid refers to a neutral lipid, an anionic lipid, or a combination thereof.

The cationic lipid may be any known in the art. Exemplary cationic lipids include MC3, LenMC3, CP-LenMC3, γ-LenMC3, CP-γ-LenMC3, MC3MC, MC2MC, MC3 Ether, MC4 Ether, MC3 Amide, Pan-MC3, Pan-MC4 and Pan MC5, 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyOcholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethy-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), or mixtures thereof. In certain preferred embodiments, the cationic lipid is DLinDMA, DLin-K-C2-DMA (“XTC2”), MC3, LenMC3, CP-LenMC3, γ-LenMC3, CP-′γ-LenMC3, MC3MC, MC2MC, MC3 Ether, MC4 Ether, MC3 Amide, Pan-MC3, Pan-MC4, Pan MC5, or mixtures thereof.

In aspects, any non-cationic lipid known in the art can be used. Exemplary non-cationic lipids include anionic lipids, neutral lipids, or a combination thereof. In aspects, the non-cationic lipid comprises one of the following neutral lipid components: (1) cholesterol or a derivative thereof; (2) a phospholipid; or (3) a mixture of a phospholipid and cholesterol or a derivative thereof.

Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof.

The phospholipid may be a neutral lipid including dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof.

In the lipid particles described herein, the conjugated lipid that inhibits aggregation of particles may comprise one or more of the following: a polyethyleneglycol (PEG)-lipid conjugate, a polyamide (ATTA)-lipid conjugate, a cationic-polymer-lipid conjugates (CPLs), or a mixture of two or more thereof. In aspects, the nucleic acid-lipid particles comprise either a PEG-lipid conjugate or an ATTA-lipid conjugate. In aspects, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or a mixture of two or more thereof. The PEG-DAA conjugate may be PEG-dilauryloxypropyl (C12), a PEG-dimyristyloxypropyl (C14), a PEG-dipalmityloxypropyl (C16), a PEG-distearyloxypropyl (C18), or a mixture of two or more thereof.

In aspects, the lipid nanoparticles comprise a nucleic acid (e.g., RNA, sense RNA, sense RNA, antisense RNA, siRNA) described herein and one or more lipids. In aspects, the lipids comprise DOTAP, cholesterol, DOPE, PEG conjugated to ceramide, or a combination of two or more thereof. In aspects, the PEG conjugated to ceramide is polyethylene glycol having a molecular weight of about 2000 conjugated to C₁₆ceramide. In aspects, the molar ration of DOTAP to cholesterol to DOPE to PEG-ceramide is 40-60:30-40:1-10:5-15. In aspects, the molar ration of DOTAP to cholesterol to DOPE to PEG-ceramide is 50:35:5:10. Such formulations are described, for example, by McCaskill et al, Molecular Therapy-Nucleic Acids (2013) 2(6):e96; doi:10.1038/mtna.2013.22; and Wu et al, Pharmaceutical Research, 26(3):512-522 (2008).

In embodiments, the pharmaceutical compositions comprising the lipid nanoparticles comprise an isotonic solution. An exemplary isotonic solution is an isotonic sucrose solution.

Pharmaceutical Compositions

Provided herein are pharmaceutical compositions comprising nucleic acids (e.g., RNA, sense RNA, sense RNA, antisense RNA, siRNA, lipid nanoparticles containing nucleic acids) and a pharmaceutically acceptable excipient. In embodiments, the pharmaceutical composition comprises nucleic acids (e.g., RNA, sense RNA, sense RNA, antisense RNA, siRNA) and a pharmaceutically acceptable excipient. In embodiments, the pharmaceutical composition comprises lipid nanoparticles and a pharmaceutically acceptable excipient; wherein the lipid nanoparticles comprise nucleic acids (e.g., RNA, sense RNA, sense RNA, antisense RNA, siRNA).

In embodiments, the pharmaceutical compositions comprise a first lipid nanoparticle which comprises a first nucleic acid (e.g., RNA, sense RNA, sense RNA, antisense RNA, siRNA) and a second lipid nanoparticle which comprises a second nucleic acid (e.g., RNA, sense RNA, sense RNA, antisense RNA, siRNA); wherein the first lipid nanoparticle and the second lipid nanoparticle are the same or different, and wherein the first nucleic acid and second nucleic acid are different. In embodiments, the first lipid nanoparticle and the second lipid nanoparticle are the same (i.e., comprise the same lipids). In embodiments, the pharmaceutical compositions comprise a first lipid nanoparticle which comprises a first nucleic acid (e.g., RNA, sense RNA, sense RNA, antisense RNA, siRNA); a second lipid nanoparticle which comprises a second nucleic acid (e.g., RNA, sense RNA, sense RNA, antisense RNA, siRNA); and a third lipid nanoparticle which comprises a third nucleic acid (e.g., RNA, sense RNA, sense RNA, antisense RNA, siRNA); wherein the first, second, and third lipid nanoparticles are the same or different, and wherein the first, second, and third nucleic acids are different. In embodiments, the first, second, and third lipid nanoparticles are the same (i.e., comprise the same lipids). In embodiments, the first, second, and third nucleic acids are selected from the group consisting of siUTR3, siUC7, siHel2, siUTR1, siUC1, siUC6, and siHel1, and the lipid nanoparticles comprise the same lipid components. In embodiments, the first, second, and third nucleic acids are siUTR3, siUC7, and siHel2, and the lipid nanoparticles comprise the same lipid components. In embodiments, the pharmaceutical compositions comprise a first lipid nanoparticle which comprises a first nucleic acid (e.g., RNA, sense RNA, sense RNA, antisense RNA, siRNA); a second lipid nanoparticle which comprises a second nucleic acid (e.g., RNA, sense RNA, sense RNA, antisense RNA, siRNA); a third lipid nanoparticle which comprises a third nucleic acid (e.g., RNA, sense RNA, sense RNA, antisense RNA, siRNA); a fourth lipid nanoparticle which comprises a fourth nucleic acid (e.g., RNA, sense RNA, sense RNA, antisense RNA, siRNA); wherein the first, second, third, and fourth lipid nanoparticles are the same or different, and wherein the first, second, third, and fourth nucleic acids are different. In embodiments, the first, second, third, and fourth lipid nanoparticles are the same (i.e., comprise the same lipids). The pharmaceutical compositions can optionally further comprises a fifth lipid nanoparticle which comprises a fifth nucleic acid (e.g., RNA, sense RNA, sense RNA, antisense RNA, siRNA); wherein the first, second, third, fourth, and fifth lipid nanoparticles are the same or different, and wherein the first, second, third, fourth, and fifth nucleic acids are different. In embodiments, the first, second, third, fourth, and fifth lipid nanoparticles are the same (i.e., comprise the same lipids). The compositions are suitable for formulation and administration in vitro or in vivo. Suitable carriers and excipients and their formulations are known in the art and described, e.g., in Remington: The Science and Practice of Pharmacy, 21st Edition, David B. Troy, ed., Lippicott Williams & Wilkins (2005).

“Pharmaceutically acceptable excipient” and “pharmaceutically acceptable carrier” refer to a substance that aids the administration of an active agent to and absorption by a subject and can be included in the compositions of the disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions, alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful.

Solutions of the nucleic acids or lipid nanoparticles containing nucleic acids can be prepared in water suitably mixed with a lipid or surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions. The intranasal composition can be a spray, aerosol, or inhalant. The inhalable composition can be a spray, aerosol, or inhalant. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known in the art.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In aspects, oral pharmaceutical compositions will comprise an inert diluent or edible carrier, or they may be enclosed in hard or soft shell gelatin capsule, or they may be compressed into tablets, or they may be incorporated directly with the food. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may be between about 1 to about 75% of the weight of the unit. The amount of nucleic acids in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the nucleic acids in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. Dimethyl sulfoxide can be used as solvent for extremely rapid penetration, delivering high concentrations of the active agents to a small area.

The formulations of nucleic acids or lipid nanoparticles containing nucleic acids can be presented in unit-dose or multi-dose sealed containers, such as nebulizers, ventilators, ampules, and vials. Thus, the composition can be in unit dosage form. In such form the preparation is subdivided into unit doses containing appropriate quantities of nucleic acids or lipid nanoparticles containing nucleic acids. Thus, the compositions can be administered in a variety of unit dosage forms depending upon the method of administration. For example, unit dosage forms suitable for oral administration include, but are not limited to, powder, tablets, pills, capsules and lozenges.

The nucleic acids (e.g., RNA, sense RNA, sense RNA, antisense RNA, siRNA), lipid nanoparticles containing nucleic acids (e.g., RNA, sense RNA, sense RNA, antisense RNA, siRNA), and pharmaceutical compositions can be administered to the patient in any manner as described herein. In aspects, the disclosure provides a drug delivery device comprising the nucleic acids (e.g., RNA, sense RNA, sense RNA, antisense RNA, siRNA), lipid nanoparticles, or pharmaceutical compositions described herein. In aspects, the disclosure provides a nebulizer comprising the nucleic acids (e.g., RNA, sense RNA, sense RNA, antisense RNA, siRNA), lipid nanoparticles, or pharmaceutical compositions described herein. In aspects, the disclosure provides a syringe comprising the nucleic acids (e.g., RNA, sense RNA, sense RNA, antisense RNA, siRNA), lipid nanoparticles, or pharmaceutical compositions described herein. In aspects, the disclosure provides a ventilator comprising the nucleic acids (e.g., RNA, sense RNA, sense RNA, antisense RNA, siRNA), lipid nanoparticles, or pharmaceutical compositions described herein. In aspects, the disclosure provides a ventilator which comprises a nebulizer comprising the nucleic acids (e.g., RNA, sense RNA, sense RNA, antisense RNA, siRNA), lipid nanoparticles, or pharmaceutical compositions described herein. Drug delivery devices, such as nebulizers, ventilators, and syringes, are commercially available and well known in the art.

Methods of Treatment

In embodiments, the disclosure provides methods of treating COVID-19 in a subject in need thereof by administering to the subject an effective amount of a nucleic acid (e.g., RNA, sense RNA, sense RNA, antisense RNA, siRNA) that targets a nucleotide sequence of SARS-associated coronavirus 2. In embodiments, the disclosure provides methods of treating COVID-19 in a subject in need thereof by intranasally administering to the subject an effective amount of a nucleic acid (e.g., RNA, sense RNA, sense RNA, antisense RNA, siRNA) that targets a nucleotide sequence of SARS-associated coronavirus 2. In embodiments, the disclosure provides methods of treating COVID-19 in a subject in need thereof by intravenously administering to the subject an effective amount of a nucleic acid (e.g., RNA, sense RNA, sense RNA, antisense RNA, siRNA) that targets a nucleotide sequence of SARS-associated coronavirus 2.

In embodiments, the disclosure provides methods of treating severe acute respiratory syndrome (SARS) in a subject in need thereof by administering to the subject an effective amount of a nucleic acid (e.g., RNA, sense RNA, antisense RNA, siRNA) that targets a nucleotide sequence of SARS. In embodiments, the nucleic acid that targets a nucleotide sequence of SARS is any nucleic acid described herein. In embodiments, the nucleic acid that targets a nucleotide sequence of SARS is a sense strand described herein. In embodiments, the nucleic acid that targets a nucleotide sequence of SARS is any nucleic acid described herein. In embodiments, the nucleic acid that targets a nucleotide sequence of SARS is an antisense strand described herein. In embodiments, the nucleic acid that targets a nucleotide sequence of SARS is any nucleic acid described herein. In embodiments, the nucleic acid that targets a nucleotide sequence of SARS is a sense strand hybridized to an antisense strand described herein. In embodiments, the disclosure provides methods of treating severe acute respiratory syndrome (SARS) in a subject in need thereof by intranasally administering to the subject an effective amount of a nucleic acid that targets a nucleotide sequence of SARS. In embodiments, the disclosure provides methods of treating severe acute respiratory syndrome (SARS) in a subject in need thereof by intravenously administering to the subject an effective amount of a nucleic acid that targets a nucleotide sequence of SARS. In aspects, SARS is SARS-associated coronavirus. In aspects, SARS is SARS-associated coronavirus. In aspects, SARS is SARS-associated coronavirus 1 (SARS-CoV-1). In aspects, SARS is SARS-associated coronavirus 2 (SARS-CoV-2). In aspects, SARS is Middle Eastern respiratory syndrome coronavirus (MERS-CoV).

In embodiments, the disclosure provides methods of treating COVID-19 or SARS-associated coronavirus 2 (SARS-CoV-2) in a subject in need thereof, the method comprising administering to the subject an effective amount of the nucleic acids comprising at least 10 nucleotides and capable of hybridizing to any one of SEQ ID NOS:13-81; any one of SEQ ID NOS:100-124; any one of SEQ ID NOS:223-235, or any one of SEQ ID NOS:258-265. In aspects, the nucleic acid is capable of hybridizing to SEQ ID NO:224, SEQ ID NO:231, SEQ ID NO:124, SEQ ID NO:223, SEQ ID NO:225, SEQ ID NO:230, or SEQ ID NO:123. In aspects, the methods comprise nasally or intravenously administering an effective amount of the nucleic acids. In aspects, the methods comprise administering an effective amount of a lipid nanoparticle comprising a lipid and a nucleic acid. In aspects, the methods comprise intravenously or intranasally administering a lipid nanoparticle comprising a lipid and a nucleic acid. In aspects, the methods comprise administering a pharmaceutical composition comprising an pharmaceutically acceptable excipient and a lipid nanoparticle comprising a lipid and a nucleic acid. In aspects, the methods comprise intranasally or intravenously administering an effective amount of a pharmaceutical composition comprising an pharmaceutically acceptable excipient and a lipid nanoparticle comprising a lipid and a nucleic acid. In aspects, the methods comprise administering an effective amount of a pharmaceutical composition comprising an pharmaceutically acceptable excipient and a nucleic acid. In aspects, the methods comprise intranasally or intravenously administering an effective amount of a pharmaceutical composition comprising an pharmaceutically acceptable excipient and a nucleic acid. In aspects, the method is for treating COVID-19. In aspects, the method is for treating SARS-CoV-2.

In embodiments, the disclosure provides methods of treating COVID-19 or SARS-associated coronavirus 2 (SARS-CoV-2) in a subject in need thereof, the method comprising administering to the subject an effective amount of a nucleic acid described herein (e.g., a sense strand, an antisense strand, or a sense strand hybridized to an antisense strand). In embodiments, the nucleic acid is a sense strand as described herein. In embodiments, the nucleic acid is an antisense strand described herein. In embodiments, the nucleic acid is a sense strand hybridized to an antisense strand as described herein. In embodiments, the nucleic acid comprises SEQ ID NO:1, SEQ ID NO:3, SEQ ID NO:1 hybridized to SEQ ID NO:3, SEQ ID NO:2, SEQ ID NO:4, SEQ ID NO:2 hybridized to SEQ ID NO:4, SEQ ID NO:5, SEQ ID NO:7, SEQ ID NO:5 hybridized to SEQ ID NO:7, SEQ ID NO:6, SEQ ID NO:8, SEQ ID NO:6 hybridized to SEQ ID NO:8, SEQ ID NO:82, SEQ ID NO:83, SEQ ID NO:82 hybridized to SEQ ID NO:83, SEQ ID NO:84, SEQ ID NO:85, SEQ ID NO:84 hybridized to SEQ ID NO:85, SEQ ID NO:86, SEQ ID NO:87, SEQ ID NO:86 hybridized to SEQ ID NO:87 SEQ ID NO:88, SEQ ID NO:89, SEQ ID NO:88 hybridized to SEQ ID NO:89, SEQ ID NO:90, SEQ ID NO:91, SEQ ID NO:90 hybridized to SEQ ID NO:91, SEQ ID NO:92, SEQ ID NO:93, SEQ ID NO:92 hybridized to SEQ ID NO:93, SEQ ID NO:94, SEQ ID NO:95, SEQ ID NO:94 hybridized to SEQ ID NO:95, SEQ ID NO:96, SEQ ID NO:97, SEQ ID NO:96 hybridized to SEQ ID NO:97; SEQ ID NO:214 hybridized to SEQ ID NO:215; SEQ ID NO:214; SEQ ID NO:215; SEQ ID NO:216 hybridized to SEQ ID NO:217; SEQ ID NO:216; SEQ ID NO:217; SEQ ID NO:218 hybridized to SEQ ID NO:219; SEQ ID NO:218; SEQ ID NO:219; SEQ ID NO:220 hybridized to SEQ ID NO:221; SEQ ID NO:220; SEQ ID NO:221; SEQ ID NO:238 hybridized to SEQ ID NO:239; SEQ ID NO:238; SEQ ID NO:239; SEQ ID NO:240 hybridized to SEQ ID NO:241; SEQ ID NO:240; SEQ ID NO:241; SEQ ID NO:242 hybridized to SEQ ID NO:243; SEQ ID NO:242; SEQ ID NO:243; SEQ ID NO:244 hybridized to SEQ ID NO:245; SEQ ID NO:244; SEQ ID NO:245; SEQ ID NO:246 hybridized to SEQ ID NO:247; SEQ ID NO:246; SEQ ID NO:247; SEQ ID NO:248 hybridized to SEQ ID NO:249; SEQ ID NO:248; SEQ ID NO:249; SEQ ID NO:250 hybridized to SEQ ID NO:251; SEQ ID NO:250; SEQ ID NO:251; SEQ ID NO:252 hybridized to SEQ ID NO:253; SEQ ID NO:252; SEQ ID NO:253; SEQ ID NO:254 hybridized to SEQ ID NO:255; SEQ ID NO:254; SEQ ID NO:255; SEQ ID NO:256 hybridized to SEQ ID NO:257; SEQ ID NO:256; or SEQ ID NO:257. In aspects, the method comprises administering to the subject an effective amount of a sense strand in Table A, Table B, Table C, Table D, Table E, Table F, or Table G; an antisense strand in Table A, Table B, Table C, Table D, Table E, Table F, or Table G; or a sense strand in Table A, Table B, Table C, Table D, Table E, Table F, or Table G hybridized to the complementary antisense strand in Table A, Table B, Table C, Table D, Table E, Table F, or Table G, respectively. In aspects, the method comprises administering to the subject an effective amount of any one of SEQ ID NOS:266-273. In aspects, one or more nucleotides in the nucleic acids comprise a modified base, a modified sugar, a modified phosphate, or a combination of two or more thereof. In aspects, the method is for treating COVID-19. In aspects, the method is for treating SARS-CoV-2. In aspects, the methods comprise intranasal administration. In aspects, the methods comprise intranasal administration with a nebulizer. In aspects, the methods comprise intravenous administration.

In aspects, the methods comprise administering to the subject an effective amount of a lipid nanoparticle comprising a nucleic acid described herein (e.g., a sense strand, an antisense strand, or a sense strand hybridized to an antisense strand). In embodiments, the nucleic acid is a sense strand as described herein. In embodiments, the nucleic acid is an antisense strand described herein. In embodiments, the nucleic acid is a sense strand hybridized to an antisense strand as described herein. In embodiments, the lipid nanoparticle comprises a lipid and a nucleic acid comprising SEQ ID NO:1, SEQ ID NO:3, SEQ ID NO:1 hybridized to SEQ ID NO:3, SEQ ID NO:2, SEQ ID NO:4, SEQ ID NO:2 hybridized to SEQ ID NO:4, SEQ ID NO:5, SEQ ID NO:7, SEQ ID NO:5 hybridized to SEQ ID NO:7, SEQ ID NO:6, SEQ ID NO:8, SEQ ID NO:6 hybridized to SEQ ID NO:8, SEQ ID NO:82, SEQ ID NO:83, SEQ ID NO:82 hybridized to SEQ ID NO:83, SEQ ID NO:84, SEQ ID NO:85, SEQ ID NO:84 hybridized to SEQ ID NO:85, SEQ ID NO:86, SEQ ID NO:87, SEQ ID NO:86 hybridized to SEQ ID NO:87 SEQ ID NO:88, SEQ ID NO:89, SEQ ID NO:88 hybridized to SEQ ID NO:89, SEQ ID NO:90, SEQ ID NO:91, SEQ ID NO:90 hybridized to SEQ ID NO:91, SEQ ID NO:92, SEQ ID NO:93, SEQ ID NO:92 hybridized to SEQ ID NO:93, SEQ ID NO:94, SEQ ID NO:95, SEQ ID NO:94 hybridized to SEQ ID NO:95, SEQ ID NO:96, SEQ ID NO:97, SEQ ID NO:96 hybridized to SEQ ID NO:97; SEQ ID NO:214 hybridized to SEQ ID NO:215; SEQ ID NO:214; SEQ ID NO:215; SEQ ID NO:216 hybridized to SEQ ID NO:217; SEQ ID NO:216; SEQ ID NO:217; SEQ ID NO:218 hybridized to SEQ ID NO:219; SEQ ID NO:218; SEQ ID NO:219; SEQ ID NO:220 hybridized to SEQ ID NO:221; SEQ ID NO:220; SEQ ID NO:221; SEQ ID NO:238 hybridized to SEQ ID NO:239; SEQ ID NO:238; SEQ ID NO:239; SEQ ID NO:240 hybridized to SEQ ID NO:241; SEQ ID NO:240; SEQ ID NO:241; SEQ ID NO:242 hybridized to SEQ ID NO:243; SEQ ID NO:242; SEQ ID NO:243; SEQ ID NO:244 hybridized to SEQ ID NO:245; SEQ ID NO:244; SEQ ID NO:245; SEQ ID NO:246 hybridized to SEQ ID NO:247; SEQ ID NO:246; SEQ ID NO:247; SEQ ID NO:248 hybridized to SEQ ID NO:249; SEQ ID NO:248; SEQ ID NO:249; SEQ ID NO:250 hybridized to SEQ ID NO:251; SEQ ID NO:250; SEQ ID NO:251; SEQ ID NO:252 hybridized to SEQ ID NO:253; SEQ ID NO:252; SEQ ID NO:253; SEQ ID NO:254 hybridized to SEQ ID NO:255; SEQ ID NO:254; SEQ ID NO:255; SEQ ID NO:256 hybridized to SEQ ID NO:257; SEQ ID NO:256; or SEQ ID NO:257. In aspects, the method comprises administering to the subject an effective amount of a lipid nanoparticle comprising a lipid and a sense strand in Table A, Table B, Table C, Table D, Table E, Table F, or Table G; an antisense strand in Table A, Table B, Table C, Table D, Table E, Table F, or Table G; or a sense strand in Table A, Table B, Table C, Table D, Table E, Table F, or Table G hybridized to the complementary antisense strand in Table A, Table B, Table C, Table D, Table E, Table F, or Table G. In aspects, the method comprises administering to the subject an effective amount of a lipid nanoparticle comprising a lipid and any one of SEQ ID NOS:266-273. In aspects, one or more nucleotides in the nucleic acids comprise a modified base, a modified sugar, a modified phosphate, or a combination of two or more thereof. In aspects, the method is for treating COVID-19. In aspects, the method is for treating SARS-CoV-2. In aspects, the methods comprise intranasal administration. In aspects, the methods comprise intranasal administration with a nebulizer. In aspects, the methods comprise intravenous administration.

In aspects, the methods comprise administering an effective amount of a pharmaceutical composition comprising an pharmaceutically acceptable excipient and a lipid nanoparticle comprising a lipid and a nucleic acid described herein (e.g., a sense strand, an antisense strand, or a sense strand hybridized to an antisense strand). In embodiments, the nucleic acid is a sense strand as described herein. In embodiments, the nucleic acid is an antisense strand described herein. In embodiments, the nucleic acid is a sense strand hybridized to an antisense strand as described herein. In embodiments, the nucleic acid comprises SEQ ID NO:1, SEQ ID NO:3, SEQ ID NO:1 hybridized to SEQ ID NO:3, SEQ ID NO:2, SEQ ID NO:4, SEQ ID NO:2 hybridized to SEQ ID NO:4, SEQ ID NO:5, SEQ ID NO:7, SEQ ID NO:5 hybridized to SEQ ID NO:7, SEQ ID NO:6, SEQ ID NO:8, SEQ ID NO:6 hybridized to SEQ ID NO:8, SEQ ID NO:82, SEQ ID NO:83, SEQ ID NO:82 hybridized to SEQ ID NO:83, SEQ ID NO:84, SEQ ID NO:85, SEQ ID NO:84 hybridized to SEQ ID NO:85, SEQ ID NO:86, SEQ ID NO:87, SEQ ID NO:86 hybridized to SEQ ID NO:87 SEQ ID NO:88, SEQ ID NO:89, SEQ ID NO:88 hybridized to SEQ ID NO:89, SEQ ID NO:90, SEQ ID NO:91, SEQ ID NO:90 hybridized to SEQ ID NO:91, SEQ ID NO:92, SEQ ID NO:93, SEQ ID NO:92 hybridized to SEQ ID NO:93, SEQ ID NO:94, SEQ ID NO:95, SEQ ID NO:94 hybridized to SEQ ID NO:95, SEQ ID NO:96, SEQ ID NO:97, SEQ ID NO:96 hybridized to SEQ ID NO:97; SEQ ID NO:214 hybridized to SEQ ID NO:215; SEQ ID NO:214; SEQ ID NO:215; SEQ ID NO:216 hybridized to SEQ ID NO:217; SEQ ID NO:216; SEQ ID NO:217; SEQ ID NO:218 hybridized to SEQ ID NO:219; SEQ ID NO:218; SEQ ID NO:219; SEQ ID NO:220 hybridized to SEQ ID NO:221; SEQ ID NO:220; SEQ ID NO:221; SEQ ID NO:238 hybridized to SEQ ID NO:239; SEQ ID NO:238; SEQ ID NO:239; SEQ ID NO:240 hybridized to SEQ ID NO:241; SEQ ID NO:240; SEQ ID NO:241; SEQ ID NO:242 hybridized to SEQ ID NO:243; SEQ ID NO:242; SEQ ID NO:243; SEQ ID NO:244 hybridized to SEQ ID NO:245; SEQ ID NO:244; SEQ ID NO:245; SEQ ID NO:246 hybridized to SEQ ID NO:247; SEQ ID NO:246; SEQ ID NO:247; SEQ ID NO:248 hybridized to SEQ ID NO:249; SEQ ID NO:248; SEQ ID NO:249; SEQ ID NO:250 hybridized to SEQ ID NO:251; SEQ ID NO:250; SEQ ID NO:251; SEQ ID NO:252 hybridized to SEQ ID NO:253; SEQ ID NO:252; SEQ ID NO:253; SEQ ID NO:254 hybridized to SEQ ID NO:255; SEQ ID NO:254; SEQ ID NO:255; SEQ ID NO:256 hybridized to SEQ ID NO:257; SEQ ID NO:256; or SEQ ID NO:257. In aspects, the method comprises administering to the subject an effective amount of a pharmaceutical composition comprising an pharmaceutically acceptable excipient and a lipid nanoparticle comprising a lipid and a sense strand in Table A, Table B, Table C, Table D, Table E, Table F, or Table G; an antisense strand in Table A, Table B, Table C, Table D, Table E, Table F, or Table G; or a sense strand in Table A, Table B, Table C, Table D, Table E, Table F, or Table G hybridized to the complementary antisense strand in Table A, Table B, Table C, Table D, Table E, Table F, or Table G, respectively. In aspects, the method comprises administering to the subject an effective amount of a pharmaceutical composition comprising an pharmaceutically acceptable excipient and a lipid nanoparticle comprising a lipid and any one of SEQ ID NOS:266-273. In aspects, one or more nucleotides in the nucleic acids comprise a modified base, a modified sugar, a modified phosphate, or a combination of two or more thereof. In aspects, the method is for treating COVID-19. In aspects, the method is for treating SARS-CoV-2. In aspects, the methods comprise intranasal administration. In aspects, the methods comprise intranasal administration with a nebulizer. In aspects, the methods comprise intravenous administration.

In aspects, the methods comprise administering an effective amount of a pharmaceutical composition comprising an pharmaceutically acceptable excipient and a nucleic acid described herein (e.g., a sense strand, an antisense strand, or a sense strand hybridized to an antisense strand). In embodiments, the nucleic acid is a sense strand as described herein. In embodiments, the nucleic acid is an antisense strand described herein. In embodiments, the nucleic acid is a sense strand hybridized to an antisense strand as described herein. In embodiments, the nucleic acid comprises SEQ ID NO:1, SEQ ID NO:3, SEQ ID NO:1 hybridized to SEQ ID NO:3, SEQ ID NO:2, SEQ ID NO:4, SEQ ID NO:2 hybridized to SEQ ID NO:4, SEQ ID NO:5, SEQ ID NO:7, SEQ ID NO:5 hybridized to SEQ ID NO:7, SEQ ID NO:6, SEQ ID NO:8, SEQ ID NO:6 hybridized to SEQ ID NO:8, SEQ ID NO:82, SEQ ID NO:83, SEQ ID NO:82 hybridized to SEQ ID NO:83, SEQ ID NO:84, SEQ ID NO:85, SEQ ID NO:84 hybridized to SEQ ID NO:85, SEQ ID NO:86, SEQ ID NO:87, SEQ ID NO:86 hybridized to SEQ ID NO:87 SEQ ID NO:88, SEQ ID NO:89, SEQ ID NO:88 hybridized to SEQ ID NO:89, SEQ ID NO:90, SEQ ID NO:91, SEQ ID NO:90 hybridized to SEQ ID NO:91, SEQ ID NO:92, SEQ ID NO:93, SEQ ID NO:92 hybridized to SEQ ID NO:93, SEQ ID NO:94, SEQ ID NO:95, SEQ ID NO:94 hybridized to SEQ ID NO:95, SEQ ID NO:96, SEQ ID NO:97, SEQ ID NO:96 hybridized to SEQ ID NO:97; SEQ ID NO:214 hybridized to SEQ ID NO:215; SEQ ID NO:214; SEQ ID NO:215; SEQ ID NO:216 hybridized to SEQ ID NO:217; SEQ ID NO:216; SEQ ID NO:217; SEQ ID NO:218 hybridized to SEQ ID NO:219; SEQ ID NO:218; SEQ ID NO:219; SEQ ID NO:220 hybridized to SEQ ID NO:221; SEQ ID NO:220; SEQ ID NO:221; SEQ ID NO:238 hybridized to SEQ ID NO:239; SEQ ID NO:238; SEQ ID NO:239; SEQ ID NO:240 hybridized to SEQ ID NO:241; SEQ ID NO:240; SEQ ID NO:241; SEQ ID NO:242 hybridized to SEQ ID NO:243; SEQ ID NO:242; SEQ ID NO:243; SEQ ID NO:244 hybridized to SEQ ID NO:245; SEQ ID NO:244; SEQ ID NO:245; SEQ ID NO:246 hybridized to SEQ ID NO:247; SEQ ID NO:246; SEQ ID NO:247; SEQ ID NO:248 hybridized to SEQ ID NO:249; SEQ ID NO:248; SEQ ID NO:249; SEQ ID NO:250 hybridized to SEQ ID NO:251; SEQ ID NO:250; SEQ ID NO:251; SEQ ID NO:252 hybridized to SEQ ID NO:253; SEQ ID NO:252; SEQ ID NO:253; SEQ ID NO:254 hybridized to SEQ ID NO:255; SEQ ID NO:254; SEQ ID NO:255; SEQ ID NO:256 hybridized to SEQ ID NO:257; SEQ ID NO:256; or SEQ ID NO:257. In aspects, the method comprises administering to the subject an effective amount of a pharmaceutical composition comprising an pharmaceutically acceptable excipient and a sense strand in Table A, Table B, Table C, Table D, or Table E; an antisense strand in Table A, Table B, Table C, Table D, or Table E; or a sense strand in Table A, Table B, Table C, Table D, or Table E hybridized to the complementary antisense strand in Table A, Table B, Table C, Table D, or Table E, respectively. In aspects, the method comprises administering to the subject an effective amount of a pharmaceutical composition comprising a pharmaceutically acceptable excipient and any one of SEQ ID NOS:266-273. In aspects, one or more nucleotides in the nucleic acids comprise a modified base, a modified sugar, a modified phosphate, or a combination of two or more thereof. In aspects, the method is for treating COVID-19. In aspects, the method is for treating SARS-CoV-2. In aspects, the methods comprise intranasal administration. In aspects, the methods comprise intranasal administration with a nebulizer. In aspects, the methods comprise intravenous administration.

In embodiments, the disclosure provides methods of treating SARS-associated coronavirus 1 (SARS-CoV-1) in a subject in need thereof, the method comprising administering to the subject an effective amount of the nucleic acids comprising at least 10 nucleotides and capable of hybridizing to any one of SEQ ID NOS:50-53 and 78-81. In aspects, the methods comprise administering an effective amount of a lipid nanoparticle comprising a lipid and a nucleic acid. In aspects, the methods comprise intravenously or intranasally administering a lipid nanoparticle comprising a lipid and a nucleic acid. In aspects, the methods comprise administering a pharmaceutical composition comprising an pharmaceutically acceptable excipient and a lipid nanoparticle comprising a lipid and a nucleic acid. In aspects, the methods comprise intranasally or intravenously administering an effective amount of a pharmaceutical composition comprising an pharmaceutically acceptable excipient and a lipid nanoparticle comprising a lipid and a nucleic acid. In aspects, the methods comprise administering an effective amount of a pharmaceutical composition comprising an pharmaceutically acceptable excipient and a nucleic acid. In aspects, the methods comprise intranasally or intravenously administering an effective amount of a pharmaceutical composition comprising an pharmaceutically acceptable excipient and a nucleic acid. In aspects, the nucleic acid is capable of hybridizing to SEQ ID NO:50. In aspects, the nucleic acid is capable of hybridizing to SEQ ID NO:51. In aspects, the nucleic acid is capable of hybridizing to SEQ ID NO:52. In aspects, the nucleic acid is capable of hybridizing to SEQ ID NO:53. In aspects, the nucleic acid is capable of hybridizing to SEQ ID NO:78. In aspects, the nucleic acid is capable of hybridizing to SEQ ID NO:79. In aspects, the nucleic acid is capable of hybridizing to SEQ ID NO:80. In aspects, the nucleic acid is capable of hybridizing to SEQ ID NO:81. In aspects, one or more nucleotides in the nucleic acids comprise a modified base, a modified sugar, a modified phosphate, or a combination of two or more thereof. In aspects, the methods comprise intranasal administration. In aspects, the methods comprise intranasal administration with a nebulizer. In aspects, the methods comprise intravenous administration.

In embodiments, the disclosure provides methods of treating SARS by administering an effective amount of a nucleic acid comprising a sense strand in Table A, Table B, Table C, Table D, Table E, Table F, or Table G, an antisense strand in Table A, Table B, Table C, Table D, Table E, Table F, or Table G, or a nucleic acid comprising a sense strand in Table A, Table B, Table C, Table D, Table E, Table F, or Table G hybridized to the complementary antisense strand in Table A, Table B, Table C, Table D, Table E, Table F, or Table G, respectively. In aspects, SARS is SARS-associated coronavirus. In aspects, SARS is SARS-associated coronavirus 1. In aspects, SARS is SARS-associated coronavirus 2. In aspects, SARS is MERS-associated coronavirus. The nucleic acids can be modified as described herein. The nucleic acids can be in the form of nanoparticles and pharmaceutical compositions as described herein.

In embodiments, the disclosure provides methods of treating SARS by administering an effective amount of a nucleic acid comprising any one of SEQ ID NOS:266-273. The nucleic acids can be modified as described herein. The nucleic acids can be in the form of nanoparticles and pharmaceutical compositions as described herein. In embodiments, SARS is COVID-19. In embodiments, SARS is SARS-CoV. In embodiments, SARS is SARS-CoV-1. In embodiments, SARS is SARS-CoV-2. In embodiments, SARS is MERS. In embodiments, SARS is Middle Eastern respiratory syndrome (MERS).

Dose and Dosing Regimens

The dosage and frequency (single or multiple doses) of the nucleic acids (e.g., RNA, sense RNA, antisense RNA, siRNA) administered to a subject can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and nucleic acids (e.g., RNA, sense RNA, antisense RNA, siRNA) described herein. Adjustment and manipulation of established dosages (e.g., frequency and duration) are within the ability of the skilled artisan.

For any composition and nucleic acids (e.g., RNA, sense RNA, antisense RNA, siRNA) described herein, the effective amount can be initially determined from cell culture assays. Target concentrations will be those concentrations of nucleic acids (e.g., RNA, sense RNA, antisense RNA, siRNA) that are capable of achieving the methods described herein, as measured using the methods described herein or known in the art. As is known in the art, effective amounts of nucleic acids (e.g., RNA, sense RNA, antisense RNA, siRNA) for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages of the nucleic acids (e.g., RNA, sense RNA, antisense RNA, siRNA) may be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Dosage amounts and intervals can be adjusted individually to provide levels of the nucleic acids (e.g., RNA, sense RNA, antisense RNA, siRNA) effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical disease or symptoms demonstrated by the particular patient. This planning should involve the careful choice of nucleic acids (e.g., RNA, sense RNA, antisense RNA, siRNA) by considering factors such as compound potency, relative bioavailability, patient body weight, presence and severity of adverse side effects.

In embodiments, the nucleic acid (e.g., RNA, sense RNA, antisense RNA, siRNA) is administered to a patient at an amount of about 0.001 mg/kg to about 500 mg/kg. In aspects, the nucleic acids is administered to a patient in an amount of about 0.01 mg/kg, 0.1 mg/kg, 0.5 mg/kg, 1 mg/kg, 2 mg/kg, 3 mg/kg, 4 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, or 300 mg/kg. It is understood that where the amount is referred to as “mg/kg,” the amount is milligram per kilogram body weight of the subject being administered with the nucleic acid (e.g., RNA, sense RNA, antisense RNA, siRNA). In aspects, the nucleic acid (e.g., RNA, sense RNA, antisense RNA, siRNA) is administered to a patient in an amount from about 0.01 mg to about 500 mg per day, as a single dose, or in a dose administered two or three times per day.

### Embodiments N1 to N88

Embodiment N1. A nucleic acid comprising 15 nucleotides to 30 nucleotides and capable of hybridizing to SEQ ID NO:224, SEQ ID NO:231, SEQ ID NO:124, SEQ ID NO:223, SEQ ID NO:225, SEQ ID NO:230, or SEQ ID NO:123.

Embodiment N2. The nucleic acid of Embodiment N1, comprising 20 nucleotides to 25 nucleotides.

Embodiment N3. A nucleic acid comprising SEQ ID NO:1 hybridized to SEQ ID NO:3.

Embodiment N4. A nucleic acid comprising SEQ ID NO:2 hybridized to SEQ ID NO:4.

Embodiment N5. A nucleic acid comprising SEQ ID NO:250 hybridized to SEQ ID NO:251.

Embodiment N6. A nucleic acid comprising SEQ ID NO:220 hybridized to SEQ ID NO:221.

Embodiment N7. A nucleic acid comprising SEQ ID NO:5 hybridized to SEQ ID NO:7.

Embodiment N8. A nucleic acid comprising SEQ ID NO:6 hybridized to SEQ ID NO:8.

Embodiment N9. A nucleic acid comprising SEQ ID NO:238 hybridized to SEQ ID NO:239.

Embodiment N10. A nucleic acid comprising SEQ ID NO:248 hybridized to SEQ ID NO:249.

Embodiment N11. A nucleic acid comprising SEQ ID NO:218 hybridized to SEQ ID NO:219.

Embodiment N12. A nucleic acid comprising SEQ ID NO:1 or SEQ ID NO:3.

Embodiment N13. A nucleic acid comprising SEQ ID NO:2 or SEQ ID NO:4.

Embodiment N14. A nucleic acid comprising SEQ ID NO:5 or SEQ ID NO:7.

Embodiment N15. A nucleic acid comprising SEQ ID NO:6 or SEQ ID NO:8.

Embodiment N16. A nucleic acid comprising SEQ ID NO:250 or SEQ ID NO:251.

Embodiment N17. A nucleic acid comprising SEQ ID NO:220 or SEQ ID NO:221.

Embodiment N18. A nucleic acid comprising SEQ ID NO:238 or SEQ ID NO:239.

Embodiment N19. A nucleic acid comprising SEQ ID NO:248 or SEQ ID NO:249.

Embodiment N20. A nucleic acid comprising SEQ ID NO:218 or SEQ ID NO:219.

Embodiment N21. A nucleic acid comprising SEQ ID NO:82 hybridized to SEQ ID NO:83; SEQ ID NO:82; or SEQ ID NO:83.

Embodiment N22. A nucleic acid comprising SEQ ID NO:84 hybridized to SEQ ID NO:85; SEQ ID NO:84; or SEQ ID NO:85.

Embodiment N23. A nucleic acid comprising SEQ ID NO:86 hybridized to SEQ ID NO:87; SEQ ID NO:86; or SEQ ID NO:87.

Embodiment N24. A nucleic acid comprising SEQ ID NO:88 hybridized to SEQ ID NO:89; SEQ ID NO:88; or SEQ ID NO:89.

Embodiment N25. A nucleic acid comprising SEQ ID NO:90 hybridized to SEQ ID NO:91; SEQ ID NO:90; or SEQ ID NO:91.

Embodiment N26. A nucleic acid comprising SEQ ID NO:92 hybridized to SEQ ID NO:93; SEQ ID NO:92; or SEQ ID NO:93.

Embodiment N27. A nucleic acid comprising SEQ ID NO:94 hybridized to SEQ ID NO:95; SEQ ID NO:94; or SEQ ID NO:95.

Embodiment N28. A nucleic acid comprising SEQ ID NO:96 hybridized to SEQ ID NO:97; SEQ ID NO:96; or SEQ ID NO:97.

Embodiment N29. A nucleic acid comprising SEQ ID NO:214 hybridized to SEQ ID NO:215; SEQ ID NO:214; or SEQ ID NO:215.

Embodiment N30. A nucleic acid comprising SEQ ID NO:216 hybridized to SEQ ID NO:217; SEQ ID NO:216; or SEQ ID NO:217.

Embodiment N31. A nucleic acid comprising SEQ ID NO:236 hybridized to SEQ ID NO:237; SEQ ID NO:236; or SEQ ID NO:237.

Embodiment N32. A nucleic acid comprising SEQ ID NO:240 hybridized to SEQ ID NO:241; SEQ ID NO:240; or SEQ ID NO:241.

Embodiment N33. A nucleic acid comprising SEQ ID NO:242 hybridized to SEQ ID NO:243; SEQ ID NO:242; or SEQ ID NO:243.

Embodiment N34. A nucleic acid comprising SEQ ID NO:244 hybridized to SEQ ID NO:245; SEQ ID NO:244; or SEQ ID NO:245.

Embodiment N35. A nucleic acid comprising SEQ ID NO:246 hybridized to SEQ ID NO:247; SEQ ID NO:246; or SEQ ID NO:247.

Embodiment N36. A nucleic acid comprising SEQ ID NO:252 hybridized to SEQ ID NO:253; SEQ ID NO:252; or SEQ ID NO:253.

Embodiment N37. A nucleic acid comprising SEQ ID NO:254 hybridized to SEQ ID NO:255; SEQ ID NO:254; or SEQ ID NO:255.

Embodiment N38. A nucleic acid comprising SEQ ID NO:256 hybridized to SEQ ID NO:257; SEQ ID NO:256; or SEQ ID NO:257.

Embodiment N39. A nucleic acid comprising SEQ ID NO:125 hybridized to SEQ ID NO:126; SEQ ID NO:126 hybridized to SEQ ID NO:127; SEQ ID NO:104 hybridized to SEQ ID NO:128; SEQ ID NO:129 hybridized to SEQ ID NO:130; SEQ ID NO:131 hybridized to SEQ ID NO:132; SEQ ID NO:133 hybridized to SEQ ID NO:134; SEQ ID NO:135 hybridized to SEQ ID NO:136; SEQ ID NO:137 hybridized to SEQ ID NO:138; SEQ ID NO:139 hybridized to SEQ ID NO:140; SEQ ID NO:141 hybridized to SEQ ID NO:142; SEQ ID NO:143 hybridized to SEQ ID NO:144; SEQ ID NO:145 hybridized to SEQ ID NO:146; SEQ ID NO:147 hybridized to SEQ ID NO:148; SEQ ID NO:149 hybridized to SEQ ID NO:150; SEQ ID NO:115 hybridized to SEQ ID NO:151; SEQ ID NO:152 hybridized to SEQ ID NO:153; SEQ ID NO:154 hybridized to SEQ ID NO:155; SEQ ID NO:156 hybridized to SEQ ID NO:157; SEQ ID NO:158 hybridized to SEQ ID NO:159; SEQ ID NO:160 hybridized to SEQ ID NO:161; or SEQ ID NO:162 hybridized to SEQ ID NO:163.

Embodiment N40. A nucleic acid comprising SEQ ID NO:125; SEQ ID NO:126; SEQ ID NO:126; SEQ ID NO:127; SEQ ID NO:104; SEQ ID NO:128; SEQ ID NO:129; SEQ ID NO:130; SEQ ID NO:131; SEQ ID NO:132; SEQ ID NO:133; SEQ ID NO:134; SEQ ID NO:135; SEQ ID NO:136; SEQ ID NO:137; SEQ ID NO:138; SEQ ID NO:139; SEQ ID NO:140; SEQ ID NO:141; SEQ ID NO:142; SEQ ID NO:143; SEQ ID NO:144; SEQ ID NO:145; SEQ ID NO:146; SEQ ID NO:147; SEQ ID NO:148; SEQ ID NO:149; SEQ ID NO:150; SEQ ID NO:115; SEQ ID NO:151; SEQ ID NO:152; SEQ ID NO:153; SEQ ID NO:154; SEQ ID NO:155; SEQ ID NO:156; SEQ ID NO:157; SEQ ID NO:158; SEQ ID NO:159; SEQ ID NO:160; SEQ ID NO:161; SEQ ID NO:162; or SEQ ID NO:163.

Embodiment N41. A nucleic acid comprising SEQ ID NO:164 hybridized to SEQ ID NO:165; SEQ ID NO:166 hybridized to SEQ ID NO:167; SEQ ID NO:168 hybridized to SEQ ID NO:169; SEQ ID NO:170 hybridized to SEQ ID NO:171; SEQ ID NO:172 hybridized to SEQ ID NO:173; SEQ ID NO:174 hybridized to SEQ ID NO:175; SEQ ID NO:176 hybridized to SEQ ID NO:177; SEQ ID NO:178 hybridized to SEQ ID NO:179; SEQ ID NO:180 hybridized to SEQ ID NO:181; SEQ ID NO:182 hybridized to SEQ ID NO:183; SEQ ID NO:184 hybridized to SEQ ID NO:185; SEQ ID NO:186 hybridized to SEQ ID NO:187; SEQ ID NO:188 hybridized to SEQ ID NO:189; SEQ ID NO:190 hybridized to SEQ ID NO:191; SEQ ID NO:192 hybridized to SEQ ID NO:193; SEQ ID NO:194 hybridized to SEQ ID NO:195; SEQ ID NO:196 hybridized to SEQ ID NO:197; SEQ ID NO:198 hybridized to SEQ ID NO:199; SEQ ID NO:200 hybridized to SEQ ID NO:201; SEQ ID NO:202 hybridized to SEQ ID NO:203; or SEQ ID NO:204 hybridized to SEQ ID NO:205.

Embodiment N42. A nucleic acid comprising SEQ ID NO:164; SEQ ID NO:165; SEQ ID NO:166; SEQ ID NO:167; SEQ ID NO:168; SEQ ID NO:169; SEQ ID NO:170; SEQ ID NO:171; SEQ ID NO:172; SEQ ID NO:173; SEQ ID NO:174; SEQ ID NO:175; SEQ ID NO:176; SEQ ID NO:177; SEQ ID NO:178; SEQ ID NO:179; SEQ ID NO:180; SEQ ID NO:181; SEQ ID NO:182; SEQ ID NO:183; SEQ ID NO:184; SEQ ID NO:185; SEQ ID NO:186; SEQ ID NO:187; SEQ ID NO:188; SEQ ID NO:189; SEQ ID NO:190; SEQ ID NO:191; SEQ ID NO:192; SEQ ID NO:193; SEQ ID NO:194; SEQ ID NO:195; SEQ ID NO:196; SEQ ID NO:197; SEQ ID NO:198; SEQ ID NO:199; SEQ ID NO:200; SEQ ID NO:201; SEQ ID NO:202; SEQ ID NO:203; SEQ ID NO:204; or SEQ ID NO:205.

Embodiment N43. A nucleic acid comprising SEQ ID NO:206 hybridized to SEQ ID NO:207; SEQ ID NO:208 hybridized to SEQ ID NO:209; SEQ ID NO:210 hybridized to SEQ ID NO:211; or SEQ ID NO:212 hybridized to SEQ ID NO:213.

Embodiment N44. A nucleic acid comprising SEQ ID NO:206; SEQ ID NO:207; SEQ ID NO:208; SEQ ID NO:209; SEQ ID NO:210; SEQ ID NO:211; SEQ ID NO:212; or SEQ ID NO:213.

Embodiment N45. A nucleic acid comprising at least 10 nucleotides and capable of hybridizing to any one of SEQ ID NOS:13-81; any one of SEQ ID NOS:100-124; any one of SEQ ID NOS:223-235, or any one of SEQ ID NOS:258-265.

Embodiment N46. The nucleic acid of Embodiment N45, comprising 15 nucleotides to 30 nucleotides.

Embodiment N47. The nucleic acid of Embodiment N46, comprising 20 nucleotides to 25 nucleotides.

Embodiment N48. The nucleic acid of Embodiment N47, comprising 21 nucleotides to 23 nucleotides.

Embodiment N49. A nucleic acid having at least 80% sequence identify to SEQ ID NO:266, SEQ ID NO:267, SEQ ID NO:268, SEQ ID NO:269, SEQ ID NO:270, SEQ ID NO:271, SEQ ID NO:272, or SEQ ID NO:273.

Embodiment N50. The nucleic acid of Embodiment N49 having at least 90% sequence identify to SEQ ID NO:266, SEQ ID NO:267, SEQ ID NO:268, SEQ ID NO:269, SEQ ID NO:270, SEQ ID NO:271, SEQ ID NO:272, or SEQ ID NO:273.

Embodiment NM. The nucleic acid of Embodiment N50 comprising SEQ ID NO:266, SEQ ID NO:267, SEQ ID NO:268, SEQ ID NO:269, SEQ ID NO:270, SEQ ID NO:271, SEQ ID NO:272, or SEQ ID NO:273.

Embodiment N52. The nucleic acid of any one of Embodiments N1 to NM, wherein the nucleic acid sequence comprises a modified base, a modified sugar, a modified phosphate, or a combination of two or more thereof.

Embodiment N53. The nucleic acid of Embodiment N52, wherein the modified base is 2′O-Methyl modified base, a 2′O-methoxyethoxy modified base, a 2′fluoro modified base, a 5-methyl-modified cytidine, or pseudouridine.

Embodiment N54. The nucleic acid of Embodiment N53, wherein the modified base is a 2′O-Methyl modified base.

Embodiment N55. The nucleic acid of any one of Embodiments N52 to N54, wherein the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite.

Embodiment N56. The nucleic acid of Embodiment N55, wherein the modified phosphate is phosphorothioate.

Embodiment N57. The nucleic acid of any one of Embodiments N52 to N56, wherein the modified sugar is deoxyribose.

Embodiment N58. The nucleic acid of any one of Embodiments N1 to N57, wherein the nucleic acid is RNA.

Embodiment N59. The nucleic acid of any one of Embodiments N1 to N57, wherein the nucleic acid is antisense RNA.

Embodiment N60. The nucleic acid of any one of Embodiments N1 to N57, wherein the nucleic acid is siRNA.

Embodiment N61. A lipid nanoparticle comprising a lipid and the nucleic acid of any one of Embodiments N1 to N60.

Embodiment N62. The lipid nanoparticle of Embodiment N61, wherein the lipid comprises a cationic lipid, a non-cationic lipid, a conjugated lipid that prevents aggregation of the nanoparticle, or a combination of two or more thereof.

Embodiment N63. A pharmaceutical composition comprising the lipid nanoparticle of Embodiment N61 or N62 and a pharmaceutically acceptable excipient.

Embodiment N64. A pharmaceutical composition comprising the nucleic acid of any one of Embodiments N1 to N60 and a pharmaceutically acceptable excipient.

Embodiment N65. The pharmaceutical composition of Embodiment N63 or N64, wherein the composition is an aerosol.

Embodiment N66. The pharmaceutical composition of Embodiment N63 or N64, wherein the composition is a liquid.

Embodiment N67. A method of treating COVID-19 in a subject in need thereof, the method comprising administering to the subject an effective amount of the nucleic acid of any one of Embodiments N1 to N60.

Embodiment N68. A method of treating COVID-19 in a subject in need thereof, the method comprising administering to the subject an effective amount of the lipid nanoparticle of Embodiment N61 or N62.

Embodiment N69. A method of treating COVID-19 in a subject in need thereof, the method comprising administering to the subject an effective amount of the pharmaceutical composition of any one of Embodiments N63 to N66.

Embodiment N70. A method of treating SARS-associated coronavirus 2 (SARS-CoV-2) in a subject in need thereof, the method comprising administering to the subject an effective amount of the nucleic acid of any one of Embodiments N1 to N60.

Embodiment N71. A method of treating SARS-associated coronavirus 2 (SARS-CoV-2) in a subject in need thereof, the method comprising administering to the subject an effective amount of the lipid nanoparticle of Embodiment N61 or N62.

Embodiment N72. A method of treating SARS-associated coronavirus 2 (SARS-CoV-2) in a subject in need thereof, the method comprising administering to the subject an effective amount of the pharmaceutical composition of any one of Embodiments N63 to N66.

Embodiment N73. A method of treating severe acute respiratory syndrome in a subject in need thereof, the method comprising administering to the subject an effective amount of the nucleic acid of any one of Embodiments N1 to N60.

Embodiment N74. A method of treating severe acute respiratory syndrome in a subject in need thereof, the method comprising administering to the subject an effective amount of the lipid nanoparticle of Embodiment N61 or N62.

Embodiment N75. A method of treating severe acute respiratory syndrome in a subject in need thereof, the method comprising administering to the subject an effective amount of the pharmaceutical composition of any one of Embodiments N63 to N66.

Embodiment N76. The method of any one of Embodiments N73 to N75, wherein the severe acute respiratory syndrome is SARS-associated coronavirus (SARS-CoV).

Embodiment N77. The method of any one of Embodiments 73 to 75, wherein the severe acute respiratory syndrome is SARS-associated coronavirus 1 (SARS-CoV-1).

Embodiment N78. The method of any one of Embodiments N73 to N75, wherein the severe acute respiratory syndrome is MERS-associated coronavirus (MERS-CoV).

Embodiment N79. A method of treating Middle Eastern respiratory syndrome in a subject in need thereof, the method comprising administering to the subject an effective amount of the nucleic acid of any one of Embodiments N1 to N60.

Embodiment N80. A method of treating Middle Eastern respiratory syndrome in a subject in need thereof, the method comprising administering to the subject an effective amount of the lipid nanoparticle of Embodiment N61 or N62.

Embodiment N81. A method of treating Middle Eastern respiratory syndrome in a subject in need thereof, the method comprising administering to the subject an effective amount of the pharmaceutical composition of any one of Embodiments N63 to N66.

Embodiment N82. The method of any one of Embodiments N67 to N81, comprising nasally administering.

Embodiment N83. The method of any one of Embodiments N67 to N81, comprising intravenously administering.

Embodiment N84. A drug delivery device comprising an effective amount of the nucleic acid of any one of Embodiments N1 to N60.

Embodiment N85. A drug delivery device comprising an effective amount of the lipid nanoparticle of Embodiment N61 or N62.

Embodiment N86. A drug delivery device comprising an effective amount of the pharmaceutical composition of any one of Embodiments N63 to N66.

Embodiment N87. The method of any one of Embodiments N84 to N86, wherein the drug delivery device is a nebulizer.

Embodiment N88. The method of any one of Embodiments N84 to N86, wherein the drug delivery device is a syringe.

### Embodiments 1 to 83

Embodiment 1. A nucleic acid comprising at least 10 nucleotides and capable of hybridizing to any one of SEQ ID NOS:29-49.

Embodiment 2. A nucleic acid comprising at least 10 nucleotides and capable of hybridizing to any one of SEQ ID NOS:13-20 and 26-28.

Embodiment 3. A nucleic acid comprising at least 10 nucleotides and capable of hybridizing to any one of SEQ ID NOS:57-77.

Embodiment 4. A nucleic acid comprising at least 10 nucleotides and capable of hybridizing to any one of SEQ ID NOS:21-25 and 54-56.

Embodiment 5. A nucleic acid comprising at least 10 nucleotides and capable of hybridizing to any one of SEQ ID NOS:50-53.

Embodiment 6. A nucleic acid comprising at least 10 nucleotides and capable of hybridizing to any one of SEQ ID NOS:78-81.

Embodiment 7. The nucleic acid of any one of Embodiments 1 to 6, comprising 15 nucleotides or more.

Embodiment 8. The nucleic acid of Embodiment 7, comprising from about 20 nucleotides to about 25 nucleotides.

Embodiment 9. The nucleic acid of Embodiment 8, comprising from about 21 nucleotides to about 23 nucleotides.

Embodiment 10. The nucleic acid of Embodiment 9, comprising 21 nucleotides.

Embodiment 11. The nucleic acid of Embodiment 9, comprising 22 nucleotides.

Embodiment 12. The nucleic acid of Embodiment 9, comprising 23 nucleotides.

Embodiment 13. A nucleic acid comprising SEQ ID NO:1 hybridized to SEQ ID NO:3.

Embodiment 14. A nucleic acid comprising SEQ ID NO:1.

Embodiment 15. A nucleic acid comprising SEQ ID NO:3.

Embodiment 16. A nucleic acid comprising SEQ ID NO:5 hybridized to SEQ ID NO:7.

Embodiment 17. A nucleic acid comprising SEQ ID NO:5.

Embodiment 18. A nucleic acid comprising SEQ ID NO:7.

Embodiment 19. A nucleic acid comprising SEQ ID NO:82 hybridized to SEQ ID NO:83.

Embodiment 20. A nucleic acid comprising SEQ ID NO:82.

Embodiment 21. A nucleic acid comprising SEQ ID NO:83.

Embodiment 22. A nucleic acid comprising SEQ ID NO:84 hybridized to SEQ ID NO:85.

Embodiment 23. A nucleic acid comprising SEQ ID NO:84.

Embodiment 24. A nucleic acid comprising SEQ ID NO:85.

Embodiment 25. A nucleic acid comprising SEQ ID NO:86 hybridized to SEQ ID NO:87.

Embodiment 26. A nucleic acid comprising SEQ ID NO:86.

Embodiment 27. A nucleic acid comprising SEQ ID NO:87.

Embodiment 28. A nucleic acid comprising SEQ ID NO:88 hybridized to SEQ ID NO:89.

Embodiment 29. A nucleic acid comprising SEQ ID NO:88.

Embodiment 30. A nucleic acid comprising SEQ ID NO:89.

Embodiment 31. A nucleic acid comprising SEQ ID NO:90 hybridized to SEQ ID NO:91.

Embodiment 32. A nucleic acid comprising SEQ ID NO:90.

Embodiment 33. A nucleic acid comprising SEQ ID NO:91.

Embodiment 34. A nucleic acid comprising SEQ ID NO:92 hybridized to SEQ ID NO:93.

Embodiment 35. A nucleic acid comprising SEQ ID NO:92.

Embodiment 36. A nucleic acid comprising SEQ ID NO:93.

Embodiment 37. A nucleic acid comprising SEQ ID NO:94 hybridized to SEQ ID NO:95.

Embodiment 38. A nucleic acid comprising SEQ ID NO:94.

Embodiment 39. A nucleic acid comprising SEQ ID NO:95.

Embodiment 40. A nucleic acid comprising SEQ ID NO:96 hybridized to SEQ ID NO:97.

Embodiment 41. A nucleic acid comprising SEQ ID NO:96.

Embodiment 42. A nucleic acid comprising SEQ ID NO:97.

Embodiment 43. A nucleic acid comprising a sense strand from Table A; an antisense strand from Table A; or a sense strand from Table A hybridized to a complementary antisense strand from Table A.

Embodiment 44. A nucleic acid comprising a sense strand from Table B; an antisense strand from Table B; or a sense strand from Table B hybridized to a complementary antisense strand from Table B.

Embodiment 45. A nucleic acid comprising a sense strand from Table C; an antisense strand from Table C; or a sense strand from Table C hybridized to a complementary antisense strand from Table C.

Embodiment 46. A nucleic acid comprising a sense strand from Table D; an antisense strand from Table D; or a sense strand from Table D hybridized to a complementary antisense strand from Table D.

Embodiment 47. The nucleic acid of any one of Embodiments 1 to 46, wherein the nucleic acid sequence comprises a modified base, a modified sugar, a modified phosphate, or a combination of two or more thereof.

Embodiment 48. The nucleic acid of Embodiment 47, wherein the modified base is 2′O-Methyl modified base, a 2′O-methoxyethoxy modified base, a 2′fluoro modified base, a 5-methyl-modified cytidine, or pseudouridine.

Embodiment 49. The nucleic acid of Embodiment 48, wherein the modified base is a 2′O-Methyl modified base.

Embodiment 50. The nucleic acid of any one of Embodiments 47 to 49, wherein the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite.

Embodiment 51. The nucleic acid of Embodiment 50, wherein the modified phosphate is phosphorothioate.

Embodiment 52. The nucleic acid of any one of Embodiments 47 to 51, wherein the modified sugar is deoxyribose.

Embodiment 53. A nucleic acid comprising SEQ ID NO:2 hybridized to SEQ ID NO:4.

Embodiment 54. A nucleic acid comprising SEQ ID NO:2.

Embodiment 55. A nucleic acid comprising SEQ ID NO:4.

Embodiment 56. A nucleic acid comprising SEQ ID NO:6 hybridized to SEQ ID NO:8.

Embodiment 57. A nucleic acid comprising SEQ ID NO:6.

Embodiment 58. A nucleic acid comprising SEQ ID NO:8.

Embodiment 59. The nucleic acid of any one of Embodiments 1 to 58, wherein the nucleic acid is RNA.

Embodiment 60. The nucleic acid of any one of Embodiments 1 to 58, wherein the nucleic acid is antisense RNA.

Embodiment 61. The nucleic acid of any one of Embodiments 1 to 58, wherein the nucleic acid is siRNA.

Embodiment 62. A lipid nanoparticle comprising a lipid and the nucleic acid of any one of Embodiments 1 to 61.

Embodiment 63. The lipid nanoparticle of Embodiment 62, wherein the lipid comprises a cationic lipid, a non-cationic lipid, a conjugated lipid that prevents aggregation of the nanoparticle, or a combination of two or more thereof.

Embodiment 64. The lipid nanoparticle of Embodiment 62, wherein the lipid comprises N-(1-(2,3-dioleoyloxy)propyl)-N,N,N-trimethylammonium chloride, cholesterol, dioleoylphosphatidyl-ethanolamine, polyethylene glycol conjugated to ceramide, or a combination of two or more thereof.

Embodiment 65. A pharmaceutical composition comprising the lipid nanoparticle of any one of Embodiments 62 to 64 and a pharmaceutically acceptable excipient.

Embodiment 66. A pharmaceutical composition comprising the nucleic acid of any one of Embodiments 1 to 61 and a pharmaceutically acceptable excipient.

Embodiment 67. The pharmaceutical composition of Embodiment 65 or 66, wherein the composition is an aerosol.

Embodiment 68. The pharmaceutical composition of Embodiment 65 or 66, wherein the composition is a liquid.

Embodiment 69. A method of treating COVID-19 in a subject in need thereof, the method comprising administering to the subject an effective amount of the nucleic acid of any one of Embodiments 1-61; the lipid nanoparticle of any one of Embodiments 62 to 64; or the pharmaceutical composition of any one of Embodiments 65 to 68.

Embodiment 70. A method of treating SARS-associated coronavirus 2 in a subject in need thereof, the method comprising administering to the subject an effective amount of the nucleic acid of any one of Embodiments 1-61; the lipid nanoparticle of any one of Embodiments 62 to 64; or the pharmaceutical composition of any one of Embodiments 65 to 68.

Embodiment 71. A method of treating severe acute respiratory syndrome in a subject in need thereof, the method comprising administering to the subject an effective amount of the nucleic acid of any one of Embodiments 1-61; the lipid nanoparticle of any one of Embodiments 62 to 64; or the pharmaceutical composition of any one of Embodiments 65 to 68.

Embodiment 72. The method of Embodiment 71, wherein the severe acute respiratory syndrome is SARS-associated coronavirus.

Embodiment 73. The method of Embodiment 71, wherein the severe acute respiratory syndrome is SARS-associated coronavirus 1.

Embodiment 74. The method of Embodiment 71, wherein the severe acute respiratory syndrome is SARS-associated coronavirus 2.

Embodiment 75. The method of Embodiment 71, wherein the severe acute respiratory syndrome is MERS-associated coronavirus.

Embodiment 76. A method of treating Middle Eastern respiratory syndrome in a subject in need thereof, the method comprising administering to the subject an effective amount of the nucleic acid of any one of Embodiments 1 to 61; the lipid nanoparticle of any one of Embodiments 62 to 64; or the pharmaceutical composition of any one of Embodiments 65 to 68.

Embodiment 77. The method of any one of Embodiments 69 to 76, comprising nasally administering.

Embodiment 78. The method of any one of Embodiments 69 to 76, comprising intravenously administering.

Embodiment 79. A drug delivery device comprising an effective amount of the nucleic acid of any one of Embodiments 1 to 61; the lipid nanoparticle of any one of Embodiments 62 to 64; or the pharmaceutical composition of any one of Embodiments 65 to 68.

Embodiment 80. The drug delivery device of Embodiment 79, wherein the drug delivery device is a nebulizer.

Embodiment 81. The drug delivery device of Embodiment 79, wherein the drug delivery device is a syringe.

Embodiment 82. The drug delivery device of Embodiment 79, wherein the drug delivery device is a ventilator.

Embodiment 83. A ventilator comprising the drug delivery device of any one of Embodiments 79 to 81.

Informal Sequence Listing

For SEQ ID NOS:1-8, “m” preceding a nucleotide means that nucleotide based is modified with 2′O-methyl, * refers to a phosphorothioate linkage between two nucleotides. The nucleotides of SEQ ID NOS:1-8 have ribose sugars except that those marked with “(end quotes) which have a deoxyribose sugar.

= UTR3 (unmodified dsiRNA sense sequence)
SEQ ID NO: 1
5′ AUACCUUCCCAGGUAACAAACCAA″C″ 3′
= UTR3 (dsiRNA sense sequence)
SEQ ID NO: 2
5′AmUmACmCmUUCCmCmAGGUAmAmCA* A*ACCAA″C″3′,
= siUTR3 (unmodified siRNA antisense sequence)
SEQ ID NO: 3
5′ GUUGGUUUGUUACCUGGGAAGGUAUAA 3′
= siUTR3 (siRNA antisense sequence)
SEQ ID NO: 4
5′ G*UUGGUUUGUmUmAmCCUGGGAAGGmUAmUmAA 3′
= siUTR1 (unmodified dsiRNA sense sequence)
SEQ ID NO: 5
5′GUCCCUGGUUUCAACGAGAAAACA″C″ 3′
= siUTR1 (dsiRNA sense sequence)
SEQ ID NO: 6
5′GmUmCCmCmUGGUUmUmCAmAmCGAG*A*AAAmCA″C″′ 3′
= siUTR1 (unmodified siRNA antisense sequence)
SEQ ID NO: 7
5′ GUGUUUUCUCGUUGAAACCAGGGACAA 3′
= siUTR1 (siRNA antisense sequence)
SEQ ID NO: 8
3′ AmAmCmAGGGmAmCCAAAGUUGmCmUCUUUUGU*G5′
SEQ ID NO: 9
5′CAUAUUGCGCGUAUAGUCGCGUUAG3′
SEQ ID NO: 10
5′CmAmUmAmUUGCGCGmUmAmUmAGUC*G*CGUmUA″G″′ 3′
SEQ ID NO: 11
3′ UGGUAUAACGCGCAUAUCAGCGCAAUC 5′
SEQ ID NO: 12
3′ UGGmUAmUmAACGCGCAmUmAmUmCAGCGCAAU*C 5′
SEQ ID NO: 13
5′-GTCCCTGGTTTCAACGAGAAAACAC
SEQ ID NO: 14
5′-GCTTAGTAGAAGTTGAAAAAGGCGT
SEQ ID NO: 15
5′-TGTCCCTGGTTTCAACGAGAAAACA
SEQ ID NO: 16
5′-ATACCTTCCCAGGTAACAAACCAAC
SEQ ID NO: 17
5′-CCCTGGTTTCAACGAGAAAACACAC
SEQ ID NO: 18
5′ AAGGTAAGATGGAGAGCCTTGTCCC
SEQ ID NO: 19
5′ AGGTAAGATGGAGAGCCTTGTCCCT
SEQ ID NO: 20
5′ GGTAAGATGGAGAGCCTTGTCCCTG
SEQ ID NO: 21
5′-GTTTACAGAATAAATTGGATCACCG
SEQ ID NO: 22
5′-CTGTTTACAGAATAAATTGGATCAC
SEQ ID NO: 23
5′-CTATTACCGTTGAAGAGCTTAAAAA
SEQ ID NO: 24
5′-GTACTATTACCGTTGAAGAGCTTAA
SEQ ID NO: 25
5′-GGATTGGCAACTATAAATTAAACAC

With respect to SEQ ID NOS:26-49, the underlined portion of the sequence represents the regions of the sequence targeted by the siRNA described herein.

SARS-COV-2-5′UTR = SEQ ID NOS: 26-49
SEQ ID NO: 26
ATACCTTCCCAGGTAACAAACCAAC
SEQ ID NO: 27
GTCCCTGGTTTCAACGAGAAAACACACG
SEQ ID NO: 28
AAGATGGCACTTGTGGCTTAGTAGAAGTTGAAAAAGGCGT
MN985325 2019-nCoVUSA-WA12020
SEQ ID NO: 29
1...ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
MN988668 2019-nCoV WHU01
SEQ ID NO: 30
1...
TTAAAGGTTTATACCTTCCCAGGTAACAAACCAACCAACTTTCGATCTCTTGTAGAT
CTGTTCTCTAAACGAACTTTAAAATCTGTGTGGCTGTCACTCGGCTGCATGCTTAGT
GCACTCACGCAGTATAATTAATAACTAATTACTGTCGTTGACAGGACACGAGTAAC
TCGTCTATCTTCTGCAGGCTGCTTACGGTTTCGTCCGTGTTGCAGCCGATCATCAGC
ACATCTAGGTTTCGTCCGGGTGTGACCGAAAGGTAAGATGGAGAGCCTTGTCCCTG
GTTTCAACGAGAAAACACACGTCCAACTCAGTTTGCCTGTTTTACAGGTTCGCGACG
TGCTCGTACGTGGCTTTGGAGACTCCGTGGAGGAGGTCTTATCAGAGGCACGTCAA
CATCTTAAAGATGGCACTTGTGGCTTAGTAGAAGTTGAAAAAGGCGTTTT...446
MN988669 2019-nCoV WHU02
SEQ ID NO: 31
1...
TTAAAGGTTTATACCTTCCCAGGTAACAAACCAACCAACTTTCGATCTCTTGTAGAT
CTGTTCTCTAAACGAACTTTAAAATCTGTGTGGCTGTCACTCGGCTGCATGCTTAGT
GCACTCACGCAGTATAATTAATAACTAATTACTGTCGTTGACAGGACACGAGTAAC
TCGTCTATCTTCTGCAGGCTGCTTACGGTTTCGTCCGTGTTGCAGCCGATCATCAGC
ACATCTAGGTTTCGTCCGGGTGTGACCGAAAGGTAAGATGGAGAGCCTTGTCCCTG
GTTTCAACGAGAAAACACACGTCCAACTCAGTTTGCCTGTTTTACAGGTTCGCGACG
TGCTCGTACGTGGCTTTGGAGACTCCGTGGAGGAGGTCTTATCAGAGGCACGTCAA
CATCTTAAAGATGGCACTTGTGGCTTAGTAGAAGTTGAAAAAGGCGTTTT...446
MT019532 BetaCoV Wuhan PBCAMS-WH-04 2019
SEQ ID NO: 32
1...ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
MT050493 SARS-COV-2 166 human 2020 IND
SEQ ID NO: 33
1...CAGGTAACAAACCAACCAACTTTCGATCTCTTGTAGATCTGTTCTCTAAACGAAC
TTTAAAATCTGTGTGGCTGTCACTCGGCTGCATGCTTAGTGCACTCACGCAGTATAA
TTAATAACTAATTACTGTCGTTGACAGGACACGAGTAACTCGTCTATCTTCTGCAGG
CTGCTTACGGTTTCGTCCGTGTTGCAGCCGATCATCAGCACATCTAGGTTTCGTCCG
GGTGTGACCGAAAGGTAAGATGGAGAGCCTTGTCCCTGGTTTCAACGAGAAAACAC
ACGTCCAACTCAGTTTGCCTGTTTTACAGGTTCGCGACGTGCTCGTACGTGGCTTTG
GAGACTCCGTGGAGGAGGTCTTATCAGAGGCACGTCAACATCTTAAAGATGGCACT
TGTGGCTTAGTAGAAGTTGAAAAAGGCGTTTT...427
MT066156 SARS-COV-2 INMI1 human 2020 ITA
SEQ ID NO: 34
1...ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
MT093571 SARS-COV-2 01 human 2020 SWE
SEQ ID NO: 35
1...ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
MT106054 2019-nCoV USA-TX1 2020
SEQ ID NO: 36
1...ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
MT118835 2019-nCoV USA-CA9 2020
SEQ ID NO: 37
1...ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
MT126808 SARS-COV-2 SP02 human 2020 BRA
SEQ ID NO: 38
1...ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
MT184913 2019-nCoV USA-CruiseA-26 2020
SEQ ID NO: 39
1...ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
MT188341 USA MN1-MDH1 2020
SEQ ID NO: 40
1...AGATCTGTTCTCTAAACGAACTTTAAAATCTGTGTGGCTGTCACTCGGCTGCATG
CTTAGTGCACTCACGCAGTATAATTAATAACTAATTACTGTCGTTGACAGGACACGA
GTAACTCGTCTATCTTCTGCAGGCTGCTTACGGTTTCGTCCGTGTTGCAGCCGATCAT
CAGCACATCTAGGTTTCGTCCGGGTGTGACCGAAAGGTAAGATGGAGAGCCTTGTC
CCTGGTTTCAACGAGAAAACACACGTCCAACTCAGTTTGCCTGTTTTACAGGTTCGC
GACGTGCTCGTACGTGGCTTTGGAGACTCCGTGGAGGAGGTCTTATCAGAGGCACG
TCAACATCTTAAAGATGGCACTTGTGGCTTAGTAGAAGTTGAAAAAGGCGTTTT...392
MT192759 SARS-COV-2 CGMH-CGU-01 human2020 TWN
SEQ ID NO: 41
1...CAAACCAACCAACTTTCGATCTCTTGTAGATCTGTTCTCTAAACGAACTTTAAAA
TCTGTGTGGCTGTCACTCGGCTGCATGCTTAGTGCACTCACGCAGTATAATTAATAA
CTAATTACTGTCGTTGACAGGACACGAGTAACTCGTCTATCTTCTGCAGGCTGCTTA
CGGTTTCGTCCGTGTTGCAGCCGATCATCAGCACATCTAGGTTTCGTCCGGGTGTGA
CCGAAAGGTAAGATGGAGAGCCTTGTCCCTGGTTTCAACGAGAAAACACACGTCCA
ACTCAGTTTGCCTGTTTT...420
MT192773 SARS-COV-2 nCoV-19-02S human 2020 VNM
SEQ ID NO: 42
1...ATTAAAGGTTTATACCTTCCCAGGTATAAACCAACCAACTTTCGATCTCTTGTAG
ATCTGTTCTCTAAACGAACTTTAAAATCTGTGTGGCTGTCACTCGGCTGCATGCTTA
GTGCACTCACGCAGTATAATTAATAACTAATTACTGTCGTTGACAGGACACGAGTA
ACTCGTCTATCTTCTGCAGGCTGCTTACGGTTTCGTCCGTGTTGCAGCCGATCATCA
GCACATCTAGGTTTCGTCCGGGTGTGACCGAAAGGTAAGATGGAGAGCCTTGTCCC
TGGTTTCAACGAGAAAACACACGTCCAACTCAGTTTGCCTGTTTTACAGGTTCGCGA
CGTGCTCGTACGTGGCTTTGGAGACTCCGTGGAGGAGGTCTTATCAGAGGCACGTC
AACATCTTAAAGATGGCACTTGTGGCTTAGTAGAAGTTGAAAAAGGCGTTTT...446
NC_045512 Wuhan-Hu-1
SEQ ID NO: 43
1...ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
MT066176 SARS-COV-2 NTU02 2020 TWN
SEQ ID NO: 44
1...ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
MT049951 SARS-COV-2 Yunnan-01 human 2020 CHN
SEQ ID NO: 45
1...ATTAAAGGTTTATACCTTCCCAGGTAACAAACCAACCAACTTTCGATCTCTTGTA
GATCTGTTCTCTAAACGAAATTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
MN988713 2019-NCOV USA-IL1 2020
SEQ ID NO: 46
1...ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
MT019533 BETACOV WUHAN IPBCAMS-WH-05 2020
SEQ ID NO: 47
1...ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
MT039873 SARS-COV-2 HZ-1
SEQ ID NO: 48
1...AAAGGTTTATACCTTCCCAGGTAACAAACCAACCAACTTTCGATCTCTTGTAGAT
CTGTTCTCTAAACGAACTTTAAAATCTGTGTGGCTGTCACTCGGCTGCATGCTTAGT
GCACTCACGCAGTATAATTAATAACTAATTACTGTCGTTGACAGGACACGAGTAAC
TCGTCTATCTTCTGCAGGCTGCTTACGGTTTCGTCCGTGTTGCAGCCGATCATCAGC
ACATCTAGGTTTCGTCCGGGTGTGACCGAAAGGTAAGATGGAGAGCCTTGTCCCTG
GTTTCAACGAGAAAACACACGTCCAACTCAGTTTGCCTGTTTTACAGGTTCGCGACG
TGCTCGTACGTGGCTTTGGAGACTCCGTGGAGGAGGTCTTATCAGAGGCACGTCAA
CATCTTAAAGATGGCACTTGTGGCTTAGTAGAAGTTGAAAAAGGCGTTTT...444
MT039890 SARS-COV-2 SNU01
SEQ ID NO: 49
1...ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGATGGAGAGCCTTGTCC
CTGGTTTCAACGAGAAAACACACGTCCAACTCAGTTTGCCTGTTTTACAGGTTCGCG
ACGTGCTCGTACGTGGCTTTGGAGACTCCGTGGAGGAGGTCTTATCAGAGGCACGT
CAACATCTTAAAGATGGCACTTGTGGCTTAGTAGAAGTTGAAAAAGGCGTTTT...447
SARS-COV-1 5′UTR
NC_004718 SARS COV 1
SEQ ID NO: 50
1...ATATTAGGTTTTTACCTACCCAGGAAAAGCCAACCAACCTCGATCTCTTGTAGAT
CTGTTCTCTAAACGAACTTTAAAATCTGTGTAGCTGTCGCTCGGCTGCATGCCTAGT
GCACCTACGCAGTATAAACAATAATAAATTTTACTGTCGTTGACAAGAAACGAGTA
ACTCGTCCCTCTTCTGCAGACTGCTTACGGTTTCGTCCGTGTTGCAGTCGATCATCAG
CATACCTAGGTTTCGTCCGGGTGTGACCGAAAGGTAAGATGGAGAGCCTTGTTCTTG
GTGTCAACGAGAAAACACACGTCCAACTCAGTTTGCCTGTCCTTCAGGTTAGAGAC
GTGCTAGTGCGTGGCTTCGGGGACTCTGTGGAAGAGGCCCTATCGGAGGCACGTGA
ACACCTCAAAAATGGCACTTGTGGTCTAGTAGAGCTGGAAAAAGGCGTACT...447
SEQ ID NO: 51
TTACCTACCCAGGAAAAGCCAAC
SEQ ID NO: 52
TGTTCTTGGTGTCAACGAGAAAACACAC
SEQ ID NO: 53
AAAATGGCACTTGTGGTCTAGTAGAGCTGGAAAAAGGCGT
SARS-COV-2 (COVID-19) M ORF = SEQ ID NOS: 54-77
SEQ ID NO: 54
ACGGTACTATTACCGTTGAAGAGCTTAAAA
SEQ ID NO: 55
CTGTTTACAGAATAAATTGGATCACCG
SEQ ID NO: 56
GGATTGGCAACTATAAATTAAACAC
MN985325 2019-nCoVUSA-WA12020
SEQ ID NO: 57
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MN988668 2019-nCoV WHU01
SEQ ID NO: 58
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MN988669 2019-nCoV WHU02
SEQ ID NO: 59
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT019532 BetaCoV Wuhan PBCAMS-WH-04 2019
SEQ ID NO: 60
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT050493 SARS-COV-2 166 human 2020 IND
SEQ ID NO: 61
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT066156 SARS-COV-2 INMI1 human 2020 ITA
SEQ ID NO: 62
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT093571 SARS-COV-2 01 human 2020 SWE
SEQ ID NO: 63
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT106054 2019-nCoV USA-TX1 2020
SEQ ID NO: 64
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT118835 2019-nCoV USA-CA9 2020
SEQ ID NO: 65
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT126808 SARS-COV-2 SP02 human 2020 BRA
SEQ ID NO: 66
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT184913 2019-nCoV USA-CruiseA-26 2020
SEQ ID NO: 67
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT188341 USA MN1-MDH1 2020
SEQ ID NO: 68
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT192759 SARS-COV-2 CGMH-CGU-01 human2020 TWN
SEQ ID NO: 69
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT192773 SARS-COV-2 nCoV-19-02S human 2020 VNM
SEQ ID NO: 70
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
NC_045512 Wuhan-Hu-1
SEQ ID NO: 71
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT066176 SARS-COV-2 NTU02 2020 TWN
SEQ ID NO: 72
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT049951 SARS-COV-2 YUNNAN-01 HUMAN 2020 CHN
SEQ ID NO: 73
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MN988713 2019-NCOV USA-IL1 2020
SEQ ID NO: 74
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT019533 BETACOV WUHAN IPBCAMS-WH-05 2020
SEQ ID NO: 75
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
MT039873 SARS-COV-2 HZ-1
SEQ ID NO: 76
26519...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27189
MT039890 SARS-COV-2 SNU01
SEQ ID NO: 77
26552...ATGGCAGATTCCAACGGTACTATTACCGTTGAAGAGCTTAAAAAGCTCCTT
GAACAATGGAACCTAGTAATAGGTTTCCTATTCCTTACATGGATTTGTCTTCTACAA
TTTGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAATTG
GATCACCGGTGGAATTGCTATCGCAATGGCTTGTCTTGTAGGCTTGATGTGGCTCAG
CTACTTCATTGCTTCTTTCAGACTGTTTGCGCGTACGCGTTCCATGTGGTCATTCAAT
CCAGAAACTAACATTCTTCTCAACGTGCCACTCCATGGCACTATTCTGACCAGACCG
CTTCTAGAAAGTGAACTCGTAATCGGAGCTGTGATCCTTCGTGGACATCTTCGTATT
GCTGGACACCATCTAGGACGCTGTGACATCAAGGACCTGCCTAAAGAAATCACTGT
TGCTACATCACGAACGCTTTCTTATTACAAATTGGGAGCTTCGCAGCGTGTAGCAGG
TGACTCAGGTTTTGCTGCATACAGTCGCTACAGGATTGGCAACTATAAATTAAACAC
AGACCATTCCAGTAGCAGTGACAATATTGCTTTGCTTGTACAGTAA...27191
SARS-COV-1 M ORF = SEQ ID NO: 78-81
NC_004718 SARS COV 1
SEQ ID NO: 78
26397...ATGGCAGACAACGGTACTATTACCGTTGAGGAGCTTAAACAACTCCTGGAA
CAATGGAACCTAGTAATAGGTTTCCTATTCCTAGCCTGGATTATGTTACTACAATTT
GCCTATTCTAATCGGAACAGGTTTTTGTACATAATAAAGCTTGTTTTCCTCTGGCTCT
TGTGGCCAGTAACACTTGCTTGTTTTGTGCTTGCTGCTGTCTACAGAATTAATTGGGT
GACTGGCGGGATTGCGATTGCAATGGCTTGTATTGTAGGCTTGATGTGGCTTAGCTA
CTTCGTTGCTTCCTTCAGGCTGTTTGCTCGTACCCGCTCAATGTGGTCATTCAACCCA
GAAACAAACATTCTTCTCAATGTGCCTCTCCGGGGGACAATTGTGACCAGACCGCTC
ATGGAAAGTGAACTTGTCATTGGTGCTGTGATCATTCGTGGTCACTTGCGAATGGCC
GGACACTCCCTAGGGCGCTGTGACATTAAGGACCTGCCAAAAGAGATCACTGTGGC
TACATCACGAACGCTTTCTTATTACAAATTAGGAGCGTCGCAGCGTGTAGGCACTGA
TTCAGGTTTTGCTGCATACAACCGCTACCGTATTGGAAACTATAAATTAAATACAGA
CCACGCCGGTAGCAACGACAATATTGCTTTGCTAGTACAGTAA...27063
SEQ ID NO: 79
GTACTATTACCGTTGAGGAGCTTAA
SEQ ID NO: 80
GCTGTCTACAGAATTAATTGGGTGACTG
SEQ ID NO: 81
GTATTGGAAACTATAAATTAAATAC

Therapeutic Nucleic Acids. For SEQ ID NOS:82-91, each nucleotide is ribose except for those marked with” (end quotes) which are deoxy ribose.

= siUTR2 (dsiRNA sense sequence)
SEQ ID NO: 82
5′ GCUUAGUAGAAGUUGAAAAAGGCG″T″ 3′
= siUTR2 (siRNA antisense sequence)
SEQ ID NO: 83
5′ ACGCCUUUUUCAACUUCUACUAAGCCA″ 3′
= siUTR4 (dsiRNA sense sequence)
SEQ ID NO: 84
5′ AAGAUGGCACUUGUGGCUUAGUAG″A″ 3′
= siUTR4 (siRNA antisense sequence)
SEQ ID NO: 85
5′ UCUACUAAGCCACAAGUGCCAUCUUUA 3′
= siUTR5 (dsiRNA sense sequence)
SEQ ID NO: 86
5′ CCCUGGUUUCAACGAGAAAACACA″C″ 3′
= siUTR5 (siRNA antisense sequence)
SEQ ID NO: 87
5′ GUGUGUUUUCUCGUUGAAACCAGGGAC 3′
= siEnv1 (dsiRNA sense sequence)
SEQ ID NO: 88
5′ GUUUACAGAAUAAAUUGGAUCACC″G″ 3′
= siEnv1 (siRNA antisense sequence)
SEQ ID NO: 89
5′ CGGUGAUCCAAUUUAUUCUGUAAACAG 3′
= siEnv2 (dsiRNA sense sequence)
SEQ ID NO: 90
5′ CUGUUUACAGAAUAAAUUGGAUCA″C″ 3′
= siEnv2 (siRNA antisense sequence)
SEQ ID NO: 91
5′ GUGAUCCAAUUUAUUCUGUAAACAGCA 3′
= siEnv3 (dsiRNA sense sequence)
SEQ ID NO: 92
5′ CUAUUACCGUUGAAGAGCUUAAAA″A″ 3′
= siEnv3 (siRNA antisense sequence)
SEQ ID NO: 93
5′ UUUUUAAGCUCUUCAACGGUAAUAGUA 3′
= siEnv4 (dsiRNA sense sequence)
SEQ ID NO: 94
5′ GUACUAUUACCGUUGAAGAGCUUA″A″ 3′
= siEnv4 (siRNA antisense sequence)
SEQ ID NO: 95
5′ UUAAGCUCUUCAACGGUAAUAGUACCG 3′
= siEnv5 (dsiRNA sense sequence)
SEQ ID NO: 96
5′ GGAUUGGCAACUAUAAAUUAAACA″C″ 3′
= siEnv5 (siRNA antisense sequence)
SEQ ID NO: 97
5′ GUGUUUAAUUUAUAGUUGCCAAUCCUG 3
GenBank: MN985325.1
SEQ ID NO: 98
TTAAAGGTTTATACCTTCCCAGGTAACAAACCAACCAACTTTCGATCTCTTGTAGAT
CTGTTCTCTAAACGAACTTTAAAATCTGTGTGGCTGTCACTCGGCTGCATGCTTAGT
GCACTCACGCAGTATAATTAATAACTAATTACTGTCGTTGACAGGACACGAGTAAC
TCGTCTATCTTCTGCAGGCTGCTTACGGTTTCGTCCGTGTTGCAGCCGATCATCAGC
ACATCTAGGTTTCGTCCGGGTGTGACCGAAAGGTAAGATGGAGAGCCTTGTCCCTG
GTTTCAACGAGAAAACACACGTCCAACTCAGTTTGCCTGTTTTACAGGTTCGCGACG
TGCTCGTACGTGGCTTTGGAGACTCCGTGGAGGAGGTCTTATCAGAGGCACGTCAA
CATCTTAAAGATGGCACTTGTGGCTTAGTAGAAGTTGAAAAAGGCGTTTTGCCTCAA
CTTGAACAGCCCTATGTGTTC
SEQ ID NO: 99
ATGGCAGATTCCAACGGTACTATTACCGTTGAAGAGCTTAAAAAGCTCCTTGAACA
ATGGAACCTAGTAATAGGTTTCCTATTCCTTACATGGATTTGTCTTCTACAATTTGCC
TATGCCAACAGGAATAGGTTTTTGTATATAATTAAGTTAATTTTCCTCTGGCTGTTAT
GGCCAGTAACTTTAGCTTGTTTTGTGCTTGCTGCTGTTTACAGAATAAATTGGATCA
CCGGTGGAATTGCTATCGCAATGGCTTGTCTTGTAGGCTTGATGTGGCTCAGCTACT
TCATTGCTTCTTTCAGACTGTTTGCGCGTACGCGTTCCATGTGGTCATTCAATCCAGA
AACTAACATTCTTCTCAACGTGCCACTCCATGGCACTATTCTGACCAGACCGCTTCT
AGAAAGTGAACTCGTAATCGGAGCTGTGATCCTTCGTGGACATCTTCGTATTGCTGG
ACACCATCTAGGACGCTGTGACATCAAGGACCTGCCTAAAGAAATCACTGTTGCTA
CATCACGAACGCTTTCTTATTACAAATTGGGAGCTTCGCAGCGTGTAGCAGGTGACT
CAGGTTTTGCTGCATACAGTCGCTACAGGATTGGCAACTATAAATTAAACACAGAC
CATTCCAGTAGCAGTGACAATATTGCTTTGCTTGTACAGTAA
= eGFP as400
SEQ ID NO: 222
tgccgtcctccttgaagtcgatgcccttcagctcgatgcggttcaccagggtgtcgccctcgaacttcacctcggcgcgggtcttgtagttgc
cgtcgtccttgaagaagatggtgcgctcctggacgtagccttcgggcatggcggacttgaagaagtcgtgctgcttcatgtggtcggggta
gcggctgaagcactgcacgccgtaggtcagggtggtcacgagggtgggccagggcacgggcagcttgccggtggtgcagatgaacttc
agggtcagcttgccgtaggtggcatcgccctcgccctcgccggacacgctgaacttgtggccgtttacgtcgccgtccagctcgaccagg
atgggcaccaccccggtgaacagctcctcgcccttgct
= N as600
SEQ ID NO: 266
cttgtctgattagttcctggtccccaaaatttccttgggtttgttctggaccacgtctgccgaaagcttgtgttacattgtatgctttagtggcag
tacgtttttgccgaggcttcttagaagcctcagcagcagatttcttagtgacagtttggccttgttgttgttggcctttaccagacattttgctct
caagctggttcaatctgtcaagcagcagcaaagcaagagcagcatcaccgccattgccagccattctagcaggagaagttcccctactgctgcct
ggagttgaatttcttgaactgttgcgactacgtgatgaggaacgagaagaggcttgactgccgcctctgctcccttctgcgtagaagccttttg
gcaatgttgttccttgaggaagttgtagcacgattgcagcattgttagcaggattgcgggtgccaatgtgatcttttggtgtattcaaggctccc
tcagttgcaacccatatgatgccgtctttgttagcaccatagggaagtccagcttctggcccagttcctaggtagtagaaataccatcttggac
tgagatctttcattttaccgtcaccaccac
= N as800
SEQ ID NO: 267
tttgtatgcgtcaatatgcttattcagcaaaatgacttgatctttgaaatttggatctttgtcatccaatttgatggcacctgtgtaggtcaacca
cgttcccgaaggtgtgacttccatgccaatgcgcgacattccgaagaacgctgaagcgctgggggcaaattgtgcaatttgcggccaatgtttg
taatcagttccttgtctgattagttcctggtccccaaaatttccttgggtttgttctggaccacgtctgccgaaagcttgtgttacattgtatgct
ttagtggcagtacgtttttgccgaggcttcttagaagcctcagcagcagatttcttagtgacagtttggccttgttgttgttggcctttaccagac
attttgctctcaagctggttcaatctgtcaagcagcagcaaagcaagagcagcatcaccgccattgccagccattctagcaggagaagttcccct
actgctgcctggagttgaatttcttgaactgttgcgactacgtgatgaggaacgagaagaggcttgactgccgcctctgctcccttctgcgta
gaagccttttggcaatgttgttccttgaggaagttgtagcacgattgcagcattgttagcaggattgcgggtgccaatgtgatcttttggtgtat
tcaaggctccctcagttgcaacccatatgatgccgtctttgttagcaccatagggaagtccagcttctggcccagttcctaggtagtagaaata
ccatcttggactgagatctttcattttaccgtcaccaccac
= RdRp 5′ as400
SEQ ID NO: 268
tctgccattgtgtatttagtaagacgttgacgtgatatatgtggtaccatgtcaccgtctattctaaacttaaagaagtcatgtttagcaacagct
ggacaatccttaagtaaattataaattgtttcttcatgttggtagttagagaaagtgtgtctcttaactacaaagtaagaatcaattaaattgtca
tcttcgtccttttcttggaagcgacaacaattagtttttaggaatttagcaaaaccagctactttatcattgtagatgtcaaaagccctgtatacg
acatcagtactagtgcctgtgccgcacggtgtaagacgggctgcacttacaccgcaaacccgtttaaaaacgattgtgcatcagctgactgaagc
atgggttcgcggagttg
= RdRp 5′ as600
SEQ ID NO: 269
agaattgtactgtttttaacaaagcttggcgtacacgttcacctaagttggcgtatacgcgtaatatatctgggttttctacaaaatcataccagt
cctttttattgaaataatcatcatcacaacaattgtatgtgacaagtatttcttttaatgtgtcacaattaccttcatcaaaatgccttaaagcat
agacgaggtctgccattgtgtatttagtaagacgttgacgtgatatatgtggtaccatgtcaccgtctattctaaacttaaagaagtcatgtttag
caacagctggacaatccttaagtaaattataaattgtttcttcatgttggtagttagagaaagtgtgtctcttaactacaaagtaagaatcaatta
aattgtcatcttcgtccttttcttggaagcgacaacaattagtttttaggaatttagcaaaaccagctactttatcattgtagatgtcaaaagccc
tgtatacgacatcagtactagtgcctgtgccgcacggtgtaagacgggctgcacttacaccgcaaacccgtttaaaaacgattgtgcatcagctga
ctgaagcatgggttcgcggagttg
= RdRp 5′ as800
SEQ ID NO: 270
agtgtcaacatgtgactctgcagttaaagccctggtcaaggttaatataggcattaacaatgaataataagaatctacaacaggaactccact
acctggcgtggtttgtatgaaatcaccgaaatcataccagttaccattgagatcttgattatctaatgtcagtacaccaacaataccagcatttc
gcatggcatcacagaattgtactgtttttaacaaagcttggcgtacacgttcacctaagttggcgtatacgcgtaatatatctgggttttctacaa
aatcataccagtcctttttattgaaataatcatcatcacaacaattgtatgtgacaagtatttcttttaatgtgtcacaattaccttcatcaaaat
gccttaaagcatagacgaggtctgccattgtgtatttagtaagacgttgacgtgatatatgtggtaccatgtcaccgtctattctaaacttaaaga
agtcatgtttagcaacagctggacaatccttaagtaaattataaattgtttcttcatgttggtagttagagaaagtgtgtctcttaactacaaagt
aagaatcaattaaattgtcatcttcgtccttttcttggaagcgacaacaattagtttttaggaatttagcaaaaccagctactttatcattgtaga
tgtcaaaagccctgtatacgacatcagtactagtgcctgtgccgcacggtgtaagacgggctgcacttacaccgcaaacccgtttaaaaacgattg
tgcatcagctgactgaagcatgggttcgcggagttg
= RdRp Cat as400 (or RdRp Cat 400)
SEQ ID NO: 271
taaagaactgacttaaagttctttatgctagccactagaccttgagatgcataagtgctattgaaacacacaacagcatcgtcagagagtatca
tcattgagaaatgtttacgcaaatatgcgtaaaactcattcacaaagtctgtgtcaacatctctatttctatagagacactcataaagtctgtgt
tgtaaattgcggacatacttatcggcaattttgttaccatcagtagataaaagtgcattaacattggccgtgacagcttgacaaatgttaaaaac
actattagcataagcagttgtggcatctcctgatgaggttccacctggtttaacatatagtgaaccgccacacatgaccatttcactcaatactt
gagcacactcattagctaatc
= RdRp Cat as600 (or RdRp Cat 600)
SEQ ID NO: 272
taaagaactgacttaaagttctttatgctagccactagaccttgagatgcataagtgctattgaaacacacaacagcatcgtcagagagtatca
tcattgagaaatgtttacgcaaatatgcgtaaaactcattcacaaagtctgtgtcaacatctctatttctatagagacactcataaagtctgtgt
tgtaaattgcggacatacttatcggcaattttgttaccatcagtagataaaagtgcattaacattggccgtgacagcttgacaaatgttaaaaac
actattagcataagcagttgtggcatctcctgatgaggttccacctggtttaacatatagtgaaccgccacacatgaccatttcactcaatactt
gagcacactcattagctaatctatagaaacggtgtgacaagctacaacacgttgtatgtttgcgagcaagaacaagtgaggccataattctaagc
atgttaggcatggctctatcacatttaggataatcccaacccataaggtgagggttttctacatcactataaacagtttttaacatgttgtgccaa
ccaccatagaatttgcttgttccaattact
= RdRp Cat as800 (or RdRp Cat 800)
SEQ ID NO: 273
taaagaactgacttaaagttctttatgctagccactagaccttgagatgcataagtgctattgaaacacacaacagcatcgtcagagagtatca
tcattgagaaatgtttacgcaaatatgcgtaaaactcattcacaaagtctgtgtcaacatctctatttctatagagacactcataaagtctgtgt
tgtaaattgcggacatacttatcggcaattttgttaccatcagtagataaaagtgcattaacattggccgtgacagcttgacaaatgttaaaaac
actattagcataagcagttgtggcatctcctgatgaggttccacctggtttaacatatagtgaaccgccacacatgaccatttcactcaatactt
gagcacactcattagctaatctatagaaacggtgtgacaagctacaacacgttgtatgtttgcgagcaagaacaagtgaggccataattctaagc
atgttaggcatggctctatcacatttaggataatcccaacccataaggtgagggttttctacatcactataaacagtttttaacatgttgtgccaa
ccaccatagaatttgcttgttccaattactacagtagctcctctagtggcggctattgatttcaataatttttgatgaaactgtctattggtcata
gtactacagatagagacaccagctacggtgcgagctctattctttgcactaatggcatacttaagattcatttgagttatagtagggatgacatta
cgttttgtatatgcgaaaagtgcatcttgatcctcata

### EXAMPLES

The following examples are for purposes of illustration only and are not intended to limit the spirit or scope of the disclosure or claims.

The disclosure provides an RNA interference approach that utilizes small interfering RNAs (siRNAs) targeted to SARS-CoV-2. See Casucci et al, Front Immunol., 9:507 (2018). Coronaviruses are RNA viruses which have been found to be highly-susceptible to RNAi. See Wu et; al, Antiviral Res., 65(1):45-8 (2005); and Shi et al, Cell Res., 15(3):193-200 (2005). RNAi is a relatively potent facile method that can target and repress gene and viral transcripts in a targeted manner and one RNAi treatment (patisiran) has recently been approved by the FDA to treat high-cholesterol. Patisiran is a chemically stabilized siRNA that is delivered to the liver using lipid nanoparticle formulations (LNP). LNP formulations have a predilection for targeting the liver and one issue that plagues RNAi therapeutics, in the context of viral infections, is the delivery of the therapeutic siRNAs to the viral infected cells.

### Example 1

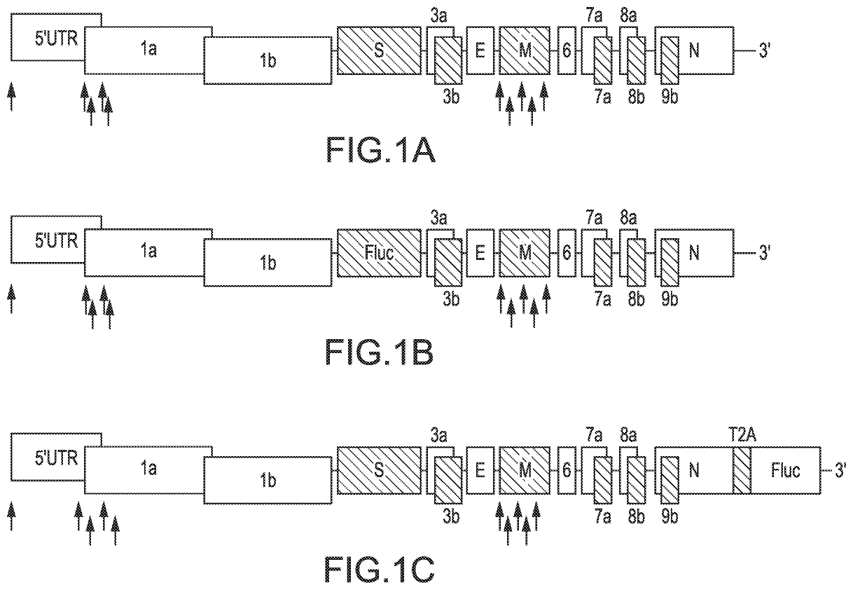

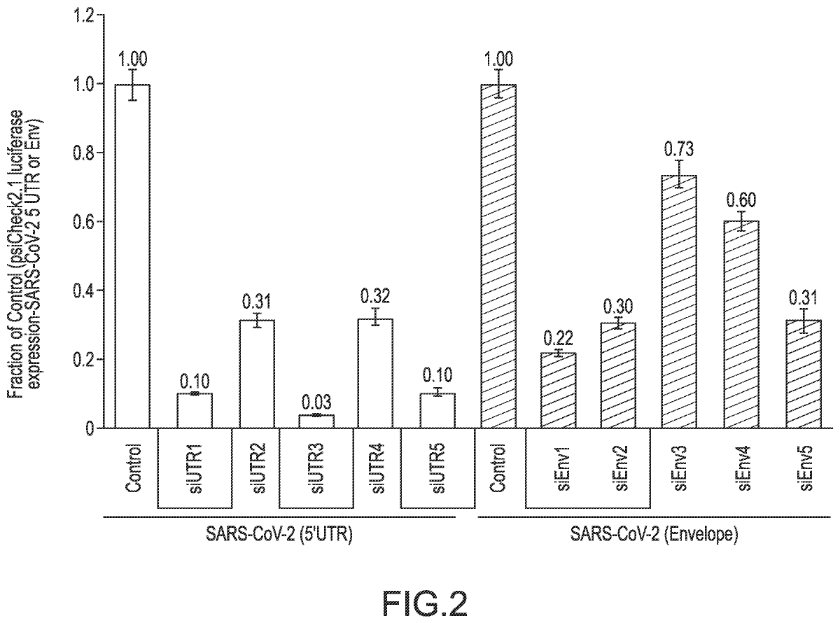

Towards this goal of treating SARS-CoV-2, a screen of siRNAs targeted to the 5′UTR or essential membrane protein (M) of SARS-CoV-2 (FIGS. 1A-1B) was carried out using the luciferase system knockdown assay to determine the top candidate siRNAs for targeting SARS-CoV-2; as described in Barichievy et al, Oligonucleotides, 17(4):419-31 (2007); and Weinberg et al, Nucleic acids research, 35(21):7303-12 (2007). Several candidate siRNAs were found to target the 5′ UTR or protein M RNAs of SARS-CoV-2 (FIG. 2). The sense target strand RNA in SARS-CoV-2 are shown in Tables F and G.

As shown in FIG. 2, the top-candidate targeted siRNAs were siUTR3, siUTR1, siEnv1 and siEnv5. These data indicate that the siRNA UTR1, 3 and 5 target site in the 5′ UTR is a conserved motif, as multiple siRNAs are functional at this site and that siUTR3 is the most repressive siRNA in the 5′ UTR and siEnv1 in the protein M.

### Example 2

In light of these data shown in Example 1, the inventors developed therapeutic siRNAs for treating COVID-19. The inventors developed and tested siRNA with various in vivo stabilizing chemical modifications. Chemical modifications protect the siRNAs from degradation and limit off target immune responses to the siRNAs. The formulated siUTR3 have been developed and are being tested for efficacy, stability, and deliverability via LNP. More particularly, the siRNAs shown in Tables F and G targeted to SEQ ID NO:98 (5′UTR of MN985325 2019-nCoVUSA-WA12020) or SEQ ID NO:99 (essential protein M of MN985325 2019-nCoVUSA-WA12020) will be screened against the psi-check system, which will be prepared with SEQ ID NO:98 (5′UTR of MN985325 2019-nCoVUSA-WA12020) or SEQ ID NO:99 (essential protein M of MN985325 2019-nCoVUSA-WA12020). The top 3 will be selected and then cloned into both the pre-miR-451 microRNA backbone (Reshke et al, Nature Biomedical Engineering, 4:52-68 (January 2020)) and the impending polycistronic CD containing miRNA expression system for exosome packaging and screened against virus infected cells by direct transfection and exposure to siRNA containing exosomes.

### Example 3

In light of these data resulting from Examples 1 and 2, the siRNA will be combined with an aerosolized lipid nanoparticle (LNP) or intranasal formulated LNP and test the ability to deliver the formulations to SARS-CoV-2 infected lung tissues in mice. The dosage needed to use this formulation with a nebulizer or intranasal administration in humans will be determined, and any inherent off-target issues that may arise from the single and cocktails of siRNAs. In particular, the efficacy, dosage and toxicology of intranasal and nebulized LNP-siRNA formulations will be determined in vivo using: (a) D5W transfected mice, described in Tang et al, Methods Mol Biol, 442:139-158 (2008), and (b) the K18-hACE2 mouse model (McCray et al, J. Virol, 81(2):813-821 (2007) infected with pseudotyped SARS-CoV-2 virus.

### Example 4

Several siRNAs appeared to repress reporter expression, with one candidate showing potent repression, siUTR3. Notably, siUTR3 targets 5′ UTR stem loop (SL1) a conserved and important viral element involved in transcription of SARS-CoV-2 (3, 14). These data support earlier findings that SARS-CoV-2, like other coronaviruses (8, 9), is also susceptible to RNAi. However, in the viral setting, early promising laboratory studies, such as demonstrating potent repression of virus in tissue culture have not translated into clinical success (15). The major issue is poor in vivo delivery. While naked siRNA was tried in vivo it was limited by rapid degradation and poor cellular uptake (16). Since then a range of delivery systems have emerged to allow prolonged circulation, resistance to serum nucleases, high accessibility to cells, efficient uptake and release of siRNA. These include nanoparticles (liposomes, dendrimers, viruses), condensed siRNA (polyethyleneimine PEI, chitosan), stabilized-naked siRNA chemistries, and tagged approaches (cholesterol, cell penetrating peptides) (17). The most clinically advanced system is liposome and is the essence of patisiran (ONPATTRO® by Alnylam Pharmaceuticals), but this formulation is for delivery to the liver.

Developing therapeutic strategies for viral infections based on siRNA has so far proved challenging, with poor clinical success primarily the result of poor delivery. New approaches are therefore required. Here we will investigate two novel nanoparticle approaches to deliver siRNAs (e.g., FIG. 2; Tables 1 and 2) targeted to SARS-CoV-2 infected cells in vivo. SARS-CoV-2 infection occurs predominantly in epithelial cells of the respiratory tract and results in diffuse alveolar damage (18). We propose to target these virus infected cells using an intravenous stealth liposome delivery platform (i.e., lipid nanoparticles as described herein); developed by our groups in (6, 19), and to contrast this intravenous approach with intranasal route of delivery of liposome containing siRNAs to treat SARS-CoV-2 infection in vivo.

Unlike standard liposomes, stealth lipid nanoparticles (LNPs) are formulated to be stable in serum, circulate for long periods of time, and to protect siRNA payloads from nucleases. These liposomes can be formulated, based on alterations of size, to traffic to the lung (6). SARS-CoV-2 is primarily a respiratory tract pathogen and infection in the lungs can become mucus-filled and inflamed, that may create a barrier that limits the ability of any nebulized LNP delivery approaches to result in poor siRNA penetration. Our hypothesis is that intravenous (IV) delivery of siRNA-loaded liposomes will deliver anti-SARS-CoV-2 siRNA payloads directly to the lung and bypass the mucosal issues, and this is the rational to simultaneously develop and assess the intranasal (IN) and IV stealth LNP approaches of delivery of the top-candidate SARS-CoV-2 siRNAs (e.g., FIG. 2, Tables 1 and 2).

The nucleic acids described herein will be in a lipid nanoparticle formulation comprising N-(1-(2,3-dioleoyloxy)propyl)-N,N,N-trimethylammonium chloride (DOTAP), cholesterol, dioleoylphosphatidylethanolamine (DOPE), and polyethylene glycol (PEG) having a molecular weight of about 2,000 conjugated to C16 ceramide. The lipid nanoparticle formulation may further comprise an isotonic sucrose solution. The lipid nanoparticles will have a size of about 125 nm+/−10%, a polydispersity index of about 0.3, a zeta potential of about 52 mM, and an siRNA entrapment efficiency of about 95%. See, e.g., McCaskill et al, Molecular Therapy-Nucleic Acids (2013) 2, e96; Wu et al, Pharm Res (2009) 26(3):512-522.

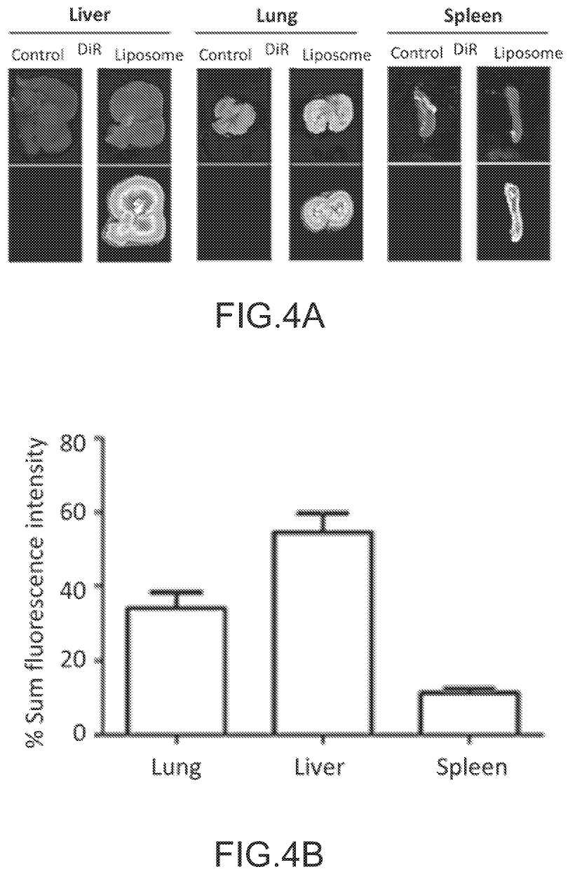

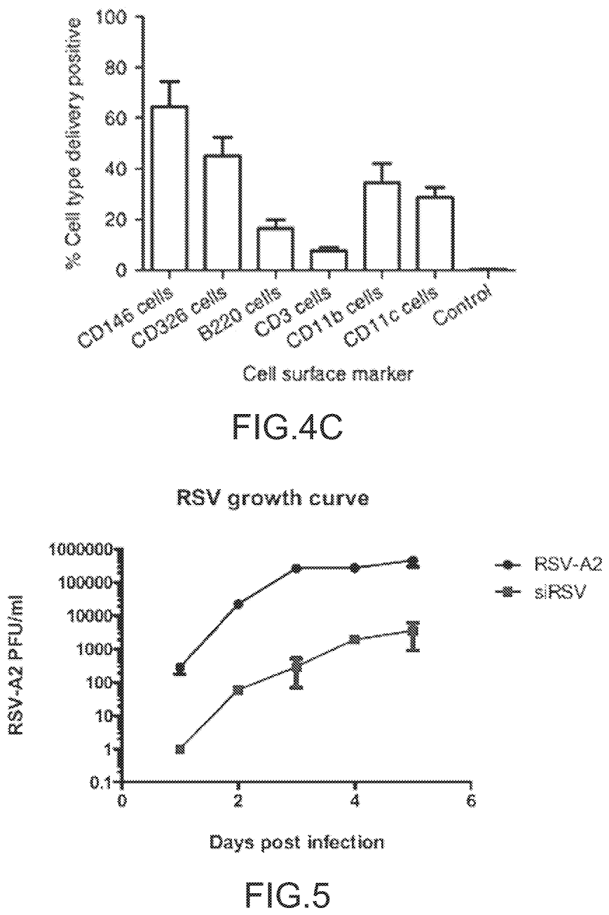

To determine the ability of stealth LNPs to effectively target the lung, DiR labelled LNPs were injected IV into mice and assessed 48 hours later. Robust DiR expression was observed in the lung, liver and less so in the spleen (FIGS. 4A-4B). Interestingly, CD146 and CD326, both markers of lung endothelial cells (20), demonstrated the greatest uptake of the stealth LNPs (FIG. 4C). These data indicate that stealth LNPs travel to and target the lung, as well as the liver, and less so to the spleen.

Next, we wanted to determine if these stealth LNPs could deliver siRNA payloads to repress a respiratory virus in the lung. Briefly, IV delivery results in about 35% of the payload in the lungs (FIGS. 4A-4B) where they transfect a range of cell types (FIG. 4C) including epithelial, endothelial and a range of immune cells. Using this system loaded with siRNAs targeting respiratory syncytial virus (RSV), a common and very contagious virus that infects the respiratory tract of children similar to SARS-CoV-2, there was a reduction in viral load of 2.5 logs (99%) (FIG. 5). These data indicate that stealth LNPs can deliver functional siRNAs to the lung to repress a highly contagious respiratory virus. SARS-CoV-2 infection occurs predominantly in epithelial cells of the respiratory tract and results in diffuse alveolar damage (18). We propose to target SARS-CoV-2 virus infected cells using intravenous stealth liposome delivery platform developed by our groups (6, 19) for delivering siRNA to treat SARS-CoV-2 infection (FIG. 2, Tables 1 and 2) in vivo.

Collectively, these preliminary studies indicate the following: (1) That we can generate, and test siRNAs targeted to SARS-CoV-2. (2) We contain sufficient expertise to generate I.V. injectable stealth LNPs and the ability to test these LNP formulations in vivo. According, we propose 3 AIMS to demonstrate that SARS-CoV-2 RNAi containing stealth LNPs can provide robust inhibition of viral expression in vivo.

AIM 1: Screen single and multiple siRNA targeting of SARS-CoV-2. Conserved regulatory regions in SARS-CoV-2 will be targeted with siRNAs and the efficacy and biological stability of chemically modified top candidate siRNAs determined in reporter single round virus infected cells. The top candidate siRNAs will be selected, and any off-target effects determined.

A.1.1. Rationale. A therapeutic compound that can specifically target and inhibit SARS-COV-2 in a systemic and potent manner that could be easily delivered to target the lungs in infected individuals to ameliorate COVID-19 disease would prove transformative. Those studies proposed in AIM 1 will develop and test candidate siRNAs and combinations of siRNAs with particular chemical modifications targeted to SARS-CoV-2 for efficacy, potency, off-target effects and deliverability in various LNP formulations.

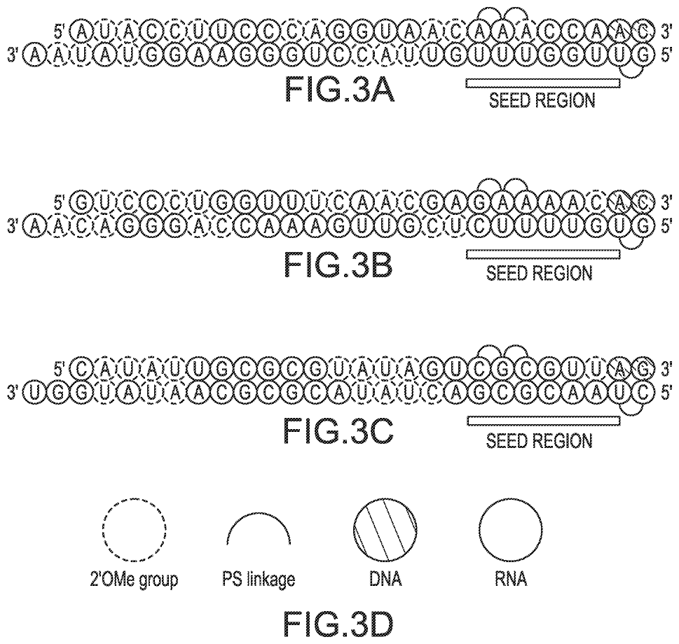

A.1.2. Generation and testing of SARS-CoV-2 targeted RNAi. A screen of siRNAs targeted to the 5′UTR or M of SARS-CoV-2 (e.g., FIG. 1, Tables 1 and 2) was carried out using the luciferase system knockdown assay to determine the top candidate siRNAs for targeting SARS-CoV-2; as described by our group in (12, 13). The top-candidate siRNAs (siUTR3 targeted to SL1 found in the parental full length and sub-genomic spliced transcripts (14), siUTR1 targeted to the 5′UTR/ORf1a, and siCS1-siCS4, targeted to ultra-conserved sites in RdRP of SARS-CoV1, CoV-2 and MERS and control siRNA, NC5 (FIG. 3, Tables F and G) (control siRNA NC5=SEQ ID NOS:11-12), will be selected and screened relative to the control and mock treated SARS-CoV-2 virus spike pseudotyped (21) and live virus infected VERO and human ACE2 over-expressing A549 lung epithelial cells (22). The effects of the siRNA suppression of SARS-CoV-2 expression will be determined by digital droplet qRT-PCR and luciferase expression as was done in FIG. 2. The top candidate suppressive siRNAs will be selected and chemically modified (FIG. 3) to stabilize and protect the siRNAs from degradation by serum and limit any potential for these siRNAs to induce any pro-inflammatory cytokines and innate immune responses. The chemically modified siRNAs will be generated and screened alone or in various combinations initially on SARS-CoV-2 virus spike pseudotyped (21) luciferase expressing and live virus infected VERO and human ACE2 over-expressing A549 lung epithelial cells (22) and the effects of the siRNAs suppression of reporter luciferase (FIG. 1B) contrasted with SARS-CoV-2 viral RNA expression as determined by luciferase expression and qRT-PCR analysis. These studies will determine the top candidate siRNAs with the optimal chemical modifications required as well as those siRNA combinations that potently target and repress SARS-CoV-2 expression. The top-candidate chemically-modified single and pool of siRNAs (multi-siRNAs) against SARS-CoV-2 will be selected and generated in stealth LNP formulations that will be assessed both in vitro using air liquid interface (ALI) vector infected A549 cell cultures (AIM 2) and in vivo (AIM 3).

A.1.3. Characterization of siRNA immune stimulatory effects. Some siRNAs have been found to be immune stimulatory, while we are embedding several chemical modifications into the top-candidate siRNAs (FIGS. 2 and 3), it is still possible that for some reason the candidates may activate the immune response. It has been previously shown that engagement of TLR7 by siRNAs results in the production of IFNα from pDCs (23, 24), and that TLR7 sensing of RNA is conserved between human and mouse (25). We will test the top-candidate chemically-modified and control unmodified siRNAs for the induction of IFNα and TNFα in murine and human PBMCs, mouse bone marrow macrophages, and macrophage/monocyte cell lines (RAW and U937). We have also developed an IFNβ digital droplet qRT-PCR assay for rapid readout of TLR induction and a qRT-PCR activation for interferon activation (26). We will use RAW and U937 cell systems as we know they are particularly sensitive to TLR activation, are excellent reporter cells, and will inform us that both human and mouse cells will react similarly. Collectively, these studies will determine which, if any, of the top-candidate siRNAs or pools of multiple targeted siRNAs to SARS-CoV-2 can instill an off-target immune response. Knowledge of any off-target immune responses can prove highly informative with regards to the final siRNA formulations packaged into the stealth LNPs.

A.1.4. The effects of siRNA on cellular transcriptomic programs. One concern with RNAi therapeutics is off-target effects (24). Chemical modifications can affect strand loading and also functionally reduce off-target effects (27, 28). We have designed all candidate siRNAs a priori using an in house bioinformatic tool and empirically selected siRNAs based on conservations between the various coronaviruses (e.g., Tables 1 and 2) and to insure no sequence complementarity to the human genome. We will also embed and screen various chemical modifications that reduce off-target effects and have been shown previously to be tolerated with LNP formulations (FIG. 3). However, despite our best efforts to minimize off-target issues, the inherent notion that all nucleic acid moieties when added to cells have some sort of off-target or secondary effect, still remains. To learn what genes may be affected by our candidate SARS-CoV-2 targeted chemically modified siRNAs, either alone or in combination, and the negative control will be transfected with the candidate chemically modified siRNAs (FIG. 5, Tables F and G) into both lung and liver cells, as both cell types may receive the stealth LNP siRNA formulations in vivo (FIGS. 4A-4C) and changes in the transcriptomic program of the cell determined at 30 min, 1, 4, 8, and 24 hours post-treatment for changes in transcriptome expression by RNA high-throughput deep sequencing of the isolated exosomes and RNA from the treated cultures. The resulting cellular RNAs will be mapped to the genome and differential expression in the various treated and control cultures determined using the Tophat-Cufflinks pipelines; described by our group in (29, 30). Collectively, these studies will delineate those genes and gene pathways affected by the top-candidate chemically modified SARS-CoV-2 siRNAs. Such an assessment will allow us to know those genes and gene networks that are affected by the various siRNAs and inform as to what off-target effects we may observe in vivo.

A.1.5. Significance of Aim 1: Aim 1 will identify the top-candidate siRNAs and cocktail of siRNAs that can functionally target inhibit SARS-CoV-2 and will identify what off-target effects, if any, are instilled in lung and liver treated cells treated with these siRNAs. These top-candidate siRNAs will be used in stealth LNP formulations (AIM 2) and assessed in vivo (AIM 3).

AIM 2: Develop and validate single and multiple siRNA packaged lipid nanoparticles formulated for intravenous and intranasal delivery. Optimized lipid nanoparticles containing chemically modified single and multiple siRNAs targeted to highly conserved sites in SARS-CoV-2 will be generated and assessed for targeted inhibition of virus expression in vitro and off-target effects from treating cells with these RNA therapeutics determined.

A.2.1. Rationale. This aim will determine the ability to generate therapeutic RNAi packaged stealth LNPs to deliver siRNA payloads and specifically repress SARS-CoV-2 expression. Determining the proper siRNA-LNP formulations capable of repressing SARS-CoV-2 expression will be important to determine the best candidate formulation for both stealth LNP IV and IN delivery in vivo treatment of the virus infection.

A.2.2. Develop and screen stealth siRNA lung directed LNPs for repression. The top candidate siRNAs and siRNA pools that impart minimal off-target and no unintended immune response will be selected from AIM 1 and packaged into stealth LNP formulations, described by our group in (6). These single and multiple siRNA-LNPs will be assessed for delivery of the candidate siRNAs and suppression of SARS-CoV-2 in infected cells in vitro. The methods for developing stealth LNPs are publicly available in and these methods (6, 19), generated by the McMillan and Morris labs and will be utilized to generate the candidate siRNA-LNP formulations. The resultant formulated siRNA-LNPs will be analyzed using the Nanoparticle Tracking Analysis (NTA), Dynamic light scattering (DLS) and Transmission Electron Microscopy (TEM). Collectively these assays will allow for the qualification and quantification of the formulated siRNA-LNPs. Next, the ensuing siRNA-LNPs will be exposed, in varying concentrations (ranging from 0 siRNA-LNPs/cell to 3.0×10\hat{ }5 siRNA-LNPs/cell, based on previous studies (19)) to cultured A549 and VERO cells containing the psi-check 2.1 SARS-CoV-2 target reporter system (FIGS. 1B-1C), as a sensitive proxy to measure siRNA repression of those sites targeted with the top-candidate siRNAs. Luciferase expression will be determined by both a luciferase assay; as described by our group in (12), and qRT-PCR from RNA of those cells treated with the siRNA-LNP formulations. These studies will determine the ability of siRNA-LNPs to deliver SARS-CoV-2 repressive siRNAs to model cells. While in vitro efficacy of LNPs does not always represent in vivo efficacy, this assay will inform as to the functionality of the various siRNA-LNP formulations. Those formulations that appear functional in these assays will be screened in SARS-CoV-2 infected A549 and VERO cells.

A.2.3. Stealth siRNA lung directed LNP repression of SARS-CoV-2 virus infection. The top-candidate SARS-CoV-2 targeted siRNA-LNP formulations, will be screened for their ability to repress virus expression in the context of virus infected ACE2 over-expressing A549 and VERO cells. The stealth SARS-CoV-2 directed siRNA-LNPs and untargeted siRNA-LNP and empty LNP controls will be exposed, in varying concentrations (determined in A.2.2.) to cultured SARS-CoV-2 infected A549 lung and VERO cells in vitro. The various treated cells will be assessed for virus RNA expression determined by qRT-PCR and viral protein expression determined by western blot of the differentially treated cells. We will also validate the respective siRNA-LNPs for repression of SARS-CoV-2 using by measuring cytopathic effect (32) on the cells (32) as a proxy for infection and plaque forming units PFU will also be used to determine infectious units following the various treatments, described in (33). Notably, the CPE and PFU assays offer a nice functional assay that is indicative of overall viral infectivity following the various treatments and do not depend on variations in antibody efficacy. Collectively, these studies should allow for the specificity, efficacy, and dosage of siRNA-LNPs to be determined relative to controls and focus in the final products siRNA-LNP and multiple-siRNA-LNPs to be assessed for targeted inhibition of SARS-CoV-2 virus expression in virus infected cells.

A.2.4. The effects of siRNA-LNP on cellular transcriptomic programs. Binding and internalization of the stealth anti-SARS-CoV-2 siRNA-LNP complexes in virus infected and control cells as well as the expression of anti-SARS-CoV-2 siRNAs in human cells may have differing and/or confounding effects on cellular transcriptional networks. For instance it is known that engaging the SARS-CoV-2 targeted ACE2 receptor can induce internal cellular changes, including in the transcriptomic program of the cell (34). While in A.1.4. above we will determine any off-target affected genes and gene networks with cells transfected with siRNAs, we will not know to what extent a variable off-target effect is observed when cells are treated with the final derived siRNA-LNP formulations.

To determine those transcriptional changes in target cells the various siRNA-LNP formulations, virus infected and uninfected cells (VERO and A549) and control Hep3B and Hep3G liver cells will be treated with the siRNA-LNP formulations and characterized at 30 minutes, 1, 4, 8, and 24 hours post-treatment for changes in transcriptome expression by Illumina RNA high-Throughput deep sequencing of the RNA from the differentially treated and control cultures. The resulting cellular RNAs will be mapped to the genome and differential expression in the various treated and control cultures determined using the Tophat-Cufflinks pipelines; described by our group in (29, 30). Collectively, these studies should delineate to what extent the top-candidate siRNA-LNP formulations affect various transcriptional networks in virus infected and uninfected cells. Particular attention will be paid to those gene networks perturbed between the virus infected and control uninfected cells.

A.2.5. Significance of Aim 2: Aim 2 will determine that siRNA-LNP formulations can functionally inhibit SARS-CoV-2 expression in infected human lung cells and determine the top-candidate formulations required to move forward to in vivo studies.

AIM 3: Assess anti-SARS-CoV-2 siRNA-LNPs in vivo. Contrast intravenous and intranasal administered anti-SARS-CoV-2 siRNA containing LNPs for targeted repression of SARS-CoV-2 luciferase containing and live virus infected K18-hACE2 mice. The specificity, potency, relative dosage and toxicology of siRNA-LNPs will be identified.

A.3.1. Rationale. This aim will identify the ability of the siRNA packaged stealth LNPs to deliver siRNA payloads and to virus infected cells in the lung in vivo and repress SARS-CoV-2 expression. This aim will confirm the functionality of the approach outlined in this proposal and determine which route of administration is best for functional repression of SARS-CoV-2 in vivo. The data generated in this aim will provide IND enabling data and the development of this approach as a bona fide therapeutic to treat SARS-CoV-2 infection and COVID-19 disease in humans.

A.3.1. In vivo characterization of stealth SARS-CoV-2 siRNA-LNP in luciferase lentiviral vector infected K18-hACE2 mice. To determine the dosage and functionality of the siRNA-LNPs developed here we will screen the various formulation in luciferase reporter virus infected K18-hACE2 mice (FIG. 1C). This assay will allow for rapid determination of the optimal concentrations of the siRNA-LNP formulations for targeted repression of their respective target gene. This is an important and needed experiment that will inform about the biodistribution, dosage, toxicology and pathology of the candidate siRNA-LNPs.

To determine the optimal dosage of siRNA-LNP in vivo we will first develop a lentiviral vector that is pseudotyped with the SARS-CoV-2 spike as well as a luciferase psi-check transgene system described by our group in (13), which contains both the SARS-CoV-2 siRNA target sites (e.g. 5′ UTR/SL1, 5′UTR/Orfa1 and RdRP, Tables F and G). This single round infectious vector (FIG. 1C) will be contrasted with control vectors, which contain the SARS-CoV-2 siRNA target sites (Tables F and G) in reverse orientation fused to GFP/luciferase. These spike pseudotyped vectors will be validated first in human ACE2 over-expressing A549 lung epithelial cells (22) and the ability to target these cells and repress luciferase by each SARS-CoV-2 directed siRNA-LNPs determined by treatment with the stealth SARS-CoV-2 targeted siRNA-LNPs. Once this system is confirmed in vitro (A.2.3), we will carry out in vivo studies. Spike-pseudotyped luciferase reporter lentiviral vectors will be used to infect K18-hACE2 mice and imaged for luciferin expression (COH fee for service Animal Resource Center). Once expression is determined the vector infected K18-hACE2 mice (10-12 weeks old male and female) will be treated with stealth siRNA-LNP by IV or IN injection, described in (6). The stealth siRNA-LNPs will be injected intravenously via the tail vein with 300 μl of D-PEGylated liposomes containing a range of 10-100 μg of siRNA; described in detail in (6), or administered IN with 200 μl of D-PEGylated liposomes containing a range of 10-100 μg of siRNA. The treated animals will be monitored at 24-96 hours after administration for luciferin expression from live animal imaging. After 96 hours the animals will be euthanized and the expression of SARS-CoV-2 luciferase fusion transcript expression determined in the lung by both luciferase imaging and qRT-PCR. The liver, spleen, and brain will also be collected and assessed by immunocytochemistry for the candidate anti-SARS-CoV-2 siRNAs, as an indication of tissues other than lung which are affected by the siRNA-LNP in vivo. The optimal concentration of both IV and IN administered siRNA-LNPs will be determined and assessed in virus infected K18-hACE2 mice in a biosafety lab 3 setting.

A.3.2. In vivo characterization of nebulized and stealth SARS-CoV-2 siRNA-LNP in virus infected K18-hACE2 mice.

To determine the potency and persistence of siRNA-LNP repression of SARS-CoV-2, SARS-CoV-2 virus intranasally inoculated K18-hACE2 mice will be treated with IV and IN siRNA-LNP and control formulations (determined in AIM 2). The animals will be treated with the predetermined concentration of SARS-CoV-2 directed single or multiple siRNA containing or control siRNA-LNP formulations (A.3.1.) either only on day 1 or on day 1 and day 4. The mice will be assessed on day 7 following the initial viral intranasal inoculation, which is day 6 following the initiation of treatment. The animals will be monitored for viral pathogenesis, as evident from weight loss, observations of lethargy, and labored breathing, and survivorship throughout the 7 day experiment. Past experiments have determined that control virus infected mice appear to become moribund by days 4-7 post-inoculation; as described in (35). On day 7 post-treatment the animals will be euthanized and whole-lung lavage carried out and virus measured in the lung by qRT-PCR and western blot, as described in (35). The lung, liver, spleen, brain and bone marrow of the euthanized animals will also be collected, and aliquots assessed for presence of the SARS-CoV-2 siRNAs or control siRNAs as well as the transcriptomic profiles of these tissues assessed by high-throughput exome RNA sequencing (described in A.2.4.) to determine any other tissues targeted and affected by the stealth LNP-siRNA treatment. Aliquots of blood will also be collected on day 7 prior to euthanizing the mice and a screen of immune-stimulatory cytokines carried out by ELISA; as described in and immunocytochemistry carried out for the candidate anti-SARS-CoV-2 siRNAs, as an indication of tissues other than lung which are affected by the siRNA-LNP in vivo.

Collectively, the results of this experiment will provide the efficacy, safety and feasibility of the approach outlined in this disclosure and delineate the relative effects of IV relative to IN delivery of stealth siRNA-LNP formulations as a therapeutic for treating COVID-19.

A.3.3. Significance of AIM 3: AIM3 marks an important and significant step in our strategy to deliver anti-SARS-CoV-2 therapeutic siRNA-LNPs to the lung of infected individuals. These in vivo studies will establish the proof-of-concept for our strategy to deliver anti-SARS-CoV-2 siRNA-LNPs to the lungs and serve of key IND enabling data required to approach the FDA to develop this therapeutic as a means to repress virus expression and treat COVID-19.

Outcomes and Significance: The impact of this work is that it will result in the development and commercialization of a lung targeted siRNA-LNP approach to repress the SARS-CoV-2 infection. Our project is a highly novel and impactful approach that is modular, meaning that this approach can be applied for all coronavirus infections as well as other viruses that afflict humans by replication in the lungs, e.g. MERS and influenza. Notably, we are using siRNAs which have been designed a priori to target SARS and MERS, coronaviruses with significant potential to cause pandemics. The significance of this work is therefore that it will result in the development of a unique siRNA-LNP strategy that will be a safe and effective therapy for SARS-CoV-2 and other coronaviruses.

### Example 5

RNA interference (26) is a mechanism of action imbued in mammalian cells that can specifically turn off the production of proteins in cells in a sequence-specific and potent manner. In general, RNAi works via the introduction of small-interfering RNAs (siRNA, 15-30 bp of double stranded RNA) that specifically target mRNAs via sequence complementarity, causing their subsequent degradation (27). RNAi has been found in previous studies to potently repress SARS-CoV-1 replication and infection (28). Both RNAi and antisense non-coding RNA modes of gene repression will be useful in repressing SARS-CoV-2 and amenable to exosome mediated delivery approaches.

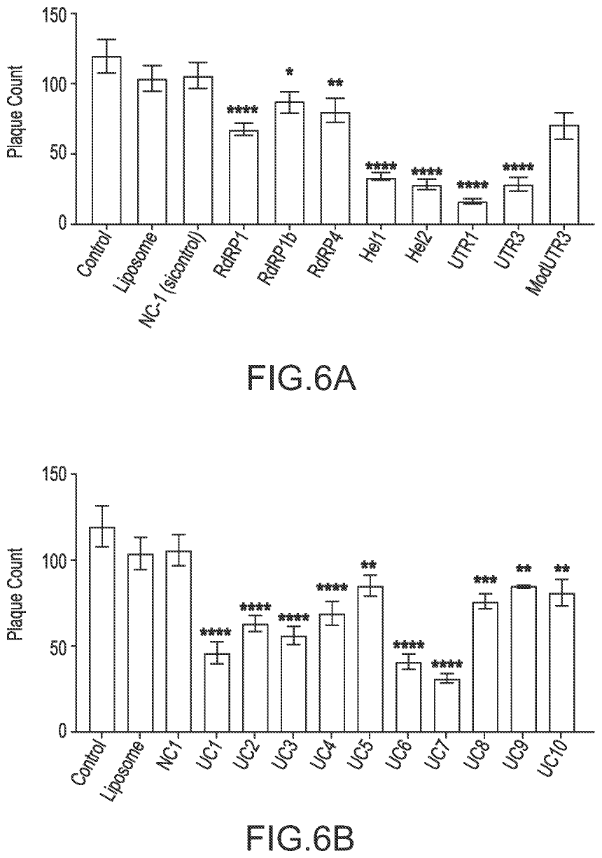

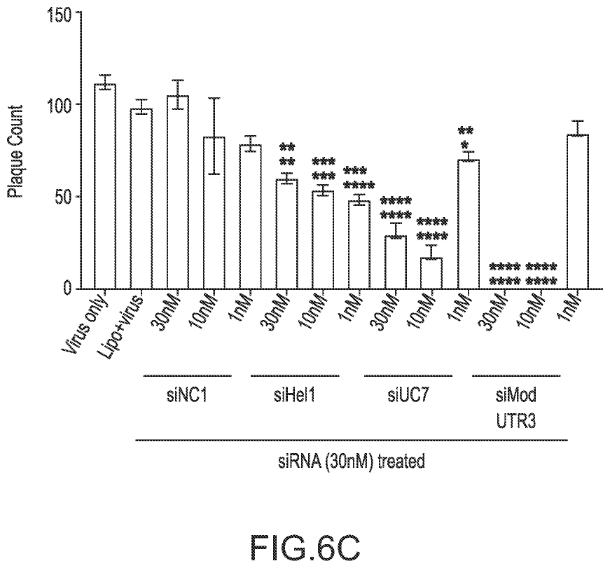

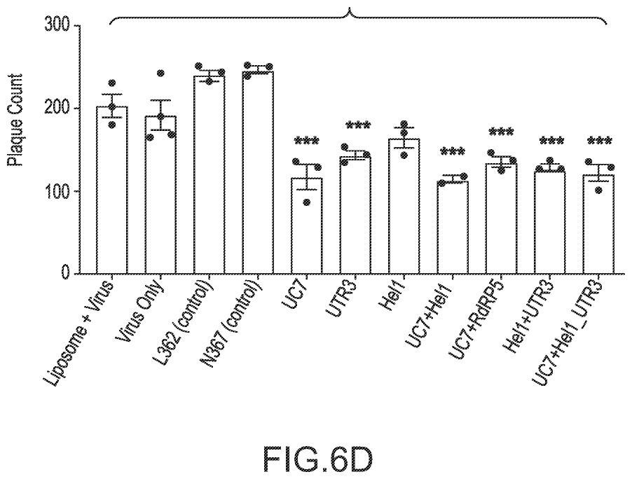

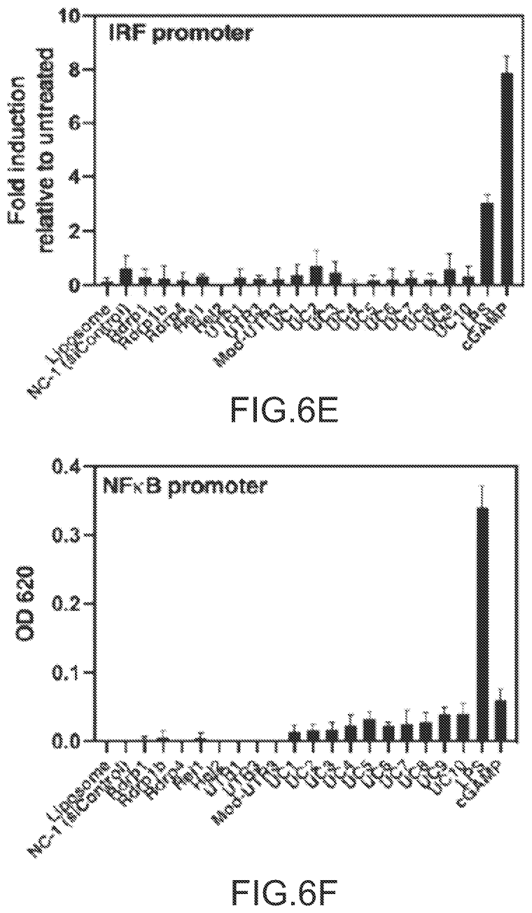

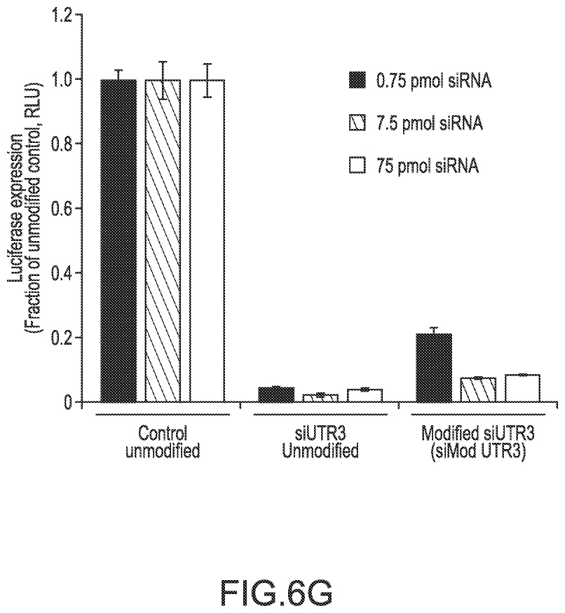

The advantage to RNAi in targeting viruses is that multiple siRNAs can be developed targeted to conserved loci or key viral genes to avoid the emergence of viral resistance. SiRNAs targeted to the RNA dependent RNA Polymerase (RdRp), helicase, and 5′ untranslated leader region (5′UTR) were selected and screened along with bioinformatically selected ultra-conserved regions. The ultra-conserved siRNAs were discovered from characterizing the 29,903 bp RNA genome of SARS-CoV-2 for structural features and sequence conservation relative to SARS-COV-1 and MERS. The constraints were required to lack secondary structure, contain sequence conservation with SARS-CoV-1, bat coronaviruses, and vertebrate coronaviruses. The absence of seed sequences in the human transcriptome was also a requirement. From this bioinformatic approach, the siRNAs shown in Tables D and E were designed and assessed. Each siRNA screened had some effect on SARS-COV-2 in vitro (FIGS. 6A-6B) with siRNA Hel2, siUC7 and siUTR3 demonstrating the most potent and dose dependent repression of virus expression (FIG. 6C).

Tables D and E identify siRNA's targeted to RdRP, Helicase, the 5′UTR, and ultra-conserved regions found from characterizing the 29,903 bp RNA genome of SARS-CoV-2 for structural features and sequence conservation. Roughly 9,500 candidate siRNAs were prioritized and generated by the OligoWalk & DSIR pipelines (34, 35) in addition to 163 experimentally validated SARS-CoV-1 siRNAs already published. We restricted candidates to those that fall in windows defined by the lack of secondary structure and contain sequence conservation with SARS-CoV-1, in bat coronaviruses and in vertebrate coronaviruses and assessed for the absence of seed sequences in the human transcriptome. The top 10 candidates are siUC1-siUC10 (siRNA sequences shown in Table E).

SARS-COV-2 is able to rapidly emerge mutations that make the virus refractory to antibody targeting (1), and it is well known in the case of HIV, an RNA virus, that single siRNA targeting results in the emergence of viral resistance (2) while combinations of siRNAs have been shown to be refractory to the emergence of viral resistant variants (3). To ascertain if combining siRNAs to target multiple mRNA at once, while maintaining the overall siRNA concentration, we selected the top 3 siRNAs (i.e., siUTR3, siUC7, and siHel2) alone and in various combinations. Interestingly, we find here that combinations of siRNAs offered the same viral knockdown as was observed with single targeted siRNAs, even though the concentration of individual siRNAs in each commination were lower the single doses (50% for 2 siRNAs and 33% for 3 siRNAs)(FIG. 6D). These data indicate that pooled combinations of siRNAs can be developed to repress SARS-COV-2 as a means to maintain viral repression in the presence of a rapidly mutating virus.

An important feature of the selected siRNAs was the requirement that they are not immunostimulatory as this is clearly a feature of COVID-19 disease that is problematic at advanced stages. The immunostimulatory aspects of these siRNAs was tested using the THP1 DUAL™ cell system, a well-recognized standard to measure immunostimulation. This assay measures both IRF and NFkB promoter stimulation simultaneously as an indicator of immunostimulation. The THP-1 cells were transfected with siRNA (30 nM) and stimulation assayed at 24 hours. No significant immunostimulation was observed with any siRNA while the positive controls (LPS and cGAMP) were highly stimulatory (FIGS. 6E-6F). Collectively, these data indicate that the candidate siRNAs are safe with regards to immunostimulation.

Small interfering RNAs can be stabilized with chemical modifications, which results in a long-term expression and more potent repression (4). We selected siUTR3, as this target site is stem loop 1, a highly conserved region in the 5′UTR that is required for downstream transcriptional processing and expression of the many viral RNAs (5). We generated 2′ O-methyl modifications chemical modifications into siUTR3 (FIG. 3A) and found that siUTR3 tolerated these stabilizing chemical modifications (FIG. 6G), indicating that this siRNA is an excellent candidate as a therapeutic for targeting the repression of SARS-COV-2.

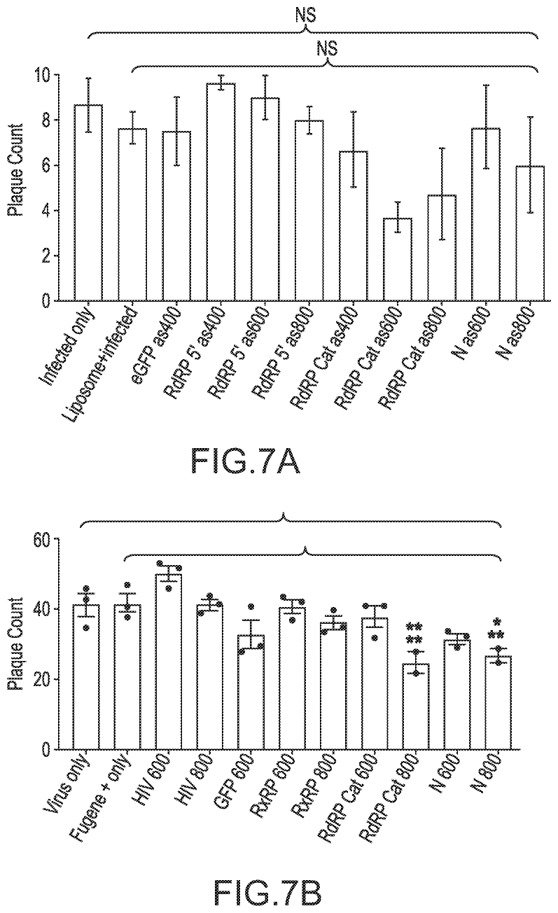

As an alternative strategy to RNAi, we sought to interrogate to what extent long antisense non-coding RNAs may repress SARS-CoV-2. We screened antisense RNAs ranging from 400-800 bp in length targeted to the RdRP region of SARS-COV-2. Both as600 and as 800 bp RNAs repressed SARS-COV-2 virus infection (FIG. 7A) with the 800 bp antisense RNA directed to RdRP found to reproducibly repress SARS-CoV-2 (FIG. 7B). The mechanism of action for how these antisense RNAs repress virus expression remains unclear at this time but, without intending to be bound by any theory of the mechanism of action, may involve ADAR directed mutations into RdRP. Collectively, these data support earlier findings that SARS-CoV-2, like other coronaviruses (32, 33), is also susceptible to RNAi and that the 5′ UTR and RdRP serve as a distinct region that is highly susceptible to RNA mediated modes of repression.

### References

1. Ji Y, Ma Z, Peppelenbosch M P, Pan Q. Lancet Glob Health. 2020. Epub 2020/02/29. doi: 10.1016/S2214-109X(20)30068-1. PubMed PMID: 32109372. 2. Sun M L, Yang J M, Sun Y P, Su G H. Zhonghua Jie He He Hu Xi Za Zhi. 2020; 43(0):E014. Epub 2020/02/16. doi: 10.3760/cma.j.issn.1001-0939.2020.0014. PubMed PMID: 32061198. 3. Fehr A R, Perlman S. Coronaviruses: an overview of their replication and pathogenesis. Methods Mol Biol. 2015; 1282:1-23. Epub 2015/02/28. doi: 10.1007/978-1-4939-2438-7_1. PubMed PMID: 25720466; PMCID: PMC4369385. 4. Casucci M, Falcone L, Camisa B, Norelli M, Porcellini S, Stornaiuolo A, Ciceri F, Traversari C, Bordignon C, Bonini C, Bondanza A. Extracellular NGFR Spacers Allow Efficient Tracking and Enrichment of Fully Functional CAR-T Cells Co-Expressing a Suicide Gene. Front Immunol. 2018; 9:507. Epub 2018/04/06. doi: 10.3389/fimmu.2018.00507. PubMed PMID: 29619024; PMCID: PMC5871667. 5. Sohrab S S, El-Kafrawy S A, Mirza Z, Kamal M A, Azhar E L Design and Delivery of Therapeutic siRNAs: Application to MERS-Coronavirus. Curr Pharm Des. 2018; 24(1):62-77. Epub 2017/11/10. doi: 10.2174/1381612823666171109112307. PubMed PMID: 29119921. 6. McCaskill J, Singhania R, Burgess M, Allavena R, Wu S, Blumenthal A, McMillan N A. Efficient Biodistribution and Gene Silencing in the Lung epithelium via Intravenous Liposomal Delivery of siRNA. Mol Ther Nucleic Acids. 2013; 2:e96. Epub 2013/06/06. doi: 10.1038/mtna.2013.22. PubMed PMID: 23736774; PMCID: PMC3696903. 7. Khairuddin N, Blake S J, Firdaus F, Steptoe R J, Behlke M A, Hertzog P J, McMillan N A. In vivo comparison of local versus systemic delivery of immunostimulating siRNA in HPV-driven tumours. Immunol Cell Biol. 2014; 92(2):156-63. Epub 2013/11/13. doi: 10.1038/icb.2013.75. PubMed PMID: 24217808. 8. Wu C J, Huang H W, Liu C Y, Hong C F, Chan Y L. Inhibition of SARS-CoV replication by siRNA. Antiviral Res. 2005; 65(1):45-8. Epub 2005/01/18. doi: 10.1016/j.antiviral.2004.09.005. PubMed PMID: 15652970. 9. Shi Y, Yang D H, Xiong J, Jia J, Huang B, Jin Y X. Inhibition of genes expression of SARS coronavirus by synthetic small interfering RNAs. Cell Res. 2005; 15(3):193-200. Epub 2005/03/23. doi: 10.1038/sj.cr.7290286. PubMed PMID: 15780182. 10. Li B J, Tang Q, Cheng D, Qin C, Xie F Y, Wei Q, Xu J, Liu Y, Zheng B J, Woodle M C, Zhong N, Lu P Y. Using siRNA in prophylactic and therapeutic regimens against SARS coronavirus in Rhesus macaque. Nat Med. 2005; 11(9):944-51. Epub 2005/08/24. doi: 10.1038/nm1280. PubMed PMID: 16116432; PMCID: PMC7095788. 11. Chi X, Gatti P, Papoian T. Safety of antisense oligonucleotide and siRNA-based therapeutics. Drug Discov Today. 2017; 22(5):823-33. Epub 2017/02/06. doi: 10.1016/j.drudis.2017.01.013. PubMed PMID: 28159625. 12. Barichievy S, Saayman S, von Eije K J, Morris K V, Arbuthnot P, Weinberg M S. The inhibitory efficacy of RNA POL III-expressed long hairpin RNAs targeted to untranslated regions of the HIV-1 5′ long terminal repeat. Oligonucleotides. 2007; 17(4):419-31. Epub 2007/09/28. doi: 10.1089/oli.2007.0095. PubMed PMID: 17896874. 13. Weinberg M S, Barichievy S, Schaffer L, Han J, Morris K V. An RNA targeted to the HIV-1 LTR promoter modulates indiscriminate off-target gene activation. Nucleic acids research. 2007; 35(21):7303-12. PubMed PMID: 17959645. 14. Sola I, Almazan F, Zuniga S, Enjuanes L. Continuous and Discontinuous RNA Synthesis in Coronaviruses. Annu Rev Virol. 2015; 2(1):265-88. Epub 2016/03/10. doi: 10.1146/annurev-virology-100114-055218. PubMed PMID: 26958916; PMCID: PMC6025776. 15. DeVincenzo J, Lambkin-Williams R, Wilkinson T, Cehelsky J, Nochur S, Walsh E, Meyers R, Gollob J, Vaishnaw A. A randomized, double-blind, placebo-controlled study of an RNAi-based therapy directed against respiratory syncytial virus. Proceedings of the National Academy of Sciences of the United States of America. 2010; 107(19):8800-5. Epub 2010/04/28. doi: 10.1073/pnas.0912186107. PubMed PMID: 20421463; PMCID: PMC2889365. 16. Shim M S, Kwon Y J. Efficient and targeted delivery of siRNA in vivo. FEBS J. 2010; 277(23):4814-27. Epub 2010/11/17. doi: 10.1111/j.1742-4658.2010.07904.x. PubMed PMID: 21078116. 17. Youngren-Ortiz S R, Gandhi N S, Espana-Serrano L, Chougule M B. Aerosol Delivery of siRNA to the Lungs. Part 2: Nanocarrier-based Delivery Systems. Kona. 2017; 34:44-69. Epub 2017/04/11. doi: 10.14356/kona.2017005. PubMed PMID: 28392618; PMCID: PMC5381822. 18. Gu J, Korteweg C. Pathology and pathogenesis of severe acute respiratory syndrome. Am J Pathol. 2007; 170(4):1136-47. Epub 2007/03/30. doi: 10.2353/ajpath.2007.061088. PubMed PMID: 17392154; PMCID: PMC1829448. 19. Villamizar O, Waters S A, Scott T, Saayman S, Grepo N, Urak R, Davis A, Jaffe A, Morris K V. Targeted Activation of Cystic Fibrosis Transmembrane Conductance Regulator. Molecular therapy: the journal of the American Society of Gene Therapy. 2019; 27(10):1737-48. Epub 2019/08/07. doi: 10.1016/j.ymthe.2019.07.002. PubMed PMID: 31383454; PMCID: PMC6822231. 20. Olajuyin A M, Olajuyin A K, Wang Z, Zhao X, Zhang X. CD146 T cells in lung cancer: its function, detection, and clinical implications as a biomarker and therapeutic target. Cancer Cell Int. 2019; 19:247. Epub 2019/10/02. doi: 10.1186/s12935-019-0969-9. PubMed PMID: 31572064; PMCID: PMC6761715. 21. Menachery V D, Yount B L, Jr., Debbink K, Agnihothram S, Gralinski L E, Plante J A, Graham R L, Scobey T, Ge X Y, Donaldson E F, Randell S H, Lanzavecchia A, Marasco W A, Shi Z L, Baric R S. A SARS-like cluster of circulating bat coronaviruses shows potential for human emergence. Nat Med. 2015; 21(12):1508-13. Epub 2015/11/10. doi: 10.1038/nm.3985. PubMed PMID: 26552008; PMCID: PMC4797993. 22. Qian Y R, Guo Y, Wan H Y, Fan L, Feng Y, Ni L, Xiang Y, Li Q Y. Angiotensin-converting enzyme 2 attenuates the metastasis of non-small cell lung cancer through inhibition of epithelial-mesenchymal transition. Oncol Rep. 2013; 29(6):2408-14. Epub 2013/04/03. doi: 10.3892/or.2013.2370. PubMed PMID: 23545945. 23. Hornung V, Guenthner-Biller M, Bourquin C, Ablasser A, Schlee M, Uematsu S, Noronha A, Manoharan M, Akira S, de Fougerolles A, Endres S, Hartmann G. Sequence-specific potent induction of IFN-alpha by short interfering RNA in plasmacytoid dendritic cells through TLR7. Nat Med. 2005; 11(3):263-70. Epub 2005/02/22. doi: 10.1038/nm1191. PubMed PMID: 15723075. 24. Judge A D, Sood V, Shaw J R, Fang D, McClintock K, MacLachlan I. Sequence-dependent stimulation of the mammalian innate immune response by synthetic siRNA. Nat Biotechnol. 2005; 23(4):457-62. Epub 2005/03/22. doi: 10.1038/nbt1081. PubMed PMID: 15778705. 25. Gantier M P, Williams B R. The response of mammalian cells to double-stranded RNA. Cytokine Growth Factor Rev. 2007; 18(5-6):363-71. Epub 2007/08/19. doi: 10.1016/j.cytogfr.2007.06.016. PubMed PMID: 17698400; PMCID: PMC2084215. 26. Pollard K M, Cauvi D M, Toomey C B, Morris K V, Kono D H. Interferon-gamma and systemic autoimmunity. Discov Med. 2013; 16(87):123-31. Epub 2013/09/04. PubMed PMID: 23998448; PMCID: PMC3934799. 27. Varley A J, Hammill M L, Salim L, Desaulniers J P. Effects of Chemical Modifications on siRNA Strand Selection in Mammalian Cells. Nucleic Acid Ther. 2020. Epub 2020/03/17. doi: 10.1089/nat.2020.0848. PubMed PMID: 32175808. 28. Judge A D, Bola G, Lee A C, MacLachlan I. Design of noninflammatory synthetic siRNA mediating potent gene silencing in vivo. Molecular therapy: the journal of the American Society of Gene Therapy. 2006; 13(3):494-505. Epub 2005/12/14. doi: 10.1016/j.ymthe.2005.11.002. PubMed PMID: 16343994. 29. Hewson C, Capraro D, Burdach J, Whitaker N, Morris K V. Extracellular vesicle associated long non-coding RNAs functionally enhance cell viability. Noncoding RNA Res. 2016; 1(1):3-11. Epub 2017/01/17. doi: 10.1016/j.ncrna.2016.06.001. PubMed PMID: 28090596; PMCID: PMC5228635. 30. Trakman L, Hewson C, Burdach J, Morris K V. RNA Directed Modulation of Phenotypic Plasticity in Human Cells. PloS one. 2016; 11(4):e0152424. Epub 2016/04/16. doi: 10.1371/journal.pone.0152424. PubMed PMID: 27082860; PMCID: PMC4833343. 31. Ackley A, Lenox A, Stapleton K, Knowling S, Lu T, Sabir K S, Vogt P K, Morris K V. An Algorithm for Generating Small RNAs Capable of Epigenetically Modulating Transcriptional Gene Silencing and Activation in Human Cells. Mol Ther Nucleic Acids. 2013; 2:e104. Epub 2013/07/11. doi: 10.1038/mtna.2013.33. PubMed PMID: 23839098; PMCID: PMC3731886. 32. Sempere L F, Cole C N, McPeek M A, Peterson K J. The phylogenetic distribution of metazoan microRNAs: insights into evolutionary complexity and constraint. J Exp Zool B Mol Dev Evol. 2006; 306(6):575-88. Epub 2006/07/14. doi: 10.1002/jez.b.21118. PubMed PMID: 16838302. 33. Sims A C, Baric R S, Yount B, Burkett S E, Collins P L, Pickles R J. Severe acute respiratory syndrome coronavirus infection of human ciliated airway epithelia: role of ciliated cells in viral spread in the conducting airways of the lungs. J Virol. 2005; 79(24):15511-24. Epub 2005/11/25. doi: 10.1128/JVI.79.24.15511-15524.2005. PubMed PMID: 16306622; PMCID: PMC1316022. 34. Kamel A S, Abdelkader N F, Abd El-Rahman S S, Emara M, Zaki H F, Khattab M M. Stimulation of ACE2/ANG(1-7)/Mas Axis by Diminazene Ameliorates Alzheimer's Disease in the D-Galactose-Ovariectomized Rat Model: Role of PI3K/Akt Pathway. Mol Neurobiol. 2018; 55(10):8188-202. Epub 2018/03/09. doi: 10.1007/s12035-018-0966-3. PubMed PMID: 29516284. 35. McCray P B, Jr., Pewe L, Wohlford-Lenane C, Hickey M, Manzel L, Shi L, Netland J, Jia H P, Halabi C, Sigmund C D, Meyerholz D K, Kirby P, Look D C, Perlman S. Lethal infection of K18-hACE2 mice infected with severe acute respiratory syndrome coronavirus. J Virol. 2007; 81(2):813-21. Epub 2006/11/03. doi: 10.1128/JVI.02012-06. PubMed PMID: 17079315; PMCID: PMC1797474. 36. Almazan F, Sola I, Zuniga S, Marquez-Jurado S, Morales L, Becares M, Enjuanes L. Coronavirus reverse genetic systems: infectious clones and replicons. Virus Res. 2014; 189:262-70. Epub 2014/06/17. doi: 10.1016/j.virusres.2014.05.026. PubMed PMID: 24930446; PMCID: PMC4727449.

### References for Example 5

1. Weisblum Y, Schmidt F, Zhang F, DaSilva J, Poston D, Lorenzi J C, Muecksch F, Rutkowska M, Hoffmann H H, Michailidis E, Gaebler C, Agudelo M, Cho A, Wang Z, Gazumyan A, Cipolla M, Luchsinger L, Hillyer C D, Caskey M, Robbiani D F, Rice C M, Nussenzweig M C, Hatziioannou T, Bieniasz P D. Escape from neutralizing antibodies by SARS-CoV-2 spike protein variants. Elife. 2020; 9. Epub 2020/10/29. doi: 10.7554/eLife.61312. PubMed PMID: 33112236; PMCID: PMC7723407. 2. Das A T, Brummelkamp T R, Westerhout E M, Vink M, Madiredjo M, Bernards R, Berkhout B. Human immunodeficiency virus type 1 escapes from RNA interference-mediated inhibition. J Virol. 2004; 78(5):2601-5. Epub 2004/02/14. doi: 10.1128/jvi.78.5.2601-2605.2004. PubMed PMID: 14963165; PMCID: PMC369246. 3. Liu Y P, Haasnoot J, ter Brake O, Berkhout B, Konstantinova P. Inhibition of HIV-1 by multiple siRNAs expressed from a single microRNA polycistron. Nucleic Acids Res. 2008; 36(9):2811-24. Epub 2008/03/19. doi: 10.1093/nar/gkn109. PubMed PMID: 18346971; PMCID: PMC2396423. 4. Selvam C, Mutisya D, Prakash S, Ranganna K, Thilagavathi R. Therapeutic potential of chemically modified siRNA: Recent trends. Chem Biol Drug Des. 2017; 90(5):665-78. Epub 2017/04/06. doi: 10.1111/cbdd.12993. PubMed PMID: 28378934; PMCID: PMC5935465. 5. Miao Z, Tidu A, Eriani G, Martin F. Secondary structure of the SARS-CoV-2 5′-UTR. RNA Biol. 2020:1-10. Epub 2020/09/24. doi: 10.1080/15476286.2020.1814556. PubMed PMID: 32965173; PMCID: PMC7544965.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A nucleic acid comprising:
(i) 15 nucleotides to 30 nucleotides and capable of hybridizing to SEQ ID NO:224, SEQ ID NO:231, SEQ ID NO:124, SEQ ID NO:223, SEQ ID NO:225, SEQ ID NO:230, or SEQ ID NO:123;
(ii) at least 10 nucleotides and capable of hybridizing to any one of SEQ ID NOS:13-81; any one of SEQ ID NOS:100-124; any one of SEQ ID NOS:223-235, or any one of SEQ ID NOS:258-265; or
(iii) SEQ ID NO:1 hybridized to SEQ ID NO:3; SEQ ID NO:2 hybridized to SEQ ID NO:4; SEQ ID NO:250 hybridized to SEQ ID NO:251; SEQ ID NO:220 hybridized to SEQ ID NO:221; SEQ ID NO:5 hybridized to SEQ ID NO:7; SEQ ID NO:6 hybridized to SEQ ID NO:8; SEQ ID NO:238 hybridized to SEQ ID NO:239; SEQ ID NO:248 hybridized to SEQ ID NO:249; SEQ ID NO:218 hybridized to SEQ ID NO:219; SEQ ID NO:1; SEQ ID NO:3; SEQ ID NO:2; SEQ ID NO:4; SEQ ID NO:5; SEQ ID NO:7; SEQ ID NO:6; SEQ ID NO:8; SEQ ID NO:250; SEQ ID NO:251; SEQ ID NO:220; SEQ ID NO:221; SEQ ID NO:238; SEQ ID NO:239; SEQ ID NO:248; SEQ ID NO:249; SEQ ID NO:218; SEQ ID NO:219; SEQ ID NO:82 hybridized to SEQ ID NO:83; SEQ ID NO:82; SEQ ID NO:83; SEQ ID NO:84 hybridized to SEQ ID NO:85; SEQ ID NO:84; SEQ ID NO:85; SEQ ID NO:86 hybridized to SEQ ID NO:87; SEQ ID NO:86; SEQ ID NO:87; SEQ ID NO:88 hybridized to SEQ ID NO:89; SEQ ID NO:88; SEQ ID NO:89; SEQ ID NO:90 hybridized to SEQ ID NO:91; SEQ ID NO:90; SEQ ID NO:91; SEQ ID NO:92 hybridized to SEQ ID NO:93; SEQ ID NO:92; SEQ ID NO:93; SEQ ID NO:94 hybridized to SEQ ID NO:95; SEQ ID NO:94; SEQ ID NO:95; SEQ ID NO:96 hybridized to SEQ ID NO:97; SEQ ID NO:96; SEQ ID NO:97; SEQ ID NO:214 hybridized to SEQ ID NO:215; SEQ ID NO:214; SEQ ID NO:215; SEQ ID NO:216 hybridized to SEQ ID NO:217; SEQ ID NO:216; SEQ ID NO:217; SEQ ID NO:236 hybridized to SEQ ID NO:237; SEQ ID NO:236; SEQ ID NO:237; SEQ ID NO:240 hybridized to SEQ ID NO:241; SEQ ID NO:240; SEQ ID NO:241; SEQ ID NO:242 hybridized to SEQ ID NO:243; SEQ ID NO:242; SEQ ID NO:243; SEQ ID NO:244 hybridized to SEQ ID NO:245; SEQ ID NO:244; SEQ ID NO:245; SEQ ID NO:246 hybridized to SEQ ID NO:247; SEQ ID NO:246; SEQ ID NO:247; SEQ ID NO:252 hybridized to SEQ ID NO:253; SEQ ID NO:252; SEQ ID NO:253; SEQ ID NO:254 hybridized to SEQ ID NO:255; SEQ ID NO:254; SEQ ID NO:255; SEQ ID NO:256 hybridized to SEQ ID NO:257; SEQ ID NO:256; SEQ ID NO:257; SEQ ID NO:125 hybridized to SEQ ID NO:126; SEQ ID NO:126 hybridized to SEQ ID NO:127; SEQ ID NO:104 hybridized to SEQ ID NO:128; SEQ ID NO:129 hybridized to SEQ ID NO:130; SEQ ID NO:131 hybridized to SEQ ID NO:132; SEQ ID NO:133 hybridized to SEQ ID NO:134; SEQ ID NO:135 hybridized to SEQ ID NO:136; SEQ ID NO:137 hybridized to SEQ ID NO:138; SEQ ID NO:139 hybridized to SEQ ID NO:140; SEQ ID NO:141 hybridized to SEQ ID NO:142; SEQ ID NO:143 hybridized to SEQ ID NO:144; SEQ ID NO:145 hybridized to SEQ ID NO:146; SEQ ID NO:147 hybridized to SEQ ID NO:148; SEQ ID NO:149 hybridized to SEQ ID NO:150; SEQ ID NO:115 hybridized to SEQ ID NO:151; SEQ ID NO:152 hybridized to SEQ ID NO:153; SEQ ID NO:154 hybridized to SEQ ID NO:155; SEQ ID NO:156 hybridized to SEQ ID NO:157; SEQ ID NO:158 hybridized to SEQ ID NO:159; SEQ ID NO:160 hybridized to SEQ ID NO:161; SEQ ID NO:162 hybridized to SEQ ID NO:163; SEQ ID NO:125; SEQ ID NO:126; SEQ ID NO:126; SEQ ID NO:127; SEQ ID NO:104; SEQ ID NO:128; SEQ ID NO:129; SEQ ID NO:130; SEQ ID NO:131; SEQ ID NO:132; SEQ ID NO:133; SEQ ID NO:134; SEQ ID NO:135; SEQ ID NO:136; SEQ ID NO:137; SEQ ID NO:138; SEQ ID NO:139; SEQ ID NO:140; SEQ ID NO:141; SEQ ID NO:142; SEQ ID NO:143; SEQ ID NO:144; SEQ ID NO:145; SEQ ID NO:146; SEQ ID NO:147; SEQ ID NO:148; SEQ ID NO:149; SEQ ID NO:150; SEQ ID NO:115; SEQ ID NO:151; SEQ ID NO:152; SEQ ID NO:153; SEQ ID NO:154; SEQ ID NO:155; SEQ ID NO:156; SEQ ID NO:157; SEQ ID NO:158; SEQ ID NO:159; SEQ ID NO:160; SEQ ID NO:161; SEQ ID NO:162; SEQ ID NO:163; SEQ ID NO:164 hybridized to SEQ ID NO:165; SEQ ID NO:166 hybridized to SEQ ID NO:167; SEQ ID NO:168 hybridized to SEQ ID NO:169; SEQ ID NO:170 hybridized to SEQ ID NO:171; SEQ ID NO:172 hybridized to SEQ ID NO:173; SEQ ID NO:174 hybridized to SEQ ID NO:175; SEQ ID NO:176 hybridized to SEQ ID NO:177; SEQ ID NO:178 hybridized to SEQ ID NO:179; SEQ ID NO:180 hybridized to SEQ ID NO:181; SEQ ID NO:182 hybridized to SEQ ID NO:183; SEQ ID NO:184 hybridized to SEQ ID NO:185; SEQ ID NO:186 hybridized to SEQ ID NO:187; SEQ ID NO:188 hybridized to SEQ ID NO:189; SEQ ID NO:190 hybridized to SEQ ID NO:191; SEQ ID NO:192 hybridized to SEQ ID NO:193; SEQ ID NO:194 hybridized to SEQ ID NO:195; SEQ ID NO:196 hybridized to SEQ ID NO:197; SEQ ID NO:198 hybridized to SEQ ID NO:199; SEQ ID NO:200 hybridized to SEQ ID NO:201; SEQ ID NO:202 hybridized to SEQ ID NO:203; SEQ ID NO:204 hybridized to SEQ ID NO:205; SEQ ID NO:164; SEQ ID NO:165; SEQ ID NO:166; SEQ ID NO:167; SEQ ID NO:168; SEQ ID NO:169; SEQ ID NO:170; SEQ ID NO:171; SEQ ID NO:172; SEQ ID NO:173; SEQ ID NO:174; SEQ ID NO:175; SEQ ID NO:176; SEQ ID NO:177; SEQ ID NO:178; SEQ ID NO:179; SEQ ID NO:180; SEQ ID NO:181; SEQ ID NO:182; SEQ ID NO:183; SEQ ID NO:184; SEQ ID NO:185; SEQ ID NO:186; SEQ ID NO:187; SEQ ID NO:188; SEQ ID NO:189; SEQ ID NO:190; SEQ ID NO:191; SEQ ID NO:192; SEQ ID NO:193; SEQ ID NO:194; SEQ ID NO:195; SEQ ID NO:196; SEQ ID NO:197; SEQ ID NO:198; SEQ ID NO:199; SEQ ID NO:200; SEQ ID NO:201; SEQ ID NO:202; SEQ ID NO:203; SEQ ID NO:204; SEQ ID NO:205; SEQ ID NO:206 hybridized to SEQ ID NO:207; SEQ ID NO:208 hybridized to SEQ ID NO:209; SEQ ID NO:210 hybridized to SEQ ID NO:211; SEQ ID NO:212 hybridized to SEQ ID NO:213; SEQ ID NO:206; SEQ ID NO:207; SEQ ID NO:208; SEQ ID NO:209; SEQ ID NO:210; SEQ ID NO:211; SEQ ID NO:212; SEQ ID NO:213; SEQ ID NO:266, SEQ ID NO:267, SEQ ID NO:268, SEQ ID NO:269, SEQ ID NO:270, SEQ ID NO:271, SEQ ID NO:272, or SEQ ID NO:273.

2. The nucleic acid of claim 1, wherein the nucleic acid of (i) or (ii) comprises 20 nucleotides to 25 nucleotides.

3-48. (canceled)

49. A nucleic acid having at least 80% sequence identify to SEQ ID NO:266, SEQ ID NO:267, SEQ ID NO:268, SEQ ID NO:269, SEQ ID NO:270, SEQ ID NO:271, SEQ ID NO:272, or SEQ ID NO:273.

50-51. (canceled)

52. The nucleic acid of claim 1, wherein the nucleic acid sequence comprises a modified base, a modified sugar, a modified phosphate, or a combination of two or more thereof.

53. The nucleic acid of claim 52, wherein the modified base is 2′O-Methyl modified base, a 2′O-methoxyethoxy modified base, a 2′fluoro modified base, a 5-methyl-modified cytidine, or pseudouridine.

54. (canceled)

55. The nucleic acid of claim 52, wherein the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite.

56. The nucleic acid of claim 55, wherein the modified phosphate is phosphorothioate.

57-58. (canceled)

59. The nucleic acid of claim 1, wherein the nucleic acid is antisense RNA.

60. The nucleic acid of claim 1, wherein the nucleic acid is siRNA.

61. A lipid nanoparticle comprising a lipid and the nucleic acid of claim 1.

62. The lipid nanoparticle of claim 61, wherein the lipid comprises a cationic lipid, a non-cationic lipid, a conjugated lipid that prevents aggregation of the nanoparticle, or a combination of two or more thereof.

63. A pharmaceutical composition comprising a lipid nanoparticle and a pharmaceutically acceptable excipient; wherein the lipid nanoparticle comprises a lipid and the nucleic acid of claim 1.

64. A pharmaceutical composition comprising the nucleic acid of claim 1 and a pharmaceutically acceptable excipient.

65-72. (canceled)

73. A method of treating severe acute respiratory syndrome in a subject in need thereof, the method comprising administering to the subject an effective amount of the nucleic acid of claim 1.

74. A method of treating severe acute respiratory syndrome in a subject in need thereof, the method comprising administering to the subject an effective amount of a lipid nanoparticle; wherein the lipid nanoparticle comprises a lipid and the nucleic acid of claim 1.

75. A method of treating severe acute respiratory syndrome in a subject in need thereof, the method comprising administering to the subject an effective amount of a pharmaceutical composition comprising a lipid nanoparticle and a pharmaceutically acceptable excipient; wherein the lipid nanoparticle comprises a lipid and the nucleic acid of claim 1.

76. The method of claim 73, wherein the severe acute respiratory syndrome is SARS-associated coronavirus (SARS-CoV), SARS-associated coronavirus 1 (SARS-CoV-1) SARS-associated coronavirus 2 (SARS-CoV-2), MERS-associated coronavirus (MERS-CoV).

77-83. (canceled)

84. A drug delivery device comprising an effective amount of the nucleic acid of claim 1.

85. A drug delivery device comprising an effective amount of a lipid nanoparticle comprising a lipid and the nucleic acid of claim 1.

86. A drug delivery device comprising an effective amount of a pharmaceutical composition comprising a lipid nanoparticle and a pharmaceutically acceptable excipient; wherein the lipid nanoparticle comprises a lipid and the nucleic acid of claim 1.

87-88. (canceled)
